(19) United States
(12) Patent Application Publication
(10) Pub. No.: US 2026/0274906 A1
(43) Pub. Date: Sep. 17, 2026

(54) ALLERGY ANTIGEN AND EPITOPE FOR SAME

(71) Applicant: HOYU CO., LTD., Aichi (JP)

(72) Inventors: Kayoko Matsunaga, Aichi (JP); Akiko Yagami, Aichi (JP); Naoshi Shimojo, Aichi (JP); Fumiaki Ohno, Aichi (JP)

(73) Assignee: HOYU CO., LTD., Aichi (JP)

(21) Appl. No.: 19/656,216

(22) Filed: Apr. 23, 2026

Related U.S. Application Data
(62) Division of application No. 18/147,789, filed on Dec. 29, 2022, now Pat. No. 12,630,594.
Application No. 18/147,789 is a division of application No. 16/608,361, filed on Dec. 20, 2017, now abandoned.

(30) Foreign Application Priority Data
Apr. 28, 2017 (WO) PCT/JP2017/017018

(51) Int. Cl. C07K 14/46; A23L 17/00; A23L 17/30; A23L 33/00; A61K 39/00; A61K 39/35; A61P 37/08; C07K 14/47; C12N 15/09; C12Q 1/48; C12Q 1/6888; G01N 33/53; G01N 33/68

(52) U.S. Cl. CPC C07K 14/461; A23L 17/00; A23L 17/30; A23L 33/40; A61K 39/00; A61K 39/35; A61P 37/08; C12Q 1/48; C12Q 1/6888; G01N 33/53; G01N 33/6854; G01N 33/6893; A23V 2002/00; C07K 14/47; C12N 15/09; G01N 2800/24

(57) ABSTRACT

The present invention provides novel antigens of an allergy to fish, methods and kits for diagnosing an allergy to fish, pharmaceutical compositions comprising such an antigen, fishes, fish eggs or processed products of such fish or fish egg in which such an antigen is eliminated, fishes that deliver such fish eggs or are born from such fish egg, and a tester for determining the presence or absence of a fish antigen in an object of interest. The present invention also relates to polypeptides comprising an epitope of an antigen, kits, compositions and methods for diagnosing an allergy, comprising such a polypeptide, pharmaceutical compositions comprising such a polypeptide, and raw materials or processed products in which an antigen comprising such a polypeptide is eliminated or reduced. The present invention further relates to a tester for determining the presence or absence of an antigen in an object of interest.

### CROSS-REFERENCE TO RELATED APPLICATIONS

This application is a divisional application of U.S. application Ser. No. 18/147,789, filed on Dec. 29, 2022, which is a divisional application of U.S. application Ser. No. 16/608,361, filed on Oct. 25, 2019, now abandoned, which is a U.S. National Stage Filing under 35 U.S.C. § 371(c), of International Application No. PCT/JP2017/045832, filed on Dec. 20, 2017, which claims foreign priority to International Patent Application No. PCT/JP2017/017018, filed on Apr. 28, 2017. The entire contents of each of the aforementioned applications are incorporated herein by reference.

### REFERENCE TO SEQUENCE LISTING

The instant application contains a Sequence Listing XML filed which has been submitted electronically in XML format and is hereby incorporated by reference in its entirety. The Sequence Listing XML file, created on May 29, 2026, is named 129249_00804_SL.xml and is 651,015 bytes in size. The Sequence Listing XML file is part of the specification, and is incorporated in its entirety by reference herein.

### TECHNICAL FIELD

The present invention relates to a novel antigen of an allergy to fish. The present invention also relates to a kit, a composition, and a method for diagnosing allergy to fish. The present invention also relates to a pharmaceutical composition comprising such an antigen, fish, fish egg or processed products of such fish or fish egg in which such an antigen is eliminated or reduced, and fish that delivers such fish egg or is born from such fish egg. The present invention further relates to a tester for determining the presence or absence of a fish antigen in an object of interest.

The present invention also relates to a polypeptide comprising an epitope of an antigen. The present invention also relates to a kit, a composition and a method for diagnosing an allergy, comprising such a polypeptide. The present invention also relates to a pharmaceutical composition comprising such a polypeptide, and a raw material or a processed product in which such a polypeptide is eliminated or reduced. The present invention further relates to a method for producing a processed product in which such a polypeptide is eliminated or reduced. The present invention further relates to a tester for determining the presence or absence of an antigen comprising such a polypeptide in an object of interest.

### BACKGROUND ART

In blood and tissues of allergic patients, IgE antibodies specific to particular antigens are produced. Physiological consequences caused by interaction between such IgE antibodies and such particular antigens elicit allergic reactions.

In the process of production of conventional allergy testing agents, antigen reagents are commonly prepared simply by grinding a candidate allergenic food, cooking ingredients or the like (Patent Literature 1). Hence, the reagents contain an enormous number of proteins and contain these individual proteins in very small amounts. For this reason, the only case where conventional allergy tests have permitted detection of a positive allergic reaction is when in a conventional antigen reagent containing many types of proteins, a protein acting as a particular antigen protein (an allergen component) that causes such allergic reaction is present in an amount exceeding a threshold that allows determination of a positive reaction for binding to an IgE antibody. However, no determination of a positive reaction was possible and diagnosis efficiency was not sufficiently high when using a conventional allergy testing agent in patients possessing an IgE antibody binding to an allergen component present in small amounts in an allergen such as food and cooking ingredients.

The severity and symptoms of an allergic reaction do not necessarily correlate with the content of an allergen component. Even when a patient's IgE antibody reacts with an allergen component present in trace amounts in a candidate allergic food, cooking ingredients or the like, the allergic reaction may develop allergic symptoms or may affect the severity of those symptoms.

An attempt to increase the diagnostic efficiency is being made by examining IgE antibodies to protein composing food and cooking ingredients to distinguish sensitization that directly contributes to a diagnosis from sensitization based on cross-antigenicity by a pan-allergen or the like. Fish allergens shown in the table below, etc. are currently known (Non Patent Literature 1-4).

TABLE 1

| | | | MW | | Food | | Modified |
|---|---|---|---|---|---|---|---|
| Species | Allergen | Biochemical name | (SDS-PAGE) | | Allergen | Entry Date | Date |
| Clupea harengus | Clu h 1 | Beta-parvalbumin | 12 | kDa | Yes | 2016 Apr. 4 | 2014 Nov. 3 |
| (Atlantic herring) | | | | | | | |
| Cyprinus carpio | Cyp c 1 | beta-parvalbumin | 12 | | Yes | 2016 Apr. 4 | 2013 Sep. 10 |
| (Common carp) | | | | | | | |
| Gadus callarias | Gad c 1 | Beta-parvalbumin | 12 | | Yes | 2016 Apr. 4 | 2010 Apr. 29 |
| (Baltic cod) | | | | | | | |
| Gadus morhua | Gad m 1 | Beta-parvalbumin | 12 | | Yes | 2016 Apr. 4 | 2011 Jan. 25 |
| (Atlantic cod) | Gad m 2 | Beta-enolase | 47.3 | kDa | Yes | 2016 Apr. 4 | 2012 Jul. 30 |
| | Gad m 3 | Aldolase A | 40 | kDa | Yes | 2016 Apr. 4 | 2012 Jul. 30 |
| Lates calcarifer | Lat c 1 | Beta-parvalbumin | 11.5 | kDa | Yes | 2016 Apr. 4 | 2014 Nov. 13 |
| (Barramundi) | | | | | | | |
| Lepidorhombus whiffiagonis | Lep w 1 | Beta-parvalbumin | 11.5 | kDa | Yes | 2016 Apr. 4 | 2010 Apr. 29 |
| (Megrim, Whiff, Gallo) | | | | | | | |
| Oncorhynchus keta | Onc k 5 | beta-prime-component | 18 | kDa | Yes | 2016 Apr. 4 | 2012 Dec. 18 |
| (Chum salmon) | | of vitellogenin | | | | | |
| Oncorhynchus mykiss | Onc m 1 | Beta-parvalbumin | 12 | kDa | Yes | 2016 Apr. 4 | 2011 Apr. 14 |
| (Rainbow trout) | | | | | | | |
| Oreochromis mossambicus | Ore m 4 | Tropomyosin | 33 | kDa | Yes | 2016 Apr. 4 | 2012 Jul. 30 |
| (Mozambique tilapia) | | | | | | | |
| Salmo salar | Sal s 1 | Beta-parvalbumin 1 | 12 | | Yes | 2016 Apr. 4 | 2010 Apr. 29 |
| (Atlantic salmon) | Sal s 2 | Beta-Enolase | 47.3 | kDa | Yes | 2016 Apr. 4 | 2011 Aug. 26 |
| | Sal s 3 | Aldolase A | 40 | | Yes | 2016 Apr. 4 | 2012 Jul. 30 |
| Sardinops sagax | Sar sa 1 | Beta-parvalbumin | 12 | kDa | Yes | 2016 Apr. 4 | 2010 Apr. 29 |
| (Pacific pilchard) | | | | | | | |
| Sebastes marinus | Seb m 1 | Beta-parvalbumin | 11 | kDa | Yes | 2016 Apr. 4 | 2010 Apr. 29 |
| (Ocean perch, redfish, snapper) | | | | | | | |
| Thunnus albacares | Thu a 1 | Beta-parvalbumin | 11 | kDa | Yes | 2016 Apr. 4 | 2010 Apr. 29 |
| (Yellowfin tuna) | Thu a 2 | Beta-enolase | 50 | | Yes | 2016 Apr. 4 | 2012 Jul. 30 |
| | Thu a 3 | Aldolase A | 40 | | Yes | 2016 Apr. 4 | 2012 Jul. 30 |
| Xiphias gladius | Xip g 1 | Beta-parvalbumin | 11.5 | kDa | Yes | 2016 Apr. 4 | 2010 Apr. 29 |
| (Swordfish) | | | | | | | |

However, while it is necessary to exhaustively identify allergen components in candidate allergic foods and cooking ingredients in order to enhance the reliability of allergy tests, the patient detection rate by the measurement of such allergenic components is far insufficient. Identification of novel allergens in fish is very important not only for increasing the precision of diagnosis, but also for determining targets of therapeutic agents, low allergenic foods, and low allergenic cooking ingredients.

Meanwhile, in the field of protein separation and purification, various efforts have conventionally been made to develop methods for separating and purifying a protein or nucleic acid of interest from cell extracts or the like. Such methods may well be exemplified by dialysis based on salt concentration, and centrifugal separation.

Other efforts have been made to develop many purification methods based on electric charges of protein or nucleic acid residues or on the difference in molecular weight. Electric charge-based purification methods can be exemplified by column chromatography using ion exchange resins, and isoelectric focusing. Purifications based on molecular weight difference can be exemplified by centrifugal separation, molecular-sieve column chromatography, and SDS-PAGE (sodium dodecyl sulfate-polyacrylamide gel electrophoresis).

In recent years, a method for separating and purifying many different proteins from a small amount of sample has been used, which is more specifically a two-dimensional electrophoresis consisting of isoelectric focusing in the first dimension, followed by SDS-PAGE in the second dimension. The present applicant has conventionally developed some 2D electrophoresis methods with high separation ability (Patent Literature 2-5).

Allergen-specific IgE antibodies recognize and bind to epitopes that are particular amino acid sequences in allergen components. However, only a slight number of analyses have been made on epitopes as to the allergen components (Non Patent Literature 5), but such analyses are still totally quite rare. Furthermore, any kit for diagnosing an allergy using a polypeptide comprising an epitope has not yet emerged in the market.

### CITATION LIST

### Patent Literature

PTL1: Japanese Patent Application Publication No. JP 2002-286716
PTL2: Japanese Patent Application Publication No. JP 2011-33544
PTL3: Japanese Patent Application Publication No. JP 2011-33546
PTL4: Japanese Patent Application Publication No. JP 2011-33547
PTL5: Japanese Patent Application Publication No. JP 2011-33548

### Non Patent Literature

NPL 1: Allergen Nomenclature, WHO/IUIS Allergen Nomenclature Sub-Committee, [searched on Jun. 9, 2016], Internet <URL: http://www.allergen.org/search.php?allergenname=&allergensource=&TaxSource=Animalia+Chordata&TaxOrder=&foodallerg=1&bioname=>
NPL 2: Kuehn, A., et al., Clin. Exp. Allergy, (2013), Vol. 43, No. 7, pp. 811-22
NPL 3: Gonzalez-Mancebo, E., et al., Ann. Allergy Asthma Immunol., (2014), Vol. 113, No. 1, pp. 114-115
NPL 4: van der Ventel, M. L., et al., Mol. Immunol., (2011), Vol. 48, No. 4, pp. 637-646
NPL 5: Matsuo, H., et al., J. Biol. Chem., (2004), Vol. 279, No. 13, pp. 12135-12140

### SUMMARY OF INVENTION

### Technical Problem

The present invention provides novel antigens of an allergy to fish. The present invention also provides methods and kits for diagnosing allergy to fish. The present invention also provides pharmaceutical compositions comprising such an antigen, fish, fish egg or processed products of such fish or fish egg in which such an antigen is eliminated or reduced, and fish that delivers such fish egg or is born from such fish egg. The present invention further provides testers for determining the presence or absence of a fish antigen in an object of interest.

The present invention also provides polypeptides comprising an epitope of an antigen. The present invention also provides kits, compositions and methods for diagnosing an allergy including such a polypeptides. The present invention also provides pharmaceutical compositions comprising such a polypeptide, and raw materials or processed products in which an antigen comprising such a polypeptide is eliminated or reduced The present invention further relates to methods for producing a processed product in which such an antigen is eliminated or reduced. The present invention further relates to testers for determining the presence or absence of an antigen comprising such a polypeptide in an object of interest.

### Solution to Problem

In order to solve the aforementioned problems, the present inventors had made intensive studies to identify causative antigens of an allergy to fish. As a result, the inventors succeeded in identifying novel antigens to which an IgE antibody in the serum of a fish allergic patient specifically binds. The present invention has been completed based on this finding.

Thus, in one embodiment, the present invention can be as defined below.
[1] A kit for diagnosing an allergy to a fish, the kit comprising, as an antigen, at least one of proteins as defined below in any one of (10) to (14):
   (10) (10A) myosin heavy chain, fast skeletal muscle-like or a variant thereof, which is an antigen of an allergy to a fish and is defined below in any of (10A-a) to (10A-e):
(10A-a) a protein comprising an amino acid sequence with deletion, substitution, insertion or addition of one or several amino acids in SEQ ID NO: 70;
(10A-b) a protein comprising an amino acid sequence having at least 70% identity to the amino acid sequence of SEQ ID NO: 70;
(10A-c) a protein comprising an amino acid sequence encoded by a nucleotide sequence with deletion, substitution, insertion or addition of one or several nucleotides in SEQ ID NO: 69;
(10A-d) a protein comprising an amino acid sequence encoded by a nucleotide sequence having at least 70% identity to the nucleotide sequence of SEQ ID NO: 69; or
(10A-e) a protein comprising an amino acid sequence encoded by a nucleic acid that hybridizes under stringent conditions with a nucleic acid having a nucleotide sequence complementary to the nucleotide sequence of SEQ ID NO: 69; or
(10B) a protein comprising at least one amino acid sequence selected from the group consisting of SEQ ID NOs: 70-108;
   (11) (11A) glycogen phosphorylase, muscle form-like or a variant thereof, which is an antigen of an allergy to a fish and is defined below in any of (11A-a) to (11A-e):
(11A-a) a protein comprising an amino acid sequence with deletion, substitution, insertion or addition of one or several amino acids in SEQ ID NO: 110;
(11A-b) a protein comprising an amino acid sequence having at least 70% identity to the amino acid sequence of SEQ ID NO: 110;
(11A-c) a protein comprising an amino acid sequence encoded by a nucleotide sequence with deletion, substitution, insertion or addition of one or several nucleotides in SEQ ID NO: 109;
(11A-d) a protein comprising an amino acid sequence encoded by a nucleotide sequence having at least 70% identity to the nucleotide sequence of SEQ ID NO: 109; or
(11A-e) a protein comprising an amino acid sequence encoded by a nucleic acid that hybridizes under stringent conditions with a nucleic acid having a nucleotide sequence complementary to the nucleotide sequence of SEQ ID NO: 109; or
(11B) a protein comprising at least one amino acid sequence selected from the group consisting of SEQ ID NOs: 110-119;
   (12) (12A) myosin-binding protein C, fast-type-like or a variant thereof, which is an antigen of an allergy to a fish and is defined below in any of (12A-a) to (12A-e):
(12A-a) a protein comprising an amino acid sequence with deletion, substitution, insertion or addition of one or several amino acids in SEQ ID NO: 121;
(12A-b) a protein comprising an amino acid sequence having at least 70% identity to the amino acid sequence of SEQ ID NO: 121;
(12A-c) a protein comprising an amino acid sequence encoded by a nucleotide sequence with deletion, substitution, insertion or addition of one or several nucleotides in SEQ ID NO: 120;
(12A-d) a protein comprising an amino acid sequence encoded by a nucleotide sequence having at least 70% identity to the nucleotide sequence of SEQ ID NO: 120; or
(12A-e) a protein comprising an amino acid sequence encoded by a nucleic acid that hybridizes under stringent conditions with a nucleic acid having a nucleotide sequence complementary to the nucleotide sequence of SEQ ID NO: 120; or
(12B) a protein comprising at least one amino acid sequence selected from the group consisting of SEQ ID NOs: 121-136;
   (13) (13A) ATP synthase subunit beta, mitochondrial or a variant thereof, which is an antigen of an allergy to a fish and is defined below in any of (13A-a) to (13A-e):
(13A-a) a protein comprising an amino acid sequence with deletion, substitution, insertion or addition of one or several amino acids in SEQ ID NO: 138;
(13A-b) a protein comprising an amino acid sequence having at least 70% identity to the amino acid sequence of SEQ ID NO: 138;
(13A-c) a protein comprising an amino acid sequence encoded by a nucleotide sequence with deletion, substitution, insertion or addition of one or several nucleotides in SEQ ID NO: 137;
(13A-d) a protein comprising an amino acid sequence encoded by a nucleotide sequence having at least 70% identity to the nucleotide sequence of SEQ ID NO: 137; or
(13A-e) a protein comprising an amino acid sequence encoded by a nucleic acid that hybridizes under stringent conditions with a nucleic acid having a nucleotide sequence complementary to the nucleotide sequence of SEQ ID NO: 137; or
(13B) a protein comprising at least one amino acid sequence selected from the group consisting of SEQ ID NOs: 138-142; and
   (14) (14A) L-lactate dehydrogenase A chain-like or a variant thereof, which is an antigen of an allergy to a fish and is defined below in any of (14A-a) to (14A-e):
(14A-a) a protein comprising an amino acid sequence with deletion, substitution, insertion or addition of one or several amino acids in SEQ ID NO: 144;
(14A-b) a protein comprising an amino acid sequence having at least 70% identity to the amino acid sequence of SEQ ID NO: 144;
(14A-c) a protein comprising an amino acid sequence encoded by a nucleotide sequence with deletion, substitution, insertion or addition of one or several nucleotides in SEQ ID NO: 143;
(14A-d) a protein comprising an amino acid sequence encoded by a nucleotide sequence having at least 70% identity to the nucleotide sequence of SEQ ID NO: 143; or
(14A-e) a protein comprising an amino acid sequence encoded by a nucleic acid that hybridizes under stringent conditions with a nucleic acid having a nucleotide sequence complementary to the nucleotide sequence of SEQ ID NO: 143; or
(14B) a protein comprising at least one amino acid sequence selected from the group consisting of SEQ ID NOs: 144-149;
[2] A composition for diagnosing an allergy to fish, comprising, as an antigen, at least one of proteins as defined above in any of (10) to (14) of [1].
[3] A method for providing an indicator for diagnosing an allergy to fish in a subject, the method comprising the steps of:
   (i) contacting a sample obtained from the subject with an antigen, wherein the sample is a solution comprising an IgE antibody;
   (ii) detecting binding between the IgE antibody present in the sample from the subject and the antigen; and
   (iii) when the binding between the IgE antibody in the subject and the antigen is detected, an indicator of the fact that the subject is allergic to fish is provided; wherein the antigen is at least one of proteins as defined above in any of (10) to (14) of [1].
[4] A pharmaceutical composition comprising at least one of proteins as defined above in any of (10) to (14) of [1].
[5] The pharmaceutical composition as set forth in [4], wherein the pharmaceutical composition is intended for the treatment of an allergy to fish.
[6] Fish or processed products of fish in which an antigen is eliminated or reduced, wherein the antigen is at least one of proteins as defined above in any of (10) to (14) of [1].
[7] A tester for determining the presence or absence of a fish antigen in an object of interest, comprising an antibody that binds to at least one of proteins as defined above in any of (10) to (14) of [1].
[8] A tester for determining the presence or absence of an antigen causative of an allergy to fish in an object of interest, comprising a primer having a nucleotide sequence complementary to a portion of at least one of the nucleotide sequences of SEQ ID NOs: 69, 109, 120, 137 and 143.
[9] A fish-derived antigen which is at least one of proteins as defined above in any of (10) to (14) of [1] and is causative of an allergy to fish.

The present inventors also succeeded in finding epitopes as to fish-derived antigens including the antigens described above.

Since the epitopes have a relatively short amino acid sequence, the IgE antibodies are capable of binding to different allergen components if the same amino acid sequence is present in the different allergen components. Since different allergen components have a common epitope so that IgE antibodies from allergic patients bind to both of them, the antigens have cross-reactivity. Thus, the epitopes defined in the present invention enable diagnosis or treatment of an allergy including cross-reactivity, and detection of a plurality of allergen components comprising the epitopes, etc.

The present invention has been completed based on this finding. Thus, in another embodiment, the present invention can be as defined below.
[10] A polypeptide specifically binding to an IgE antibody from an allergic patient, the polypeptide being any one of the following:
   (1α) a polypeptide comprising at least one amino acid sequence selected from the group consisting of SEQ ID NOs: 150-154 and 205-227;
   (2α) a polypeptide comprising at least one amino acid sequence selected from the group consisting of SEQ ID NOs: 155 and 228-230;
   (3α) a polypeptide comprising at least one amino acid sequence selected from the group consisting of SEQ ID NOs: 156, 157 and 231-237;
   (4α) a polypeptide comprising at least one amino acid sequence selected from the group consisting of SEQ ID NOs: 158, 159 and 238-247;
   (5α) a polypeptide comprising at least one amino acid sequence selected from the group consisting of SEQ ID NOs: 160-162 and 248-261;
   (6α) a polypeptide comprising at least one amino acid sequence selected from the group consisting of SEQ ID NOs: 163-167 and 262-279;
   (7α) a polypeptide comprising at least one amino acid sequence selected from the group consisting of SEQ ID NOs: 168-171 and 280-300;
   (8α) a polypeptide comprising at least one amino acid sequence selected from the group consisting of SEQ ID NOs: 172-174 and 301-310;
   (9α) a polypeptide comprising at least one amino acid sequence selected from the group consisting of SEQ ID NOs: 175-178 and 311-326;
   (10α) a polypeptide comprising at least one amino acid sequence selected from the group consisting of SEQ ID NOs: 179-185 and 327-365;
   (11α) a polypeptide comprising at least one amino acid sequence selected from the group consisting of SEQ ID NOs: 186 and 366-370;
   (12α) a polypeptide comprising at least one amino acid sequence selected from the group consisting of SEQ ID NOs: 187-196 and 371-413;
   (13α) a polypeptide comprising at least one amino acid sequence selected from the group consisting of SEQ ID NOs: 197 and 414-417;
   (14α) a polypeptide comprising at least one amino acid sequence selected from the group consisting of SEQ ID NOs: 198 and 418-420;
   (15α) a polypeptide comprising at least one amino acid sequence selected from the group consisting of SEQ ID NOs: 199 and 421-425;
   (16α) a polypeptide comprising at least one amino acid sequence selected from the group consisting of SEQ ID NOs: 200-202 and 426-436; and
   (17α) a polypeptide comprising at least one amino acid sequence selected from the group consisting of SEQ ID NOs: 203, 204 and 437-444.
[11] The polypeptide according to the above [10], wherein the number of amino acid residues is 500 or less.
[12] A kit for diagnosing an allergy, comprising at least one of polypeptides according to the above [10] or [11].
[13] A composition for diagnosing an allergy, the composition comprising at least one of polypeptides according to the above [10] or [11] as an antigen.
[14] A method for providing an indicator for diagnosing an allergy in a subject, the method comprising the steps of:
(i) contacting a sample obtained from the subject with an antigen, wherein the sample is a solution comprising an IgE antibody;
(ii) detecting binding between the IgE antibody present in the sample obtained from the subject and the antigen; and
(iii) when the binding between the IgE antibody in the subject and the antigen is detected, an indicator of the fact that the subject is allergic is provided;
wherein the antigen is at least one of polypeptides according to any one of the above [10] or [11].
[15] A pharmaceutical composition comprising at least one of polypeptides according to the above [10] or [11].
[16] The pharmaceutical composition according to the above [15], wherein the pharmaceutical composition is intended for the treatment of an allergy.
[17] A tester for determining the presence or absence of an antigen in an object of interest, the tester comprising an antibody that binds to at least one of polypeptides according to the above [10] or [11].
[18] A tester for determining the presence or absence of an antigen in an object of interest, the tester comprising any of the following primers:
   (a) a primer comprising a portion of the nucleotide sequence of a nucleic acid encoding a polypeptide according to the above [10] or [11], and/or a portion of a complementary strand thereof; and
   (b) a primer which is a portion of at least one of the nucleotide sequences of SEQ ID NOs: 69, 109, 120, 137 and 143 and/or a primer which is a portion of a sequence complementary to at least one of the nucleotide sequences of SEQ ID NOs: 69, 109, 120, 137 and 143.
[19] A raw material or a processed product in which an antigen is eliminated or reduced, wherein the antigen is at least one of polypeptides according to the above [10] or [11].
[20] A method for producing a processed product in which an antigen is eliminated or reduced, the method comprising the step of confirming that the antigen is eliminated or reduced in a production process of the processed product, wherein the antigen is at least one of polypeptides according to the above [10] or [11].

### Advantageous Effects of Invention

The present invention can provide novel antigens of an allergy to fish. Since the novel antigens (allergen components) that trigger a fish allergy were identified according to this invention, this invention can provide highly sensitive methods and kits for diagnosing an allergy to fish, pharmaceutical compositions comprising such an antigen, fish, fish egg, or processed products of such fish or fish egg in which such an antigen is eliminated or reduced, fish that delivers such fish egg or is born from such fish egg, and testers for determining the presence or absence of a fish antigen in an object of interest.

The present invention can provide novel polypeptides comprising an epitope of an antigen. Use of the polypeptide of the present invention enables provision of highly sensitive kits, compositions and methods for diagnosing an allergy, pharmaceutical compositions comprising such a polypeptide, testers for determining the presence or absence of an antigen comprising such a polypeptide in an object of interest, raw materials or processed products in which such a polypeptide is eliminated or reduced, and a method for producing the processed products.

### BRIEF DESCRIPTION OF DRAWINGS

FIG. 1A is a photograph of a gel showing a protein electrophoretic pattern in two-dimensional electrophoresis of proteins contained in an extract of salmon meat (left diagram) and a photograph of an immunoblot of the two-dimensional electrophoretic pattern using the serum of fish-allergic patient 1 (right diagram). The bands at the left of the photograph of the gel are bands of molecular weight markers, and the numeric values at the left of the photograph of the gel are respective molecular weights (KDa) of the molecular weight markers. The numeric values at the top of the photograph represent isoelectric points.

FIG. 1B is a photograph of an immunoblot of a two-dimensional electrophoretic pattern of proteins contained in an extract of salmon meat using the serum of fish-allergic patient 2 (left diagram) and using the serum of fish-allergic patient 3 (right diagram). The numeric values at the top of each photograph represent isoelectric points.

FIG. 2 is a photograph of an immunoblot of a two-dimensional electrophoretic pattern of proteins contained in meat extracts of 10 types of fishes using the serum of fish-allergic patient 1.

FIG. 3 is a photograph of an immunoblot of a two-dimensional electrophoretic pattern of proteins contained in meat extracts of 10 types of fishes using the serum of a subject having no symptom of a fish allergy (non-fish-allergic subject).

FIG. 4 is a photograph of an immunoblot of a two-dimensional electrophoretic pattern of proteins contained in meat extracts of 6 types of fishes using the serum of fish-allergic patient 4.

FIG. 5 is a diagram showing results of measuring chemiluminescence using the sera of a fish-allergic patient and a non-fish-allergic subject as to a peptide of epitope No. 11 (SEQ ID NO: 160) having the amino acid sequence “SMVLVKMKEIAEAYL” derived from salmon heat shock cognate 70 kDa protein, and a peptide having the amino acid sequence “AMVLVKMKETAEAYL” derived from bastard halibut heat shock cognate 70 kDa protein. The numeric values on the ordinate represent absorbance for light at a wavelength of 450 nm.

### DESCRIPTION OF EMBODIMENTS

The present invention will be described in detail below, but the present invention is not limited to them.

Unless otherwise defined herein, all scientific and technical terms used in relation to the present invention shall have meanings commonly understood by those skilled in the art.

As referred to herein, the “allergy” refers to the state in which, when a certain antigen enters the body of a living individual sensitized to said antigen, the living individual shows a hypersensitive reaction detrimental to him/her. An allergic reaction can be produced upon contact with an antigen or consumption of the antigen. Here, the contact refers to touch to an object and, particularly, as for the human body, refers to attachment to the skin, the mucosa (eyes, lips, etc.) or the like. The consumption refers to incorporation into the body and refers to incorporation by inhalation, through an oral route, or the like. In general, allergic reactions caused by consumption of foods are particularly referred to as food allergies. In a preferred mode, the allergy may be a food allergy. In blood and tissues of individuals with many food-allergic diseases, IgE antibodies specific to antigens are produced. IgE antibodies bind to mast cells or basophils. When an antigen specific to such an IgE antibody enters again the body of a patient with an allergic disease, said antigen combines with the IgE antibody bound to mast cells or basophils, and the IgE antibody crosslinks said antigen on the cell surface, resulting in physiological effects of IgE antibody-antigen interaction. Examples of such physiological effects include release of histamine, serotonin, heparin, eosinophil chemotactic factors, leucotrienes, or the like. These released substances provoke an allergic reaction resulting from the combination of an IgE antibody with particular antigens. Specifically, IgE antibodies recognize and bind to epitopes that are particular amino acid sequences in particular antigens. Allergic reactions caused by such antigens occur through the aforementioned pathway.

In the present invention, the allergy of interest is not particularly limited as long as it is an allergy to an allergen comprising an epitope to be used. Such an allergy may include allergies to plants of the family Oleaceae, the family Compositae, the family Poaceae, the family Bromeliaceae, the family Juglandaceae, the family Cucurbitaceae, and the family Leguminosae, allergies to animals of the family Phasianidae and the family Bovidae, allergies to seafood of the family Carangidae, the family Sparidae, the family Salmonidae, the family Penaeidae, the family Lithodidae, the family Octopodidae, and the family Veneridae, and parasites of the family Anisakidae. Examples of the plant of the family Oleaceae include olive (scientific name: Olea europaea). Examples of the plant of the family Compositae include artichoke (scientific name: Cynara scolymus). Examples of the plant of the family Poaceae include bread wheat (scientific name: Triticum aestivum) and Tausch's goatgrass (scientific name: Aegilops tauschii). Examples of the plant of the family Bromeliaceae include pineapple (scientific name: Ananas comosus). Examples of the plant of the family Juglandaceae include walnut (scientific name: Juglans regia). Examples of the plant of the family Cucurbitaceae include squash (scientific name: Cucurbita moschata). Examples of the plant of the family Leguminosae include soybean (scientific name: Glycine max). Examples of the animal of the family Phasianidae include Japanese quail (scientific name: Coturnix japonica) and wild turkey (scientific name: Meleagris gallopavo). Examples of the animal of the family Bovidae include cattle (scientific name: Bos taurus). Examples of the seafood of the family Carangidae include greater amberjack (scientific name: Seriola dumerili). Examples of the seafood of the family Sparidae include red seabream (scientific name: Pagrus major). Examples of the seafood of the family Salmonidae include rainbow trout (scientific name: Oncorhynchus mykiss). Examples of the seafood of the family Penaeidae include whiteleg shrimp (scientific name: Litopenaeus vannamei). Examples of the seafood of the family Lithodidae include red king crab (scientific name: Paralithodes camtschaticus). Examples of the seafood of the family Octopodidae include North Pacific giant octopus (scientific name: Enteroctopus dofleini). Examples of the seafood of the family Veneridae include Manila clam (scientific name: Ruditapes philippinarum). Examples of the parasite of the family Anisakidae include Anisakis (Anisakis simplex).

As referred to herein, the fish means, among fishes, a bony fish or a cartilage fish, preferably a bony fish, more preferably a fish belonging to the order Salmoniformes, the order Perciformes, the order Anguilliformes, the order Gadiformes, or the order Pleuronectiformes, further preferably a fish belonging to the family Salmonidae, the family Carangidae, the family Congridae, the family Sparidae, the family Scombridae, the family Gadidae, the family Anguillidae, or the family Paralichthyidae, still further preferably salmon, horse mackerel, conger, blackhead seabream, mackerel, sea bream, cod, amberjack, eel, or bastard halibut. The fish may be edible.

As referred to herein, the “fish egg” means an egg of a fish and is discriminated from the “egg”, which typically means an egg of a bird. The fish egg may be edible.

As referred to herein, the allergy to fish refers to the state in which an individual has an allergic reaction caused by proteins, etc. present in fish which act as an antigen. The allergy to fish can produce an allergic reaction upon contact with an antigen present in fish or consumption of the antigen. In general, allergic reactions caused by consumption of foods are particularly referred to as food allergies. The allergy to fish may be a food allergy.

As referred to herein, the “antigen” refers to a substance that provokes an allergic reaction, and is also referred to as an “allergen component”. The antigen is preferably a protein.

As referred to herein, the “protein” refers to a molecule having a structure in which naturally occurring amino acids are joined together by peptide bond. The number of amino acids present in a protein is not particularly limited. As referred to herein, the term “polypeptide” also means a molecule having a structure in which naturally occurring amino acids are joined together by peptide bond. The number of amino acids present in a polypeptide is not particularly limited. The “polypeptide” conceptually includes the “protein”. Also, polypeptides having about 2 to 50 amino acids joined together by peptide bond are in some cases called “peptides”, especially. In the case where amino acids can form different enantiomers, the amino acids are understood to form an L-enantiomer, unless otherwise indicated. The amino acid sequences of proteins, polypeptides, or peptides as used herein are represented by one-letter symbols of amino acids in accordance with standard usage and the notational convention commonly used in the art. The leftward direction represents the amino-terminal direction, and the rightward direction represents the carboxy-terminal direction. In the one-letter symbols of amino acids, X can be any substance having an amino group and a carboxyl group that can bind to amino acids at both ends, and particularly represents that any of 20 types of naturally occurring amino acids are acceptable. The residue of X is an amino acid residue at a site where binding activity against IgE antibodies from allergic patients was maintained even after substitution by alanine in alanine scanning described in Example 10. It is well known to those skilled in the art that even when such a site is substituted by any other amino acids, it is highly probable that this binding activity against IgE antibodies is maintained.

### Identification of Antigens

Proteins contained in fish were analyzed by the aforementioned technique to identify antigens of an allergy to fish. To be specific, proteins were extracted from fish meat and subjected to two-dimensional electrophoresis under the conditions described below.

The electrophoresis in the first dimension was isoelectric focusing, which was performed using isoelectric focusing gels with a gel-strip length of 5 to 10 cm and a gel pH range of 3 to 10. The pH gradient of the gels in the direction of electrophoresis was as follows: with the total gel-strip length being taken as 1, the gel-strip length up to pH 5 was “a=0.15 to 0.3”, the gel-strip length from pH 5 to 7 was “b=0.4 to 0.7”, and the gel-strip length above pH 7 was “c=0.15 to 0.3”. More specifically, the isoelectric focusing was performed using the IPG gels, Immobiline Drystrip (pH3-10NL), produced by GE Healthcare Bio-Sciences Corporation (hereinafter abbreviated as “GE”). The electrophoresis system used was IPGphor produced by GE. The maximum current of the electrophoresis system was limited to 75 μA per gel strip. The voltage program adopted to perform the first-dimensional isoelectric focusing was as follows: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

The electrophoresis in the second dimension was SDS-PAGE, which was performed using polyacrylamide gels whose gel concentration at the distal end in the direction of electrophoresis was set to 3 to 6% and whose gel concentration at the proximal end was set to a higher value than that at the distal end. More specifically, the SDS-PAGE was performed using NuPAGE 4-12% Bris-Tris Gels (IPG well, Mini, 1 mm) produced by Life Technologies. The electrophoresis system used was XCell SureLock Mini-Cell produced by Life Technologies. The electrophoresis was run at a constant voltage of 200 V for about 45 minutes using an electrophoresis buffer composed of 50 mM MOPS, 50 mM Tris base, 0.1% (w/v) SDS and 1 mM EDTA.

As a result, the following spots in a two-dimensional electrophoresis gel run under the conditions described above for proteins in fish have been revealed to exhibit specific binding to IgE antibodies from fish-allergic patients diagnosed with immediate-type allergy.
Spot 1: Molecular weight 80 to 160 kDa, pI 3.0 to 7.0
Spot 2: Molecular weight 110 to 260 kDa, pI 4.0 to 8.0
Spot 3: Molecular weight 80 to 160 kDa, pI 4.0 to 10.0
Spot 4: Molecular weight 80 to 160 kDa, pI 5.0 to 11.0
Spot 5: Molecular weight 50 to 110 kDa, pI 3.0 to 7.0
Spot 6: Molecular weight 60 to 110 kDa, pI 4.0 to 8.0
Spot 7: Molecular weight 40 to 80 kDa, pI 4.0 to 8.0
Spot 8: Molecular weight 40 to 80 kDa, pI 3.0 to 7.0
Spot 9: Molecular weight 20 to 50 kDa, pI 3.0 to 7.0
Spot 10: Molecular weight 160 to 300, pI 3.0 to 7.0
Spot 11: Molecular weight 80 to 160, pI 4.0 to 8.0
Spot 12: Molecular weight 100 to 160, pI 4.0 to 7.0
Spot 13: Molecular weight 30 to 70, pI 3.0 to 7.0
Spot 14: Molecular weight 20 to 50, pI 5.0 to 9.0 Antigen

### (10) Antigen in Spot 10

As the result of sequence identification of the antigen in spot 10 by mass spectroscopy, the amino acid sequences of SEQ ID NOs: 71-108 were detected.

Also, the mass spectroscopic data (SEQ ID NOs: 71-108) obtained for spot 10 on a mass spectrometer was analyzed by comparing the data against the NCBI protein data, and as a result, myosin heavy chain, fast skeletal muscle-like (amino acid sequence: SEQ ID NO: 70, encoding nucleotide sequence: SEQ ID NO: 69) was identified. SEQ ID NO: 71 corresponds to amino acids 13-19 of SEQ ID NO: 70, SEQ ID NO: 72 corresponds to amino acids 262-271 of SEQ ID NO: 70, SEQ ID NO: 73 corresponds to amino acids 996-1014 of SEQ ID NO: 70, SEQ ID NO: 74 corresponds to amino acids 951-961 of SEQ ID NO: 70, SEQ ID NO: 75 corresponds to amino acids 1293-1303 of SEQ ID NO: 70, SEQ ID NO: 76 corresponds to amino acids 1082-1091 of SEQ ID NO: 70, SEQ ID NO: 77 corresponds to amino acids 1847-1856 of SEQ ID NO: 70, SEQ ID NO: 78 corresponds to amino acids 172-186 of SEQ ID NO: 70, SEQ ID NO: 79 corresponds to amino acids 919-937 of SEQ ID NO: 70, SEQ ID NO: 80 corresponds to amino acids 249-258 of SEQ ID NO: 70, SEQ ID NO: 81 corresponds to amino acids 706-717 of SEQ ID NO: 70, SEQ ID NO: 82 corresponds to amino acids 50-59 of SEQ ID NO: 70, SEQ ID NO: 83 corresponds to amino acids 415-430 of SEQ ID NO: 70, SEQ ID NO: 84 corresponds to amino acids 834-845 of SEQ ID NO: 70, SEQ ID NO: 85 corresponds to amino acids 617-630 of SEQ ID NO: 70, SEQ ID NO: 86 corresponds to amino acids 1556-1567 of SEQ ID NO: 70, SEQ ID NO: 87 corresponds to amino acids 1391-1408 of SEQ ID NO: 70, SEQ ID NO: 88 corresponds to amino acids 1025-1040 of SEQ ID NO: 70, SEQ ID NO: 89 corresponds to amino acids 1813-1836 of SEQ ID NO: 70, SEQ ID NO: 90 corresponds to amino acids 743-755 of SEQ ID NO: 70, SEQ ID NO: 91 corresponds to amino acids 1172-1192 of SEQ ID NO: 70, SEQ ID NO: 92 corresponds to amino acids 353-364 of SEQ ID NO: 70, SEQ ID NO: 93 corresponds to amino acids 1261-1292 of SEQ ID NO: 70, SEQ ID NO: 94 corresponds to amino acids 1783-1789 of SEQ ID NO: 70, SEQ ID NO: 95 corresponds to amino acids 1502-1519 of SEQ ID NO: 70, SEQ ID NO: 96 corresponds to amino acids 1484-1497 of SEQ ID NO: 70, SEQ ID NO: 97 corresponds to amino acids 1194-1212 of SEQ ID NO: 70, SEQ ID NO: 98 corresponds to amino acids 1304-1314 of SEQ ID NO: 70, SEQ ID NO: 99 corresponds to amino acids 369-384 of SEQ ID NO: 70, SEQ ID NO: 100 corresponds to amino acids 1315-1322 of SEQ ID NO: 70, SEQ ID NO: 101 corresponds to amino acids 1536-1555 of SEQ ID NO: 70, SEQ ID NO: 102 corresponds to amino acids 237-248 of SEQ ID NO: 70, SEQ ID NO: 103 corresponds to amino acids 1699-1725 of SEQ ID NO: 70, SEQ ID NO: 104 corresponds to amino acids 1092-1104 of SEQ ID NO: 70, SEQ ID NO: 105 corresponds to amino acids 407-414 of SEQ ID NO: 70, SEQ ID NO: 106 corresponds to amino acids 1897-1917 of SEQ ID NO: 70, SEQ ID NO: 107 corresponds to amino acids 1458-1470 of SEQ ID NO: 70, and SEQ ID NO: 108 corresponds to amino acids 1373-1388 of SEQ ID NO: 70.

Accordingly, the antigen in spot 10 in the present invention can be any of below in (10A-a) to (10A-e), and (10B):
(10A-a) a protein comprising an amino acid sequence with deletion, substitution, insertion or addition of one or several amino acids in SEQ ID NO: 70;
(10A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 70;
(10A-c) a protein comprising an amino acid sequence encoded by a nucleotide sequence with deletion, substitution, insertion or addition of one or several nucleotides in SEQ ID NO: 69;
(10A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 69;
(10A-e) a protein comprising an amino acid sequence encoded by a nucleic acid that hybridizes under stringent conditions with a nucleic acid having a nucleotide sequence complementary to the nucleotide sequence of SEQ ID NO: 69;
(10B) a protein comprising at least one amino acid sequence selected from the group consisting of SEQ ID NOs: 70-108, preferably a protein comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 types or all sequences of the amino acid sequences. As referred to above, the amino acid sequence of any of SEQ ID NOs: 70 to 108 may be an amino acid sequence derived therefrom by deletion, substitution, insertion or addition of one or several amino acids;

The proteins as defined above in (10A-a) to (10A-e) and (10B) include those proteins whose amino acid residues are modified by phosphorylation, sugar chain modification, aminoacylation, ring-opening, deamination or the like.

The proteins as defined above in (10A-a) to (10A-e) and (10B) can be proteins that are found in a protein spot with a molecular weight of around 160 kDa to 300 kDa, preferably around 160 kDa to 260 kDa, more preferably around 200 kDa to 230 kDa and an isoelectric point of 3.0 to 7.0, preferably 4.0 to 6.0, more preferably 4.5 to 5.5 on gels used in the two-dimensional electrophoresis performed under the conditions described above in the subsection titled “Identification of antigens”.

Preferably, a protein that is an antigen in spot 10 is an antigen of an allergy to fish.

Epitopes of the aforementioned antigens (10A-a) to (10A-e) and (10B) reside in amino acids 61-75 (SEQ ID NO: 179), amino acids 471-485 (SEQ ID NO: 180), amino acids 621-635 (SEQ ID NO: 181), amino acids 981-995 (SEQ ID NO: 182), amino acids 1011-1025 (SEQ ID NO: 183), amino acids 1041-1055 (SEQ ID NO: 184), and amino acids 1741-1755 (SEQ ID NO: 185) of SEQ ID NO: 70. When the antigen in spot 10 contains a variation in the amino acid sequence of SEQ ID NO: 70, amino acids corresponding to at least one of these epitopes may retain the sequence in SEQ ID NO: 70.

From the viewpoint that binding activity against IgE antibodies such as sensitivity (a degree to which a patient can be diagnosed as being positive) or specificity (a degree to which a healthy subject is not diagnosed as being positive) remains even if the amino acid sequence is varied, the antigen in spot 10 may be the following variant.

In the epitope having the sequence of SEQ ID NO: 179, sites of amino acids other than X in SEQ ID NO: 327 or 328 corresponding to the positions of the amino acids at positions 1-11 or SEQ ID NO: 330 or 331 corresponding to the positions of the amino acids at positions 1-9 in SEQ ID NO: 179 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 1-11 of amino acids 61-75 are SEQ ID NO: 327 or 328 or the amino acids at positions 1-9 thereof are SEQ ID NO: 330 or 331.

In the epitope having the sequence of SEQ ID NO: 180, sites of amino acids other than X in SEQ ID NO: 333 or 334 corresponding to the positions of the amino acids at positions 7-15 in SEQ ID NO: 180 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 7-15 of amino acids 471-485 are SEQ ID NO: 333 or 334.

In the epitope having the sequence of SEQ ID NO: 181, sites of amino acids other than X in SEQ ID NO: 336 or 337 corresponding to the positions of the amino acids at positions 1-10 in SEQ ID NO: 181 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 1-10 of amino acids 621-635 are SEQ ID NO: 336 or 337.

In the epitope having the sequence of SEQ ID NO: 182, sites of amino acids other than X in SEQ ID NO: 339 or 340 corresponding to the positions of the amino acids at positions 2-9, SEQ ID NO: 342 or 343 corresponding to the positions of the amino acids at positions 2-11, SEQ ID NO: 345 or 346 corresponding to the positions of the amino acids at positions 7-15, or SEQ ID NO: 348 or 349 corresponding to the positions of the amino acids at positions 9-14 in SEQ ID NO: 182 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 2-9 of amino acids 981-995 are SEQ ID NO: 339 or 340, the amino acids at positions 2-11 thereof are SEQ ID NO: 342 or 343, the amino acids at positions 7-15 thereof are SEQ ID NO: 345 or 346, or the amino acids at positions 9-14 thereof are SEQ ID NO: 348 or 349.

In the epitope having the sequence of SEQ ID NO: 183, sites of amino acids other than X in SEQ ID NO: 351 or 352 corresponding to the positions of the amino acids at positions 5-14 in SEQ ID NO: 183 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 5-14 of amino acids 1011-1025 are SEQ ID NO: 351 or 352.

In the epitope having the sequence of SEQ ID NO: 184, sites of amino acids other than X in SEQ ID NO: 354 or 355 corresponding to the positions of the amino acids at positions 5-12 or SEQ ID NO: 357 or 358 corresponding to the positions of the amino acids at positions 9-15 in SEQ ID NO: 184 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 5-12 of amino acids 1041-1055 are SEQ ID NO: 354 or 355 or the amino acids at positions 9-15 thereof are SEQ ID NO: 357 or 358.

In the epitope having the sequence of SEQ ID NO: 185, sites of amino acids other than X in SEQ ID NO: 360 or 361 corresponding to the positions of the amino acids at positions 5-12 or SEQ ID NO: 363 or 364 corresponding to the positions of the amino acids at positions 7-15 in SEQ ID NO: 185 are important for binding to IgE antibodies from allergic patients. Thus, in another preferred mode, when the antigen in spot 10 contains a variation in the amino acid sequence of SEQ ID NO: 70, the antigen may have an amino acid sequence of SEQ ID NO: 70 in which the amino acids at positions 5-12 of amino acids 1741-1755 are SEQ ID NO: 360 or 361 or the amino acids at positions 7-15 thereof are SEQ ID NO: 363 or 364.

### (11) Antigen in Spot 11

As the result of sequence identification of the antigen in spot 11 by mass spectroscopy, the amino acid sequences of SEQ ID NOs: 111-119 were detected.

Also, the mass spectroscopic data (SEQ ID NOs: 111-119) obtained for spot 11 on a mass spectrometer was analyzed by comparing the data against the NCBI protein data, and as a result, glycogen phosphorylase, muscle form-like (amino acid sequence: SEQ ID NO: 110, encoding nucleotide sequence: SEQ ID NO: 109) was identified. SEQ ID NO: 111 corresponds to amino acids 471-479 of SEQ ID NO: 110, SEQ ID NO: 112 corresponds to amino acids 547-555 of SEQ ID NO: 110, SEQ ID NO: 113 corresponds to amino acids 203-215 of SEQ ID NO: 110, SEQ ID NO: 114 corresponds to amino acids 727-741 of SEQ ID NO: 110, SEQ ID NO: 115 corresponds to amino acids 508-520 of SEQ ID NO: 110, SEQ ID NO: 116 corresponds to amino acids 742-755 of SEQ ID NO: 110, SEQ ID NO: 117 corresponds to amino acids 13-29 of SEQ ID NO: 110, SEQ ID NO: 118 corresponds to amino acids 775-784 of SEQ ID NO: 110, and SEQ ID NO: 119 corresponds to amino acids 643-656 of SEQ ID NO: 110.

Accordingly, the antigens in spot 11 in the present invention can be any of below in (11A-a) to (11A-e) and (11B):
(11A-a) a protein comprising an amino acid sequence with deletion, substitution, insertion or addition of one or several amino acids in SEQ ID NO: 110;
(11A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 110;
(11A-c) a protein comprising an amino acid sequence encoded by a nucleotide sequence with deletion, substitution, insertion or addition of one or several nucleotides in SEQ ID NO: 109;
(11A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 109;
(11A-e) a protein comprising an amino acid sequence encoded by a nucleic acid that hybridizes under stringent conditions with a nucleic acid having a nucleotide sequence complementary to the nucleotide sequence of SEQ ID NO: 109;
(11B) a protein comprising at least one amino acid sequence selected from the group consisting of SEQ ID NOs: 110-119, preferably a protein comprising at least 2, 3, 4, 5, 6, 7, 8, or 9 types or all sequences of the amino acid sequences. As referred to above, the amino acid sequence of any of SEQ ID NOs: 110 to 119 may be an amino acid sequence derived therefrom by deletion, substitution, insertion or addition of one or several amino acids.

The proteins as defined above in (11A-a) to (11A-e) and (11B) include those proteins whose amino acid residues are modified by phosphorylation, sugar chain modification, aminoacylation, ring-opening, deamination or the like.

The proteins as defined above in (11A-a) to (11A-e) and (11B) can be proteins that are found in a protein spot with a molecular weight of around 80 kDa to 160 kDa, preferably around 80 kDa to 110 kDa, more preferably around 90 kDa to 110 kDa and an isoelectric point of 4.0 to 8.0, preferably 5.0 to 7.5, more preferably 6.5 to 7.0 on gels used in the two-dimensional electrophoresis performed under the conditions described above in the subsection titled “Identification of antigens”.

Preferably, a protein that is an antigen in spot 11 is an antigen of an allergy to fish.

Epitopes of the aforementioned antigens (11A-a) to (11A-e) and (11B) reside in amino acids 261-275 (SEQ ID NO: 186) of SEQ ID NO: 110. When the antigen in spot 11 contains a variation in the amino acid sequence of SEQ ID NO: 110, amino acids corresponding to this epitope may retain the sequence in SEQ ID NO: 110.

From the viewpoint that binding activity against IgE antibodies such as sensitivity or specificity remains even if the amino acid sequence is varied, the antigen in spot 11 may be the following variant.

In the epitope having the sequence of SEQ ID NO: 186, sites of amino acids other than X in SEQ ID NO: 366 corresponding to the positions of the amino acids at positions 1-10 or SEQ ID NO: 368 or 369 corresponding to the positions of the amino acids at positions 11-15 in SEQ ID NO: 186 are important for binding to IgE antibodies from allergic patients. Thus, in another preferred mode, when the antigen in spot 11 contains a variation in the amino acid sequence of SEQ ID NO: 110, the antigen may have an amino acid sequence of SEQ ID NO: 110 in which the amino acids at positions 1-10 of amino acids 261-275 are SEQ ID NO: 366 or the amino acids at positions 11-15 thereof are SEQ ID NO: 368 or 369.

### (12) Antigen in Spot 12

As the result of sequence identification of the antigen in spot 12 by mass spectroscopy, the amino acid sequences of SEQ ID NOs: 122-136 were detected.

Also, the mass spectroscopic data (SEQ ID NOs: 122-136) obtained for spot 12 on a mass spectrometer was analyzed by comparing the data against the NCBI protein data, and as a result, myosin-binding protein C, fast-type-like (amino acid sequence: SEQ ID NO: 121, encoding nucleotide sequence: SEQ ID NO: 120) was identified. SEQ ID NO: 122 corresponds to amino acids 197-204 of SEQ ID NO: 121, SEQ ID NO: 123 corresponds to amino acids 421-434 of SEQ ID NO: 121, SEQ ID NO: 124 corresponds to amino acids 329-336 of SEQ ID NO: 121, SEQ ID NO: 125 corresponds to amino acids 413-420 of SEQ ID NO: 121, SEQ ID NO: 126 corresponds to amino acids 240-246 of SEQ ID NO: 121, SEQ ID NO: 127 corresponds to amino acids 214-228 of SEQ ID NO: 121, SEQ ID NO: 128 corresponds to amino acids 1014-1024 of SEQ ID NO: 121, SEQ ID NO: 129 corresponds to amino acids 842-855 of SEQ ID NO: 121, SEQ ID NO: 130 corresponds to amino acids 260-274 of SEQ ID NO: 121, SEQ ID NO: 131 corresponds to amino acids 672-685 of SEQ ID NO: 121, SEQ ID NO: 132 corresponds to amino acids 506-512 of SEQ ID NO: 121, SEQ ID NO: 133 corresponds to amino acids 205-213 of SEQ ID NO: 121, SEQ ID NO: 134 corresponds to amino acids 165-182 of SEQ ID NO: 121, SEQ ID NO: 135 corresponds to amino acids 321-328 of SEQ ID NO: 121, and SEQ ID NO: 136 corresponds to amino acids 70-82 of SEQ ID NO: 121.

Accordingly, the antigen in spot 12 in the present invention can be any of below in (12A-a) to (12A-e), and (12B):
(12A-a) a protein comprising an amino acid sequence with deletion, substitution, insertion or addition of one or several amino acids in SEQ ID NO: 121;
(12A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 121;
(12A-c) a protein comprising an amino acid sequence encoded by a nucleotide sequence with deletion, substitution, insertion or addition of one or several nucleotides in SEQ ID NO: 119;
(12A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 119;
(12A-e) a protein comprising an amino acid sequence encoded by a nucleic acid that hybridizes under stringent conditions with a nucleic acid having a nucleotide sequence complementary to the nucleotide sequence of SEQ ID NO: 119;
(12B) a protein comprising at least one amino acid sequence selected from the group consisting of SEQ ID NOs: 121-136, preferably a protein comprising at least 2, 3, 4, 5, 6, 7, 8, 9, 10, 11, 12, 13, 14, or 15 types or all sequences of the amino acid sequences. As referred to above, the amino acid sequence of any of SEQ ID NOs: 121 to 136 may be an amino acid sequence derived therefrom by deletion, substitution, insertion or addition of one or several amino acids.

The proteins as defined above in (12A-a) to (12A-e) and (12B) include those proteins whose amino acid residues are modified by phosphorylation, sugar chain modification, aminoacylation, ring-opening, deamination or the like.

The proteins as defined above in (12A-a) to (12A-e) and (12B) can be proteins that are found in a protein spot with a molecular weight of around 100 kDa to 160 kDa, preferably around 110 kDa to 150 kDa, more preferably around 120 kDa to 140 kDa and an isoelectric point of 4.0 to 7.0, preferably 4.0 to 6.0, more preferably 5.0 to 6.0 on gels used in the two-dimensional electrophoresis performed under the conditions described above in the subsection titled “Identification of antigens”.

Preferably, a protein that is an antigen in spot 12 is an antigen of an allergy to fish.

Epitopes of the aforementioned antigens (12A-a) to (12A-e) and (12B) reside in amino acids 181-195 (SEQ ID NO: 187), amino acids 211-225 (SEQ ID NO: 188), amino acids 221-235 (SEQ ID NO: 189), amino acids 231-245 (SEQ ID NO: 190), amino acids 251-265 (SEQ ID NO: 191), amino acids 371-385 (SEQ ID NO: 192), amino acids 491-505 (SEQ ID NO: 193), amino acids 651-665 (SEQ ID NO: 194), amino acids 831-845 (SEQ ID NO: 195), and amino acids 951-965 (SEQ ID NO: 196) of SEQ ID NO: 121. When the antigen in spot 12 contains a variation in the amino acid sequence of SEQ ID NO: 121, amino acids corresponding to at least one of these epitopes may retain the sequence in SEQ ID NO: 121.

From the viewpoint that binding activity against IgE antibodies such as sensitivity or specificity remains even if the amino acid sequence is varied, the antigen in spot 12 may be the following variant.

In the epitope having the sequence of SEQ ID NO: 187, sites of amino acids other than X in SEQ ID NO: 371 or 372 corresponding to the positions of the amino acids at positions 2-11 or SEQ ID NO: 374 or 375 corresponding to the positions of the amino acids at positions 7-14 in SEQ ID NO: 187 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 2-11 of amino acids 181-195 are SEQ ID NO: 371 or 372 or the amino acids at positions 7-14 thereof are SEQ ID NO: 374 or 375.

In the epitope having the sequence of SEQ ID NO: 188, sites of amino acids other than X in SEQ ID NO: 377 or 378 corresponding to the positions of the amino acids at positions 1-10 or SEQ ID NO: 380 or 381 corresponding to the positions of the amino acids at positions 8-15 in SEQ ID NO: 188 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 1-10 of amino acids 211-225 are SEQ ID NO: 377 or 378 or the amino acids at positions 8-15 thereof are SEQ ID NO: 380 or 381.

In the epitope having the sequence of SEQ ID NO: 189, sites of amino acids other than X in SEQ ID NO: 383 corresponding to the positions of the amino acids at positions 2-8 in SEQ ID NO: 189 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 2-8 of amino acids 221-235 are SEQ ID NO: 383.

In the epitope having the sequence of SEQ ID NO: 190, sites of amino acids other than X in SEQ ID NO: 385 corresponding to the positions of the amino acids at positions 2-9, SEQ ID NO: 387 corresponding to the positions of the amino acids at positions 1-10, or SEQ ID NO: 389 corresponding to the positions of the amino acids at positions 9-15 in SEQ ID NO: 190 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 2-9 of amino acids 231-245 are SEQ ID NO: 385, the amino acids at positions 1-10 thereof are SEQ ID NO: 387, or the amino acids at positions 9-15 thereof are SEQ ID NO: 389.

In the epitope having the sequence of SEQ ID NO: 191, sites of amino acids other than X in SEQ ID NO: 391 corresponding to the positions of the amino acids at positions 6-11 or SEQ ID NO: 393 or 394 corresponding to the positions of the amino acids at positions 6-12 in SEQ ID NO: 191 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 6-11 of amino acids 251-265 are SEQ ID NO: 391 or the amino acids at positions 6-12 thereof are SEQ ID NO: 393 or 394.

In the epitope having the sequence of SEQ ID NO: 192, sites of amino acids other than X in SEQ ID NO: 396 corresponding to the positions of the amino acids at positions 5-8 or SEQ ID NO: 398 or 399 corresponding to the positions of the amino acids at positions 7-15 in SEQ ID NO: 192 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 5-8 of amino acids 371-385 are SEQ ID NO: 396 or the amino acids at positions 7-15 thereof are SEQ ID NO: 398 or 399.

In the epitope having the sequence of SEQ ID NO: 193, sites of amino acids other than X in SEQ ID NO: 401 corresponding to the positions of the amino acids at positions 1-7 or SEQ ID NO: 403 or 404 corresponding to the positions of the amino acids at positions 6-15 in SEQ ID NO: 193 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 1-7 of amino acids 491-505 are SEQ ID NO: 401 or the amino acids at positions 6-15 thereof are SEQ ID NO: 403 or 404.

In the epitope having the sequence of SEQ ID NO: 194, sites of amino acids other than X in SEQ ID NO: 406 corresponding to the positions of the amino acids at positions 3-7 in SEQ ID NO: 194 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 3-7 of amino acids 651-665 are SEQ ID NO: 406.

In the epitope having the sequence of SEQ ID NO: 195, sites of amino acids other than X in SEQ ID NO: 408 corresponding to the positions of the amino acids at positions 2-13 in SEQ ID NO: 195 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 2-13 of amino acids 831-845 are SEQ ID NO: 408.

In the epitope having the sequence of SEQ ID NO: 196, sites of amino acids other than X in SEQ ID NO: 410 corresponding to the positions of the amino acids at positions 1-10 or SEQ ID NO: 412 corresponding to the positions of the amino acids at positions 1-8 in SEQ ID NO: 196 are important for binding to IgE antibodies from allergic patients. Thus, in another preferred mode, when the antigen in spot 12 contains a variation in the amino acid sequence of SEQ ID NO: 121, the antigen may have an amino acid sequence of SEQ ID NO: 121 in which the amino acids at positions 1-10 of amino acids 951-965 are SEQ ID NO: 410 or the amino acids at positions 1-8 thereof are SEQ ID NO: 412.

### (13) Antigen in Spot 13

As the result of sequence identification of the antigen in spot 13 by mass spectroscopy, the amino acid sequences of SEQ ID NOs: 139-142 were detected.

Also, the mass spectroscopic data (SEQ ID NOs: 139-142) obtained for spot 13 on a mass spectrometer was analyzed by comparing the data against the NCBI protein data, and as a result, ATP synthase subunit beta, mitochondrial (amino acid sequence: SEQ ID NO: 138, encoding nucleotide sequence: SEQ ID NO: 137) was identified. SEQ ID NO: 139 corresponds to amino acids 191-201 of SEQ ID NO: 138, SEQ ID NO: 140 corresponds to amino acids 449-469 of SEQ ID NO: 138, SEQ ID NO: 141 corresponds to amino acids 202-214 of SEQ ID NO: 138, and SEQ ID NO: 142 corresponds to amino acids 178-187 of SEQ ID NO: 138.

Accordingly, the antigen in spot 13 in the present invention can be any of the proteins as defined below in (13A-a) to (13A-e) and (13B).
(13A-a) a protein comprising an amino acid sequence with deletion, substitution, insertion or addition of one or several amino acids in SEQ ID NO: 138;
(13A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 138;
(13A-c) a protein comprising an amino acid sequence encoded by a nucleotide sequence with deletion, substitution, insertion or addition of one or several nucleotides in SEQ ID NO: 137;
(13A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 137;
(13A-e) a protein comprising an amino acid sequence encoded by a nucleic acid that hybridizes under stringent conditions with a nucleic acid having a nucleotide sequence complementary to the nucleotide sequence of SEQ ID NO: 137;
(13B) a protein comprising at least one amino acid sequence selected from the group consisting of SEQ ID NOs: 138-142, preferably a protein comprising at least 2, 3, or 4 or all sequences of the amino acid sequences. As referred to above, the amino acid sequence of any of SEQ ID NOs: 138 to 142 may be an amino acid sequence derived therefrom by deletion, substitution, insertion or addition of one or several amino acids.

The proteins as defined above in (13A-a) to (13A-e) and (13B) include those proteins whose amino acid residues are modified by phosphorylation, sugar chain modification, aminoacylation, ring-opening, deamination or the like.

The proteins as defined above in (13A-a) to (13A-e) and (13B) can be proteins that are found in a protein spot with a molecular weight of around 30 kDa to 70 kDa, preferably around 40 kDa to 60 kDa, more preferably around 45 kDa to 55 kDa and an isoelectric point of 3.0 to 7.0, preferably 3.0 to 6.0, more preferably 4.0 to 5.5 on gels used in the two-dimensional electrophoresis performed under the conditions described above in the subsection titled “Identification of antigens”.

Preferably, a protein that is an antigen in spot 13 is an antigen of an allergy to fish.

Epitopes of the aforementioned antigens (13A-a) to (13A-e) and (13B) reside in amino acids 211-225 (SEQ ID NO: 197) of SEQ ID NO: 138. When the antigen in spot 13 contains a variation in the amino acid sequence of SEQ ID NO: 138, amino acids corresponding to this epitope may retain the sequence in SEQ ID NO: 138.

From the viewpoint that binding activity against IgE antibodies such as sensitivity or specificity remains even if the amino acid sequence is varied, the antigen in spot 13 may be the following variant.

In the epitope having the sequence of SEQ ID NO: 197, sites of amino acids other than X in SEQ ID NO: 414 corresponding to the positions of the amino acids at positions 7-10 or SEQ ID NO: 416 corresponding to the positions of the amino acids at positions 9-14 in SEQ ID NO: 197 are important for binding to IgE antibodies from allergic patients. Thus, in another preferred mode, when the antigen in spot 13 contains a variation in the amino acid sequence of SEQ ID NO: 138, the antigen may have an amino acid sequence of SEQ ID NO: 138 in which the amino acids at positions 7-10 of amino acids 211-225 are SEQ ID NO: 414 or the amino acids at positions 9-14 thereof are SEQ ID NO: 416.

### (14) Antigen in Spot 14

As the result of sequence identification of the antigen in spot 14 by mass spectroscopy, the amino acid sequences of SEQ ID NOs: 145-149 were detected.

Also, the mass spectroscopic data (SEQ ID NOs: 145-149) obtained for spot 14 on a mass spectrometer was analyzed by comparing the data against the NCBI protein data, and as a result, L-lactate dehydrogenase A chain-like (amino acid sequence: SEQ ID NO: 144, encoding nucleotide sequence: SEQ ID NO: 143) was identified. SEQ ID NO: 145 corresponds to amino acids 119-126 of SEQ ID NO: 144, SEQ ID NO: 146 corresponds to amino acids 7-22 of SEQ ID NO: 144, SEQ ID NO: 147 corresponds to amino acids 9-22 of SEQ ID NO: 144, SEQ ID NO: 148 corresponds to amino acids 270-278 of SEQ ID NO: 144, and SEQ ID NO: 149 corresponds to amino acids 91-118 of SEQ ID NO: 144.

Accordingly, the antigen in spot 14 in the present invention can be any of the proteins as defined below in (14A-a) to (14A-e) and (14B).
(14A-a) a protein comprising an amino acid sequence with deletion, substitution, insertion or addition of one or several amino acids in SEQ ID NO: 144;
(14A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 144;
(14A-c) a protein comprising an amino acid sequence encoded by a nucleotide sequence with deletion, substitution, insertion or addition of one or several nucleotides in SEQ ID NO: 143;
(14A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 143;
(14A-e) a protein comprising an amino acid sequence encoded by a nucleic acid that hybridizes under stringent conditions with a nucleic acid having a nucleotide sequence complementary to the nucleotide sequence of SEQ ID NO: 143;
(14B) a protein comprising at least one amino acid sequence selected from the group consisting of SEQ ID NOs: 144-149, preferably a protein comprising at least 2, 3, 4, or 5 or all sequences of the amino acid sequences. As referred to above, the amino acid sequence of any of SEQ ID NOs: 144 to 149 may be an amino acid sequence derived therefrom by deletion, substitution, insertion or addition of one or several amino acids.

The proteins as defined above in (14A-a) to (14A-e) and (14B) include those proteins whose amino acid residues are modified by phosphorylation, sugar chain modification, aminoacylation, ring-opening, deamination or the like.

The proteins as defined above in (14A-a) to (14A-e) and (14B) can be proteins that are found in a protein spot with a molecular weight of around 20 kDa to 50 kDa, preferably around 30 kDa to 50 kDa, more preferably around 30 kDa to 40 kDa and an isoelectric point of 5.0 to 9.0, preferably 6.0 to 8.0, more preferably 6.5 to 7.5 on gels used in the two-dimensional electrophoresis performed under the conditions described above in the subsection titled “Identification of antigens”.

Preferably, a protein that is an antigen in spot 14 is an antigen of an allergy to fish.

Epitopes of the aforementioned antigens (14A-a) to (14A-e) and (14B) reside in amino acids 11-25 (SEQ ID NO: 198) of SEQ ID NO: 144. When the antigen in spot 14 contains a variation in the amino acid sequence of SEQ ID NO: 144, amino acids corresponding to this epitope may retain the sequence in SEQ ID NO: 144.

From the viewpoint that binding activity against IgE antibodies such as sensitivity or specificity remains even if the amino acid sequence is varied, the antigen in spot 14 may be the following variant.

In the epitope having the sequence of SEQ ID NO: 198, sites of amino acids other than X in SEQ ID NO: 418 or 419 corresponding to the positions of the amino acids at positions 5-13 in SEQ ID NO: 198 are important for binding to IgE antibodies from allergic patients. Thus, in another preferred mode, when the antigen in spot 14 contains a variation in the amino acid sequence of SEQ ID NO: 144, the antigen may have an amino acid sequence of SEQ ID NO: 144 in which the amino acids at positions 5-13 of amino acids 11-25 are SEQ ID NO: 418 or 419.

By stating herein “deletion, substitution, insertion or addition of one or several amino acids” in relation to amino acid sequence, it is meant that in an amino acid sequence of interest, one or several amino acids (e.g., 30%, preferably 25%, 20%, 15%, 10%, 5%, 3%, 2% or 1%, of amino acids with respect to the total length of the amino acid sequence) are deleted, one or several amino acids are substituted by any other amino acids, any other amino acids are inserted, and/or any other amino acids are added.

Among the aforementioned modifications, substitution is preferably conservative substitution. The “conservative substitution” refers to the substitution of a certain amino acid residue by a different amino acid residue having similar physicochemical characteristics, and can be any type of substitution as long as it does not substantially change the characteristics of the structure of the original sequence for example, any type of substitution is acceptable as long as any substituted amino acids do not disrupt the helical structure of the original sequence or other secondary structures that characterize the original sequence. The following gives examples of separate groups of amino acid residues that are conservatively substitutable with each other, but substitutable amino acid residues are not limited to the examples given below.
Group A: leucine, isoleucine, valine, alanine, methionine
Group B: aspartic acid, glutamic acid
Group C: asparagine, glutamine
Group D: lysine, arginine
Group E: serine, threonine
Group F: phenylalanine, tyrosine

In the case of non-conservative substitution, one member belonging to one of the aforementioned groups can be replaced with a member belong to any other group. For example, in order to eliminate the possibility of unwanted sugar-chain modification, amino acids of group B, D or E as listed above may be substituted by those of any other group. Also, cysteines may be deleted or substituted by any other amino acids to prevent them from being folded into a protein in its tertiary structure. Also, in order to maintain the balance between hydrophilicity and hydrophobicity or to increase hydrophilicity for the purpose of facilitating synthesis, any amino acids may be substituted in consideration of the hydropathy scales of amino acids, which are a measure of the hydrophilic and hydrophobic properties of amino acids (J. Kyte and R. Doolittle, J. Mol. Biol., Vol. 157, p. 105-132, 1982).

In another mode, substitution of the original amino acid by an amino acid with less steric hindrance, for example, substitution of group F by group A, B, C, D or E; or substitution of an amino acid having an electric charge by an amino acid having no electric charge, for example, substitution of group B by group C, may be performed. This may improve binding activity against IgE antibodies.

As referred to herein, the percent identity between two amino acid sequences can be determined by visual inspection and mathematical calculation. Alternatively, the percent identity can be determined using a computer program. Examples of such computer programs include BLAST and ClustalW. In particular, various conditions (parameters) for identity searches with the BLAST program are described in Altschul, et al. (Nucl. Acids. Res., 25, p. 3389-3402, 1997), and are publicly available on the websites of the National Center for Biotechnology Information (NCBI) and DNA Data Bank of Japan (DDBJ) (Altschul, et al., BLAST Handbook, Altschul, et al., NCB/NLM/NIH Bethesda, MD 20894). Also, the percent identity can be determined using a genetic information processing software program, such as GENETYX Ver. 7 (Genetyx Corporation), DINASIS Pro (Hitachi Software Engineering Co., Ltd.), or Vector NTI (Infomax Inc.).

By stating herein “deletion, substitution, insertion or addition of one or several nucleotides” in relation to nucleotide sequence, it is meant that in a nucleotide sequence of interest, one or several nucleotides (e.g., 30%, preferably 25%, 20%, 15%, 10%, 5%, 3%, 2% or 1%, of amino acid with respect to the total length of the nucleotide sequence, or e.g., 1, 5, 10, 15, 20, 25 or 30 nucleotides) are deleted, one or several nucleotides are substituted by any other nucleotides, any other nucleotides are inserted, and/or any other nucleotides are added. It is preferable that such a nucleotide deletion, substitution, insertion or addition should not give rise to a frame shift in an amino acid coding sequence.

As referred to herein, the percent identity between two nucleotide sequences can be determined by visual inspection and mathematical calculation. Alternatively, the percent identity can be determined using a computer program. Examples of such sequence comparison computer programs include the BLASTN program, version 2.2.7, available on the website of the National Library of Medicine (http://www.ncbi.nlm.nih.gov/blast/bl2seq/bls.html) (Altschul, et al. (1990) J. Mol. Biol., 215: 403-10), or the WU-BLAST 2.0 algorithm. Standard default parameter settings for WU-BLAST 2.0 are found and available on the following website: http://blast.wustl.edu.

As referred to herein, “under stringent conditions” means that hybridization takes place under moderately or highly stringent conditions. To be specific, the moderately stringent conditions can be easily determined by those having ordinary skill in the art on the basis of, for example, the length of DNA. Basic conditions are described in Sambrook, et al., Molecular Cloning: A Laboratory Manual, 3rd ed., ch. 6-7, Cold Spring Harbor Laboratory Press, 2001. The moderately stringent conditions include hybridization under the conditions of preferably 1×SSC to 6×SSC at 42° C. to 55° C., more preferably 1×SSC to 3×SSC at 45° C. to 50° C., most preferably 2×SSC at 50° C. In the case of using a hybridization solution containing, for example, about 50% formamide, a temperature around 5 to 15° C. lower than the foregoing should be adopted. Washing is also carried out under the conditions of 0.5×SSC to 6×SSC at 40° C. to 60° C. In the process of hybridization and washing, generally 0.05% to 0.2% SDS, preferably about 0.1% SDS, may be added. Likewise, the highly stringent conditions can be easily determined by those having ordinary skill in the art on the basis of, for example, the length of DNA. Generally, the highly stringent (high stringent) conditions include hybridization and/or washing at a higher temperature and/or a lower salt concentration than those adopted under the moderately stringent conditions. For example, hybridization is carried out under the conditions of 0.1×SSC to 2×SSC at 55° C. to 65° C., more preferably 0.1×SSC to 1×SSC at 60° C. to 65° C., most preferably 0.2×SSC at 63° C. Washing is carried out under the conditions of 0.2×SSC to 2×SSC at 50° C. to 68° C., more preferably 0.2×SSC at 60 to 65° C.

Antigens may be obtained by separating and purifying them from fish using a combination of protein purification methods well known to those skilled in the art. Also, antigens may be obtained by expressing them as recombinant proteins using a genetic recombination technique well known to those skilled in the art and by separating and purifying them using protein purification methods well known to those skilled in the art.

Exemplary protein purification methods include: solubility-based purification methods such as salt precipitation and solvent precipitation; purification methods based on molecular weight difference, such as dialysis, ultrafiltration, gel filtration and SDS-PAGE; charge-based purification methods such as ion exchange chromatography and hydroxylapatite chromatography; specific affinity-based purification methods such as affinity chromatography; purification methods based on hydrophobicity difference, such as reverse-phase high-performance liquid chromatography; and purification methods based on isoelectric point difference, such as isoelectric focusing.

Preparation of a protein by a genetic recombination technique is carried out by preparing an expression vector comprising an antigen-encoding nucleic acid, introducing the expression vector into appropriate host cells by gene transfer or genetic transformation, culturing the host cells under suitable conditions for expression of a recombinant protein, and recovering the recombinant protein expressed in the host cells.

The “vector” refers to a nucleic acid that can be used to introduce a nucleic acid attached thereto into host cells. The “expression vector” is a vector that can induce the expression of a protein encoded by a nucleic acid introduced therethrough. Exemplary vectors include plasmid vectors and viral vectors. Those skilled in the art can select an appropriate expression vector for the expression of a recombinant protein depending on the type of host cells to be used. In order to facilitate purification, an affinity tag such as His×6 residues may be contained therein. Furthermore, the vector may be synthesized such that a protein containing a signal sequence is secreted from cells.

The “host cells” refers to cells that undergo gene transfer or genetic transformation by a vector. The host cells can be appropriately selected by those skilled in the art depending on the type of the vector to be used. The host cells can be derived from prokaryotes such as E. coli. When prokaryotic cells like E. coli are used as host cells, the antigen of the present invention may be designed to contain an N-terminal methionine residue in order to facilitate the expression of a recombinant protein in the prokaryotic cells. The N-terminal methionine can be cleaved from the recombinant protein after expression. Also, the host cells may be cells derived from eukaryotes, such as single-cell eukaryotes like yeast, plant cells and animal cells (e.g., human cells, monkey cells, hamster cells, rat cells, murine cells or insect cells) or silkworm.

Gene transfer or genetic transformation of an expression vector into host cells can be carried out as appropriate by following a technique known to those skilled in the art. Those skilled in the art can make possible the expression of a recombinant protein by selecting suitable conditions for the expression of the recombinant protein as appropriate depending on the type of host cells and culturing the host cells under the selected conditions. Then, those skilled in the art can homogenize the host cells having the expressed recombinant protein, and separate and purify an antigen expressed as the recombinant protein from the resulting homogenate by using an appropriate combination of such protein purification methods as mentioned above.

### Diagnosis Kit and Method (1)

The present invention provides a method for providing an indicator for diagnosing an allergy to fish in a subject, the method comprising the steps of:
(i) contacting a sample obtained from the subject with an antigen, wherein the sample is a solution comprising an IgE antibody;
(ii) detecting binding between an IgE antibody present in the sample from the subject and the antigen; and
(iii) when the binding between the IgE antibody in the subject and the antigen is detected, an indicator of the fact that the subject is allergic to fish is provided;
wherein the antigen is at least one of proteins as defined above in any of (10) to (14).

The sample obtained from a subject is a solution containing an IgE antibody, as collected from the subject. Examples of such solutions include blood, saliva, sputum, snivel, urine, sweat, and tear. The sample obtained from the subject may be subjected to pretreatment for increasing the concentration of an IgE antibody in the sample before being contacted with an antigen. The pretreatment of a sample may involve, for example, collection of the serum or the plasma from the blood. Furthermore, a Fab moiety that is an antigen-binding moiety may be purified. In a particularly preferred mode, the step (i) mentioned above is carried out by contacting an IgE antibody present in the serum obtained from a subject with an antigen.

The IgE antibody may be the IgE antibody itself or may be mast cells or the like bound to IgE antibodies.

Detection of contact and binding between the sample obtained from a subject and an antigen can be carried out by using a known method. Examples of such methods that can be used include detection by ELISA (Enzyme-Linked Immunosorbent Assay), sandwich immunoassay, immunoblotting, immunoprecipitation, or immunochromatography. These are all techniques for contacting and binding an IgE antibody from a subject with an antigen, and detecting the IgE antibody specifically bound to the antigen. Examples thereof include a method for allowing an enzymatically labelled secondary antibody to act on the IgE antibody specifically bound to the antigen, and adding an enzyme substrate (generally, chromogenic or luminescent reagent) to detect an enzymatic reaction product, a method for allowing a biotin-labeled secondary antibody to act on the IgE antibody specifically bound to the antigen, adding an avidin-bound dye, and detecting the dye, and a method for detecting a fluorescently labeled secondary antibody. Alternatively, detection by a measurement method capable of evaluating binding between an antigen and an IgE antibody, such as surface plasmon resonance (SPR), can also be used. A plurality of antigen-specific IgE antibodies may be mixed.

The antigen may be provided as an isolated antigen in a state immobilized on a carrier. In this case, the steps (i) and (ii) mentioned above can be carried out using ELISA, sandwich immunoassay, immunochromatography, surface plasmon resonance, or the like. Also, the step (i) mentioned above can be carried out by contacting the sample obtained from a subject with an antigen-immobilized surface. The isolated antigen may be obtained by separating and purifying it from fish using a combination of protein purification methods well known to those skilled in the art, or by preparing it using a genetic recombination technique. Alternatively, the antibody to bind to the antigen may be used in a state immobilized on a carrier.

The antigen may be in a state unimmobilized on a carrier. In this case, flow cytometry or the like can be used in the aforementioned steps (i) and (ii), and the presence of the antigen bound to the antibody can be confirmed with laser beam. Examples of this method include a basophil activation test (BAT) which is a method in which the amount of a surface antigen CD203c that appears when basophils in a sample are activated by the binding of the antigen to an antibody is measured. Another example includes a histamine release test (HRT) which examines whether histamine is released by contacting the antigen with blood cells through binding to an antibody in a sample.

The antigen may be detected by immunoblotting method by transferring from a state separated by two-dimensional electrophoresis. The two-dimensional electrophoresis is a technique for separating a protein sample by performing isoelectric focusing in the first dimension and performing SDS-PAGE (SDS-polyacrylamide gel electrophoresis) in the second dimension. The conditions for two-dimensional electrophoresis are not particularly limited as long as the conditions permit the separation of the antigen of the present invention. For example, the conditions for two-dimensional electrophoresis as described above in the subsection titled “Identification of antigens” can be adopted. Also, the electrophoresis conditions may be defined by reference to the descriptions in Patent Literatures 1 to 4 mentioned above. For example, two-dimensional electrophoresis can be carried out under the conditions that satisfy at least one selected from the group consisting of the following requirements:
(A) the isoelectric focusing gels used in the first dimension should have a gel-strip length of 5 to 10 cm and a gel pH range of 3 to 10, and the pH gradient of the gels in the direction of electrophoresis should be as follows: where the gel-strip length up to pH 5 is taken as “a”, that length from pH 5 to 7 as “b”, and that length above pH 7 as “c”, the relations “a<b” and “b>c” are satisfied;
(B) in the case of (A), when the total gel-strip length is taken as 1, “a” should be in the range of 0.15 to 0.3, “b” should be in the range of 0.4 to 0.7, and “c” should be in the range of 0.15 to 0.3;
(C) in the first dimensional isoelectric focusing, a constant voltage step should be performed by applying a constant voltage ranging from 100 V to 600 V per gel strip containing a sample, and after the electrophoresis variation width during electrophoresis for 30 minutes falls within the range of 5 μA, a voltage-increasing step should be started at which the voltage is increased from the aforementioned constant voltage;
(D) in the case of (C), the final voltage at the voltage-increasing step should be in the range of 3000 V to 6000 V;
(E) the isoelectric focusing gels used in the first dimension should have a longitudinal gel-strip length of 5 to 10 cm, and the electrophoresis gels used in the second dimension should have a gel concentration at the distal end in the direction of electrophoresis, which is in the range of 3 to 6%; and
(F) in the case of (E), the electrophoresis gels used in the second dimension should have a gel concentration at the proximal end in the direction of electrophoresis, which is set to a higher value than that at the distal end in the direction of electrophoresis.

The aforementioned antigens (10) to (14) are antigens that specifically bind to IgE antibodies from patients with allergy to fish. Therefore, when binding between an IgE antibody from a subject and the antigen is detected, an indicator of the fact that the subject is allergic to fish is provided.

The present invention further provides a kit for diagnosing an allergy to fish, comprising at least one of the aforementioned antigens (10) to (14). The diagnosis kit of this invention may be used in the aforementioned method for providing an indicator for diagnosing an allergy to fish or in a diagnosis method as described later. The diagnosis kit of this invention may comprise not only the at least one of the aforementioned antigens (10) to (14), but also an anti-IgE antibody labeled with an enzyme and a chromogenic or luminescent substrate serving as a substrate for the enzyme, or an anti-IgE antibody labeled with biotin and an avidin-bound dye binding to the biotin. Also, a fluorescent-labeled anti-IgE antibody may be used. In the diagnosis kit of this invention, the antigen may be provided in a state immobilized on a carrier. The diagnosis kit of this invention may also be provided together with instructions on the procedure for diagnosis or a package containing said instructions.

In another mode, the aforementioned diagnosis kit comprises a companion diagnostic agent for an allergy to fish. The companion diagnostic agent is used for identifying patients expected to respond to pharmaceutical products or identifying patients having the risk of severe adverse reactions to pharmaceutical products, or for studying the reactivity of pharmaceutical products in order to optimize treatment using the pharmaceutical products. Here, the optimization of treatment includes, for example, determination of dosage and administration, judgment regarding discontinuation of administration, and confirmation of an allergen component that is used to cause immunological tolerance.

The present invention further provides a composition for diagnosing an allergy to fish, comprising at least one of the aforementioned antigens (10) to (14). The diagnosis composition of this invention can be used in a diagnosis method as described below. The diagnosis composition of this invention may further comprise a pharmaceutically acceptable carrier and/or additives commonly used with the antigen of this invention depending on the need.

In one mode, the present invention provides a method for diagnosing an allergy to fish in a subject, the method comprising:
(i) contacting a sample obtained from the subject with an antigen;
(ii) detecting binding between an IgE antibody present in the sample from the subject and the antigen; and
(iii) when the binding between the IgE antibody in the subject and the antigen is detected, diagnosing the subject as being allergic to fish;
wherein the antigen is at least one of proteins as defined above in any of (10) to (14). In this method, the steps (i) and (ii) are performed as described above regarding the corresponding steps of the method for providing an indicator for diagnosing an allergy to fish.

In another mode, the present invention provides a method for diagnosing an allergy to fish in a subject, the method comprising administering to the subject at least one of the aforementioned antigens (10) to (14). This method may be performed in the form of a skin test characterized by applying the antigen onto the skin. Examples of the skin test include various forms of tests, such as: a prick test in which a diagnosis composition is applied onto the skin and then a tiny prick to such an extent as not to provoke bleeding is made in the skin to allow an antigen to penetrate the skin, thereby observing a skin reaction; a scratch test in which a diagnosis composition is applied onto the skin and then the skin is lightly scratched to observe a reaction; a patch test in which a diagnosis composition in the form of cream, ointment, etc. is applied onto the skin to observe a reaction; and an intracutaneous test in which an antigen is administered intracutaneously to observe a reaction. The method for allowing the diagnosis composition to penetrate the skin in the prick test or the scratch test may be a method in which the tip of a lancet is contacted with the diagnosis composition, and the contact site is inserted to the skin so that a prick is made in the skin to allow the diagnosis composition to penetrate the skin. If a skin reaction such as swelling occurs in a skin portion to which the antigen has been applied, the subject of interest is diagnosed as having an allergy to fish. The amount of the antigen to be applied to the skin in such tests can be, for example, not more than 100 μg per dose.

In the process of allergy diagnosis, a load test aiming to identify an antigen is often adopted. At least one of the aforementioned antigens (10) to (14) can be used as an active ingredient for a load test to diagnose an allergy to fish. Here, the antigen protein to be used in the load test may be a protein that has been expressed and purified and may be a protein that has been expressed in food or cooking ingredients, such as rice-based vaccine expressing pollen allergens which are obtained by transforming rice with a gene of a cedar pollen antigen and expressing the antigen protein in the rice.

In yet another mode, the present invention provides at least one of the aforementioned antigens (10) to (14), intended for use in the diagnosis of an allergy to fish. This also includes the provision of at least one of the aforementioned antigens (10) to (14) mixed with a known antigen.

In still another mode, the present invention provides use of at least one of the aforementioned antigens (10) to (14) for the production of a diagnostic agent for an allergy to fish.

### Pharmaceutical Composition and Treatment Method (1)

The present invention provides a pharmaceutical composition comprising at least one of the aforementioned antigens (10) to (14).

In one mode, the aforementioned pharmaceutical composition is used for the treatment and diagnosis of an allergy to fish. The treatment of an allergy increases the limit amount of an antigen in which the allergy does not develop even if the antigen is incorporated into the body, and finally aims for the state where the allergy does not develop by the common amount of the antigen to be consumed (remission).

The present invention also provides a method for treating an allergy to fish, the method comprising administering at least one of the aforementioned antigens (10) to (14) to a patient in need of a treatment for an allergy to fish.

In another mode, the present invention provides at least one of the aforementioned antigens (10) to (14), intended for use in the treatment for an allergy to fish. In yet another mode, the present invention provides use of at least one of the aforementioned antigens (10) to (14) for the production of a therapeutic agent for an allergy to fish.

In the process of allergy treatment, a hyposensitization therapy aiming to induce immunological tolerance by administering an antigen to a patient is often adopted. The at least one of the aforementioned antigens (10) to (14) can be used as an active ingredient for a hyposensitization therapy for an allergy to fish. Here, the antigen protein to be used in the hyposensitization therapy may be a protein that has been expressed and purified and may be a protein that has been expressed in food or cooking ingredients, such as rice-based vaccine expressing pollen allergens which are obtained by transforming rice with a gene of a cedar pollen antigen and expressing the antigen protein in the rice.

The pharmaceutical composition of the present invention can be administered by common administration routes. Examples of common administration routes include oral, sublingual, percutaneous, intracutaneous, subcutaneous, intravascular, intranasal, intramuscular, intraperitoneal, and intrarectal administrations.

The pharmaceutical composition of the present invention can be used as a pharmaceutical composition to which a commonly used pharmaceutically acceptable adjuvant or excipient or any other additives (e.g., stabilizer, solubilizer, emulsifier, buffer, preservative, colorant) are added by a conventional method together with the antigen of this invention depending on the need. The dosage form of the pharmaceutical composition can be selected by those skilled in the art as appropriate depending on the administration route. The pharmaceutical composition can be in the form of, for example, tablet, capsule, troche, sublingual tablet, parenteral injection, intranasal spray, poultice, solution, cream, lotion, or suppository. The administration dose, frequency and/or period of the pharmaceutical composition of this invention can be selected by a physician as appropriate depending on the administration route and the patient's condition and characteristics such as age and body weight. For example, the pharmaceutical composition may be administered to an adult patient at a dose of not more than 100 μg per dose. The administration interval can be, for example, once daily, once weekly, twice weekly, once per three months or so. The administration period can be, for example, several weeks to several years. The pharmaceutical composition may be administered in such a manner that the dose is increased in incremental steps over the administration period.

### Tester (1)

The present invention provides a tester comprising an antibody for at least one of the aforementioned antigens (10) to (14).

The antibody can be prepared by a conventional method. For example, the antibody may be prepared by immunizing a mammal such as rabbit with one of the aforementioned antigens (10) to (14). The antibody may be an IgE antibody, a polyclonal antibody, a monoclonal antibody, or an antigen-binding fragment thereof (e.g., Fab, F(ab′)₂, Fab′).

Further, in the aforementioned tester, the antibody may be provided in a form bound to a carrier. The type of the carrier is not particularly limited as long as it is usable for detection of binding between an antibody and an antigen. Any given carrier known to those skilled in the art can be used.

Examples of a method for determining the presence or absence of an antigen include the following methods:
a method in which a prepared tester comprising an IgE antibody is contacted with a sample obtained from a food, a cooking ingredient, etc., ELISA or the like method is used to detect whether there is a binding between the IgE antibody and an antigen in the sample, and if the binding between the IgE antibody and the antigen is detected, it is determined that the antibody remains in the food, the cooking ingredient, etc. of interest;
a method in which proteins are extracted from a food or a cooking ingredient and electrophoresed, and the presence or absence of a spot band of an antigen is detected with an antibody; and
a method in which filter paper is impregnated with a food or a cooking ingredient and reacted with an antibody solution so as to detect an antigen contained therein.

Another mode of the present invention includes a tester for determining the presence or absence of an antigen of an allergy to fish in an object of interest, the tester comprising a primer having a nucleotide sequence complementary to a portion of at least one of the nucleotide sequences of SEQ ID NOs: 69, 109, 120, 137, and 143. The primer has, for example, but not limited to, a nucleotide sequence complementary to, preferably, 12 residues, 15 bases, 20 bases, or 25 bases, in a 3′-terminal portion or central sequence in a sequence of at least one of the nucleotide sequences of SEQ ID NOs: 69, 109, 120, 137, and 143. Particularly, when mRNA is of interest, the tester has a complementary primer of a poly-A tail. In a preferred mode, the tester comprising the primer mentioned above may further comprise a primer comprising a 5′-terminal nucleotide sequence, preferably a nucleotide sequence of 12 bases, 15 bases, 20 bases, or 25 bases, of at least one of the nucleotide sequences of SEQ ID NOs: 69, 109, 120, 137, and 143.

For example, DNA or cDNA is amplified by PCR (Polymerase Chain Reaction) including RT-PCR using templated DNA or mRNA obtained from a fish and the aforementioned primer, and the sequence of the amplified DNA or cDNA is compared with the nucleotide sequence of SEQ ID NO: 69, 109, 120, 137 or 143 to determine the presence or absence of the antigen. mRNA amplification methods by PCR can be exemplified by RACE. In this respect, even if there exists a point mutation encoding the same amino acid in the comparison of the amplified DNA or cDNA with the nucleotide sequence of SEQ ID NO: 69, 109, 120, 137 or 143, or even if the nucleotide sequence of the amplified DNA or cDNA has insertion, deletion, substitution or addition of bases in the nucleotide sequence of SEQ ID NO: 69, 109, 120, 137 or 143, it is determined that the antigen is present when the amino acid sequence encoded by the DNA or the cDNA has at least 70%, preferably at least 80, 90, 95, 98, or 99% identity to the amino acid sequence of SEQ ID NO: 70, 110, 121, 138 or 144.

In one mode, the aforementioned tester is used to determine the presence or absence of an antigen in cooking ingredients (a fish or a fish egg) or in products of interest in a food production line. The tester may also be used for quality inspection of production lines and pre-shipment products by manufacturers, or may be used for self-checking of the presence or absence of an antigen in a food or cooking ingredients of interest by consumers.

### Allergen-Free Food and the Like

The present invention provides a fish, fish egg or processed product of such fish or fish egg in which at least one of the aforementioned antigens (10) to (14) is eliminated or reduced, or a fish that delivers such fish egg or is born from such fish egg.

The method for eliminating or reducing the antigen of the present invention in a fish, fish egg or processed products of such fish or fish egg, a fish that delivers such fish egg or is born from such fish egg is not limited. The elimination or reduction of the antigen may be conducted by any method, as long as the method permits the elimination or reduction of the antigen.

For example, the fish or fish egg of the present invention whose antigen is eliminated or reduced may be obtained by preparing a fish or fish egg in which the expression of the antigen of the present invention is knocked out, using a gene knock-out technique.

Any technique known to those skilled in the art can be used as the gene knock-out technique. For example, Oishi, et al. (Scientific Reports, Vol. 6, Article number: 23980, 2016, doi:10.1038/srep23980) states that individuals with allergen protein gene deletion are obtained by applying a genome editing technique CRISPER/Cas9 to primordial germ cells. The fish or fish egg of the present invention whose antigen is eliminated may be obtained through the use of a similar technique. The fish or fish egg of the present invention whose antigen is eliminated or reduced may be obtained by mating through artificial insemination with fishes or fish eggs containing no antigen or containing the antigen in a small amount. The artificial crossing of fishes or fish eggs can be performed by a conventional method.

An antigen of the present invention may be the artefact that an antigen of the present invention assumed the removal or a reduced fish or fish egg raw material as for the removal or the reduced processed products of fish or fish egg. In the case of using an ordinary fish or fish egg as a source ingredient, a treatment for removing or reducing the antigen of this invention is performed before or after preparation of a processed product of fish or fish egg. The methods for removing or reducing the antigen of the present invention in the processed products of fish or fish egg which assumed an ordinary fish or fish egg raw material include a method to remove protein component in food or a cooking ingredient such as a high pressure treatment and elution with the neutral salt solution or the high temperature steam, and a method to perform hydrolysis, denaturation, or amino acid alteration (chemical modification, elimination, or the like of a side chain) by heat treatment and acid treatment. The fish of the present invention whose antigen is eliminated or reduced may be a fish grown from hatched fish eggs in which the aforementioned antigen of the present invention is eliminated or reduced. Also, the fish egg of the present invention whose antigen is eliminated or reduced may be obtained from fishes in which the aforementioned antigen of the present invention is eliminated or reduced.

### Method for Producing Allergen-Free Processed Product (1)

The present invention provides a method for producing a processed product of fish or fish egg in which an antigen is eliminated or reduced, the method comprising the step of confirming that the antigen is eliminated or reduced, in a production process of the processed product, wherein the antigen is at least one of the aforementioned antigens (10) to (14).

The step of confirming that the antigen is eliminated or reduced, in a production process of the processed product of fish or fish egg in which an antigen is eliminated or reduced may be performed by confirming the presence or absence of an antigen by the method described above in the subsection titled “Tester (1)”.

The production of the processed product offish or fish egg in which an antigen is eliminated or reduced may be performed by the method described above in the subsection titled “Allergen-free food and the like”.

### Epitope of Antigen

Epitopes and amino acids important for binding activity against IgE antibodies from allergic patients within the epitopes were identified as shown in Example 10 as to antigens identified as shown in Examples 2, 3 and 5 to 8 and known antigens aldolase and β-enolase of an allergy to salmon or the like and known antigen glyceraldehyde-3-phosphate dehydrogenase of an allergy to sardine.

The present invention provides polypeptides of the following (1α) to (17α) as polypeptides comprising an amino acid sequence specifically binding to an IgE antibody from an allergic patient.

### (1α) Epitope of Alpha-Actinin-3

In the present invention, the polypeptide (1α) can be any polypeptide selected from the group consisting of (1α-1) to (1α-6) as defined below:
(1α-1) a polypeptide comprising at least one amino acid sequence selected from the group consisting of SEQ ID NOs: 150-154, 207, 210, 213, 218, 224, and 227;
(1α-2) a polypeptide comprising the amino acid sequence of XXXXXXKPDX (SEQ ID NO: 205), preferably a polypeptide comprising the amino acid sequence of SXXXXXKPDK (SEQ ID NO: 206), more preferably a polypeptide comprising an amino acid sequence in which any 1, 2, 3, 4, 5, 6 or 7 of the amino acids at positions 1-6 and 10 of SEQ ID NO: 207 are substituted by other amino acids, preferably alanine, further preferably a polypeptide comprising an amino acid sequence in which any 1, 2, 3, 4 or 5 of the amino acids at positions 2-6 of SEQ ID NO: 207 are substituted by other amino acids, preferably alanine.

In another mode, a polypeptide comprising the amino acid sequence of DKXXXR (SEQ ID NO: 208), preferably a polypeptide comprising an amino acid sequence in which any 1, 2 or 3 of the amino acids at positions 3-5 of SEQ ID NO: 210 are substituted by other amino acids, preferably alanine.
(1α-3) a polypeptide comprising the amino acid sequence of XXXXXXXDPM (SEQ ID NO: 211), preferably a polypeptide comprising the amino acid sequence of YXXXXKDDPM (SEQ ID NO: 212), more preferably a polypeptide comprising an amino acid sequence in which any 1, 2, 3, 4, 5, 6 or 7 of the amino acids at positions 1-7 of SEQ ID NO: 213 are substituted by other amino acids, preferably alanine, further preferably a polypeptide comprising an amino acid sequence in which any 1, 2, 3 or 4 of the amino acids at positions 2-5 of SEQ ID NO: 213 are substituted by other amino acids, preferably alanine.

In another mode, a polypeptide comprising the amino acid sequence of XXXXXXDXPM (SEQ ID NO: 214), preferably a polypeptide comprising the amino acid sequence of YSXXXXDXPM (SEQ ID NO: 215), more preferably a polypeptide comprising an amino acid sequence in which any 1, 2, 3, 4, 5, 6 or 7 of the amino acids at positions 1-6 and 8 of SEQ ID NO: 213 are substituted by other amino acids, preferably alanine, further preferably a polypeptide comprising an amino acid sequence in which any 1, 2, 3, 4 or 5 of the amino acids at positions 3-6 and 8 of SEQ ID NO: 213 are substituted by other amino acids, preferably alanine.

In yet another mode, a polypeptide comprising the amino acid sequence of PXGXLXX (SEQ ID NO: 216), preferably a polypeptide comprising the amino acid sequence of PXGNLNT (SEQ ID NO: 217), more preferably a polypeptide comprising an amino acid sequence in which any 1, 2, 3 or 4 of the amino acids at positions 2, 4, 6 and 7 of SEQ ID NO: 218 are substituted by other amino acids, preferably alanine, further preferably a polypeptide comprising an amino acid sequence in which the amino acid at position 2 of SEQ ID NO: 218 is substituted by another amino acid, preferably alanine.
(1α-4) a polypeptide comprising the amino acid sequence of SXFYHAFAGAEQAET (SEQ ID NO: 219), preferably a polypeptide comprising an amino acid sequence in which the amino acid at position 2 of SEQ ID NO: 152 is substituted by another amino acid, preferably alanine.
(1α-5) a polypeptide comprising the amino acid sequence of TXLXXXNXPXXXXSE (SEQ ID NO: 220), preferably a polypeptide comprising the amino acid sequence of TXLRLXNRPXXXXSE (SEQ ID NO: 221), more preferably a polypeptide comprising an amino acid sequence in which any 1, 2, 3, 4, 5, 6, 7, 8 or 9 of the amino acids at positions 2, 4-6, 8 and 10-13 of SEQ ID NO: 153 are substituted by other amino acids, preferably alanine, further preferably a polypeptide comprising an amino acid sequence in which any 1, 2, 3, 4, 5 or 6 of the amino acids at positions 2, 6 and 10-13 of SEQ ID NO: 153 are substituted by other amino acids, preferably alanine.
(1α-6) a polypeptide comprising the amino acid sequence of SXKTXXXXXEXR (SEQ ID NO: 222), preferably a polypeptide comprising the amino acid sequence of SDKTXXXXXELR (SEQ ID NO: 223), more preferably a polypeptide comprising an amino acid sequence in which any 1, 2, 3, 4, 5, 6 or 7 of the amino acids at positions 2, 5-9 and 11 of SEQ ID NO: 224 are substituted by other amino acids, preferably alanine, further preferably a polypeptide comprising an amino acid sequence in which any 1, 2, 3, 4 or 5 of the amino acids at positions 5-9 of SEQ ID NO: 224 are substituted by other amino acids, preferably alanine.

In another mode, a polypeptide comprising the amino acid sequence of XXRE (SEQ ID NO: 225), preferably a polypeptide comprising the amino acid sequence of LXRE (SEQ ID NO: 226), more preferably a polypeptide comprising an amino acid sequence in which any 1 or 2 of the amino acids at positions 1 and 2 of SEQ ID NO: 227 are substituted by other amino acids, preferably alanine, further preferably a polypeptide comprising an amino acid sequence in which the amino acid at position 2 of SEQ ID NO: 227 is substituted by another amino acid, preferably alanine.

In one mode, the polypeptide (1α) may not comprise a variant or a homolog having 100% or at least 90%, at least 80% or at least 70% identity to the full-length amino acid sequence (SEQ ID NO: 2) of alpha-actinin-3.

### (2α) Epitope of EEF1A2 Binding Protein-Like

In the present invention, the polypeptide (2α) can be any polypeptide selected from the group consisting of (2α-1) and (2α-2) as defined below:
(2α-1) a polypeptide comprising at least one amino acid sequence selected from the group consisting of SEQ ID NOs: 155 and 230.
(2α-2) a polypeptide comprising the amino acid sequence of XXNRXYYXXXE (SEQ ID NO: 228), preferably a polypeptide comprising the amino acid sequence of LDNRLYYXVAE (SEQ ID NO: 229), more preferably a polypeptide comprising an amino acid sequence in which any 1, 2, 3, 4, 5 or 6 of the amino acids at positions 1, 2, 5 and 8-10 of SEQ ID NO: 230 are substituted by other amino acids, preferably alanine, further preferably a polypeptide comprising an amino acid sequence in which the amino acid at position 8 of SEQ ID NO: 230 is substituted by another amino acid, preferably alanine.

In one mode, the polypeptide (2α) may not comprise a variant or a homolog having 100% or at least 90%, at least 80% or at least 70% identity to the full-length amino acid sequence (SEQ ID NO: 5) of EEF1A2 binding protein-like.

### (3α) Epitope of Alpha-1,4-Glucan Phosphorylase

In the present invention, the polypeptide (3α) can be any polypeptide selected from the group consisting of (3α-1) to (3α-3) as defined below:
(3α-1) a polypeptide comprising at least one amino acid sequence selected from the group consisting of SEQ ID NOs: 156, 157, 232, 235, and 237.
(3α-2) a polypeptide comprising the amino acid sequence of YXXXXXXR (SEQ ID NO: 231), preferably a polypeptide comprising an amino acid sequence in which any 1, 2, 3, 4, 5 or 6 of the amino acids at positions 2-7 of SEQ ID NO: 232 are substituted by other amino acids, preferably alanine.

In another mode, a polypeptide comprising the amino acid sequence of GGYXQXXLXR (SEQ ID NO: 233), preferably a polypeptide comprising the amino acid sequence of GGYIQXXLDR (SEQ ID NO: 234), more preferably a polypeptide comprising an amino acid sequence in which any 1, 2, 3 or 4 of the amino acids at positions 4, 6, 7 and 9 of SEQ ID NO: 235 are substituted by other amino acids, preferably alanine, further preferably a polypeptide comprising an amino acid sequence in which any 1 or 2 of the amino acids at positions 6 and 7 of SEQ ID NO: 235 are substituted by other amino acids, preferably alanine.
(3α-3) a polypeptide comprising the amino acid sequence of RXKXXXDY (SEQ ID NO: 236), preferably a polypeptide comprising an amino acid sequence in which any 1, 2, 3 or 4 of the amino acids at positions 2 and 4-6 of SEQ ID NO: 237 are substituted by other amino acids, preferably alanine.

In one mode, the polypeptide (3α) may not comprise a variant or a homolog having 100% or at least 90%, at least 80% or at least 70% identity to the full-length amino acid sequence (SEQ ID NO: 10) of alpha-1,4-glucan phosphorylase.

### (4α) Epitope of Elongation Factor 2

In the present invention, the polypeptide (4α) can be any polypeptide selected from the group consisting of (4α-1) to (4α-3) as defined below:
(4α-1) a polypeptide comprising at least one amino acid sequence selected from the group consisting of SEQ ID NOs: 158, 159, 239, 242, 245, and 247.
(4α-2) a polypeptide comprising the amino acid sequence of KKXXXR (SEQ ID NO: 238), preferably a polypeptide comprising an amino acid sequence in which any 1, 2 or 3 of the amino acids at positions 3-5 of SEQ ID NO: 239 are substituted by other amino acids, preferably alanine.

In another mode, a polypeptide comprising the amino acid sequence of KKXXXRN (SEQ ID NO: 240), preferably a polypeptide comprising the amino acid sequence of KKSNXRN (SEQ ID NO: 241), more preferably a polypeptide comprising an amino acid sequence in which any 1, 2 or 3 of the amino acids at positions 3-5 of SEQ ID NO: 242 are substituted by other amino acids, preferably alanine, further preferably a polypeptide comprising an amino acid sequence in which the amino acid at position 5 of SEQ ID NO: 242 is substituted by another amino acid, preferably alanine.
(4α-3) a polypeptide comprising the amino acid sequence of LXXXLXXKXXI (SEQ ID NO: 243), preferably a polypeptide comprising the amino acid sequence of LXDXLXXKXXI (SEQ ID NO: 244), more preferably a polypeptide comprising an amino acid sequence in which any 1, 2, 3, 4, 5, 6 or 7 of the amino acids at positions 2-4, 6, 7, 9 and 10 of SEQ ID NO: 245 are substituted by other amino acids, preferably alanine, further preferably a polypeptide comprising an amino acid sequence in which any 1, 2, 3, 4, 5 or 6 of the amino acids at positions 2, 4, 6, 7, 9 and 10 of SEQ ID NO: 245 are substituted by other amino acids, preferably alanine.

In another mode, a polypeptide comprising the amino acid sequence of GKXXXXXXXXS (SEQ ID NO: 246), preferably a polypeptide comprising an amino acid sequence in which any 1, 2, 3, 4, 5, 6, 7 or 8 of the amino acids at positions 3-10 of SEQ ID NO: 247 are substituted by other amino acids, preferably alanine.

In one mode, the polypeptide (4α) may not comprise a variant or a homolog having 100% or at least 90%, at least 80% or at least 70% identity to the full-length amino acid sequence (SEQ ID NO: 19) of elongation factor 2.

### (5α) Epitope of Heat Shock Cognate 70 kDa Protein

In the present invention, the polypeptide (5α) can be any polypeptide selected from the group consisting of (5α-1) to (5α-4) as defined below:
(5α-1) a polypeptide comprising at least one amino acid sequence selected from the group consisting of SEQ ID NOs: 160-162, 250, 252 (amino acid sequence: QYK), 255, 258, and 261.
(5α-2) a polypeptide comprising the amino acid sequence of EXXXXYL (SEQ ID NO: 248), preferably a polypeptide comprising the amino acid sequence of EIXXAYL (SEQ ID NO: 249), more preferably a polypeptide comprising an amino acid sequence in which any 1, 2, 3 or 4 of the amino acids at positions 2-5 of SEQ ID NO: 250 are substituted by other amino acids, preferably alanine, further preferably a polypeptide comprising an amino acid sequence in which any 1 or 2 of the amino acids at positions 3 and 4 of SEQ ID NO: 250 are substituted by other amino acids, preferably alanine.
(5α-3) a polypeptide comprising the amino acid sequence of QXK (SEQ ID NO: 251), preferably a polypeptide comprising an amino acid sequence in which the amino acid at position 2 of SEQ ID NO: 252 is substituted by another amino acid, preferably alanine.

In another mode, a polypeptide comprising the amino acid sequence of XDXXXDK (SEQ ID NO: 253), preferably a polypeptide comprising the amino acid sequence of DDVXXDK (SEQ ID NO: 254), more preferably a polypeptide comprising an amino acid sequence in which any 1, 2, 3 or 4 of the amino acids at positions 1 and 3-5 of SEQ ID NO: 255 are substituted by other amino acids, preferably alanine, further preferably a polypeptide comprising an amino acid sequence in which any 1 or 2 of the amino acids at positions 4 and 5 of SEQ ID NO: 255 are substituted by other amino acids, preferably alanine.
(5α-4) a polypeptide comprising the amino acid sequence of KXSXEXK (SEQ ID NO: 256), preferably a polypeptide comprising the amino acid sequence of KLSXEDK (SEQ ID NO: 257), more preferably a polypeptide comprising an amino acid sequence in which any 1, 2 or 3 of the amino acids at positions 2, 4 and 6 of SEQ ID NO: 258 are substituted by other amino acids, preferably alanine, further preferably a polypeptide comprising an amino acid sequence in which the amino acid at position 4 of SEQ ID NO: 258 is substituted by another amino acid, preferably alanine.

In another mode, a polypeptide comprising the amino acid sequence of QXXXDKC (SEQ ID NO: 259), preferably a polypeptide comprising the amino acid sequence of QKIXDKC (SEQ ID NO: 260), more preferably a polypeptide comprising an amino acid sequence in which any 1, 2 or 3 of the amino acids at positions 2-4 of SEQ ID NO: 261 are substituted by other amino acids, preferably alanine, further preferably a polypeptide comprising an amino acid sequence in which the amino acid at position 4 of SEQ ID NO: 261 is substituted by another amino acid, preferably alanine.

In one mode, the polypeptide (5α) may not comprise a variant or a homolog having 100% or at least 90%, at least 80% or at least 70% identity to the full-length amino acid sequence (SEQ ID NO: 26) of heat shock cognate 70 kDa protein.

### (6α) Epitope of Serotransferrin

In the present invention, the polypeptide (6α) can be any polypeptide selected from the group consisting of (6α-1) to (6α-5) as defined below:
(6α-1) a polypeptide comprising at least one amino acid sequence selected from the group consisting of SEQ ID NOs: 163-167, 263, 266, 269, 271, 273, 276, and 279.
(6α-2) a polypeptide comprising the amino acid sequence of XXCYY (SEQ ID NO: 262), preferably a polypeptide comprising an amino acid sequence in which any 1 or 2 of the amino acids at positions 1 and 2 of SEQ ID NO: 263 are substituted by other amino acids, preferably alanine.

In another mode, a polypeptide comprising the amino acid sequence of VXVXKKXXX (SEQ ID NO: 264), preferably a polypeptide comprising the amino acid sequence of VAVAKKGXE (SEQ ID NO: 265), more preferably a polypeptide comprising an amino acid sequence in which any 1, 2, 3, 4 or 5 of the amino acids at positions 2, 4 and 7-9 of SEQ ID NO: 266 are substituted by other amino acids, preferably alanine, further preferably a polypeptide comprising an amino acid sequence in which the amino acid at position 8 of SEQ ID NO: 266 is substituted by another amino acid, preferably alanine.
(6α-3) a polypeptide comprising the amino acid sequence of XKXXXXEX (SEQ ID NO: 267), preferably a polypeptide comprising the amino acid sequence of XKXGXGEX (SEQ ID NO: 268), more preferably a polypeptide comprising an amino acid sequence in which any 1, 2, 3, 4, 5 or 6 of the amino acids at positions 1, 3-6 and 8 of SEQ ID NO: 269 are substituted by other amino acids, preferably alanine, further preferably a polypeptide comprising an amino acid sequence in which any 1, 2, 3 or 4 of the amino acids at positions 1, 3, 5 and 8 of SEQ ID NO: 269 are substituted by other amino acids, preferably alanine.
(6α-4) a polypeptide comprising the amino acid sequence of XXKXM (SEQ ID NO: 270), preferably a polypeptide comprising an amino acid sequence in which any 1, 2 or 3 of the amino acids at positions 1, 2 and 4 of SEQ ID NO: 271 are substituted by other amino acids, preferably alanine.

In another mode, a polypeptide comprising the amino acid sequence of VTNFXXXS (SEQ ID NO: 272), preferably a polypeptide comprising an amino acid sequence in which any 1, 2 or 3 of the amino acids at positions 5-7 of SEQ ID NO: 273 are substituted by other amino acids, preferably alanine.
(6α-5) a polypeptide comprising the amino acid sequence of YXYXXXXXC (SEQ ID NO: 274), preferably a polypeptide comprising the amino acid sequence of YXYNXXFXC (SEQ ID NO: 275), more preferably a polypeptide comprising an amino acid sequence in which any 1, 2, 3, 4, 5 or 6 of the amino acids at positions 2 and 4-8 of SEQ ID NO: 276 are substituted by other amino acids, preferably alanine, further preferably a polypeptide comprising an amino acid sequence in which any 1, 2, 3 or 4 of the amino acids at positions 2, 5, 6 and 8 of SEQ ID NO: 276 are substituted by other amino acids, preferably alanine.

In another mode, a polypeptide comprising the amino acid sequence of XXCLV (SEQ ID NO: 277), preferably a polypeptide comprising the amino acid sequence of FXCLV (SEQ ID NO: 278), more preferably a polypeptide comprising an amino acid sequence in which any 1 or 2 of the amino acids at positions 1 and 2 of SEQ ID NO: 279 are substituted by other amino acids, preferably alanine, further preferably a polypeptide comprising an amino acid sequence in which the amino acid at position 2 of SEQ ID NO: 279 is substituted by another amino acid, preferably alanine.

In one mode, the polypeptide (6α) may not comprise a variant or a homolog having 100% or at least 90%, at least 80% or at least 70% identity to the full-length amino acid sequence (SEQ ID NO: 33) of serotransferrin.

### (7α) Epitope of Myosin Binding Protein H-Like

In the present invention, the polypeptide (7α) can be any polypeptide selected from the group consisting of (7α-1) to (7α-5) as defined below:
(7α-1) a polypeptide comprising at least one amino acid sequence selected from the group consisting of SEQ ID NOs: 168-171, 282, 284, 287, 290, 292, 295, 297 and 300.
(7α-2) a polypeptide comprising the amino acid sequence of YVKXXXXKI (SEQ ID NO: 280), preferably polypeptide comprising the amino acid sequence of YVKXVXEKI (SEQ ID NO: 281), more preferably a polypeptide comprising an amino acid sequence in which any 1, 2, 3 or 4 of the amino acids at positions 4-7 of SEQ ID NO: 282 are substituted by other amino acids, preferably alanine, further preferably a polypeptide comprising an amino acid sequence in which any 1 or 2 of the amino acids at positions 4 and 6 of SEQ ID NO: 282 are substituted by other amino acids, preferably alanine.

In another mode, a polypeptide comprising the amino acid sequence of NIXIP (SEQ ID NO: 283), preferably a polypeptide comprising an amino acid sequence in which the amino acid at position 3 of SEQ ID NO: 284 is substituted by another amino acid, preferably alanine.
(7α-3) a polypeptide comprising the amino acid sequence of SXEXXXKXXXF (SEQ ID NO: 285), preferably a polypeptide comprising the amino acid sequence of SXEXCXKXXXF (SEQ ID NO: 286), more preferably a polypeptide comprising an amino acid sequence in which any 1, 2, 3, 4, 5, 6 or 7 of the amino acids at positions 2, 4-6 and 8-10 of SEQ ID NO: 287 are substituted by other amino acids, preferably alanine, further preferably a polypeptide comprising an amino acid sequence in which any 1, 2, 3, 4, 5 or 6 of the amino acids at positions 2, 4, 6, and 8-10 of SEQ ID NO: 287 are substituted by other amino acids, preferably alanine.

In another mode, a polypeptide comprising the amino acid sequence of KXDXFXXK (SEQ ID NO: 288), preferably a polypeptide comprising the amino acid sequence of KXDXFXDK (SEQ ID NO: 289), more preferably a polypeptide comprising an amino acid sequence in which any 1, 2, 3 or 4 of the amino acids at positions 2, 4, 6 and 7 of SEQ ID NO: 290 are substituted by other amino acids, preferably alanine, further preferably a polypeptide comprising an amino acid sequence in which any 1, 2 or 3 of the amino acids at positions 2, 4 and 6 of SEQ ID NO: 290 are substituted by other amino acids, preferably alanine.
(7α-4) a polypeptide comprising the amino acid sequence of EXXXYXXXXXXXXD (SEQ ID NO: 291), preferably a polypeptide comprising an amino acid sequence in which any 1, 2, 3, 4, 5, 6, 7, 8, 9, 10 or 11 of the amino acids at positions 2-4 and 6-13 of SEQ ID NO: 292 are substituted by other amino acids, preferably alanine.
(7α-5) a polypeptide comprising the amino acid sequence of XNXXYXXIXX (SEQ ID NO: 293), preferably a polypeptide comprising the amino acid sequence of DNXXYXXIXT (SEQ ID NO: 294), more preferably a polypeptide comprising an amino acid sequence in which any 1, 2, 3, 4, 5, 6 or 7 of the amino acids at positions 1, 3, 4, 6, 7, 9 and 10 of SEQ ID NO: 295 are substituted by other amino acids, preferably alanine, further preferably a polypeptide comprising an amino acid sequence in which any 1, 2, 3, 4 or 5 of the amino acids at positions 3, 4, 6, 7 and 9 of SEQ ID NO: 295 are substituted by other amino acids, preferably alanine.

In another mode, a polypeptide comprising the amino acid sequence of YXXIXT (SEQ ID NO: 296), preferably a polypeptide comprising an amino acid sequence in which any 1, 2 or 3 of the amino acids at positions 2, 3 and 5 of SEQ ID NO: 297 are substituted by other amino acids, preferably alanine.

In another mode, a polypeptide comprising the amino acid sequence of XXXISXGG (SEQ ID NO: 298), preferably a polypeptide comprising the amino acid sequence of YXMISXGG (SEQ ID NO: 299), more preferably a polypeptide comprising an amino acid sequence in which any 1, 2, 3 or 4 of the amino acids at positions 1-3 and 6 of SEQ ID NO: 300 are substituted by other amino acids, preferably alanine, further preferably a polypeptide comprising an amino acid sequence in which any 1 or 2 of the amino acids at positions 2 and 6 of SEQ ID NO: 300 are substituted by other amino acids, preferably alanine.

In one mode, the polypeptide (7α) may not comprise a variant or a homolog having 100% or at least 90%, at least 80% or at least 70% identity to the full-length amino acid sequence (SEQ ID NO: 43) of myosin binding protein H-like.

### (8α) Epitope of Desmin (Fragment)

In the present invention, the polypeptide (8α) can be any polypeptide selected from the group consisting of (8α-1) to (8α-4) as defined below:
(8α-1) a polypeptide comprising at least one amino acid sequence selected from the group consisting of SEQ ID NOs: 172, 173, 174, 303, 306, 308, and 310.
(8α-2) a polypeptide comprising the amino acid sequence of KXXXSX (SEQ ID NO: 301), preferably a polypeptide comprising the amino acid sequence of KXXXSD (SEQ ID NO: 302), more preferably a polypeptide comprising an amino acid sequence in which any 1, 2, 3 or 4 of the amino acids at positions 2-4 and 6 of SEQ ID NO: 303 are substituted by other amino acids, preferably alanine, further preferably a polypeptide comprising an amino acid sequence in which any 1, 2 or 3 of the amino acids at positions 2-4 of SEQ ID NO: 303 are substituted by other amino acids, preferably alanine.

In another mode, a polypeptide comprising the amino acid sequence of XKXKXXXXN (SEQ ID NO: 304), preferably a polypeptide comprising the amino acid sequence of YKSKXSDLN (SEQ ID NO: 305), more preferably a polypeptide comprising an amino acid sequence in which any 1, 2, 3, 4, 5 or 6 of the amino acids at positions 1, 3 and 5-8 of SEQ ID NO: 306 are substituted by other amino acids, preferably alanine, further preferably a polypeptide comprising an amino acid sequence in which the amino acid at position 5 of SEQ ID NO: 306 is substituted by another amino acid, preferably alanine.

In yet another mode, a polypeptide comprising the amino acid sequence of VXKN (SEQ ID NO: 307), preferably a polypeptide comprising an amino acid sequence in which the amino acid at position 2 of SEQ ID NO: 308 is substituted by another amino acid, preferably alanine.
(8α-4) a polypeptide comprising the amino acid sequence of DXGRXXE (SEQ ID NO: 309), preferably a polypeptide comprising an amino acid sequence in which any 1, 2 or 3 of the amino acids at positions 2, 5 and 6 of SEQ ID NO: 310 are substituted by other amino acids, preferably alanine.

In one mode, the polypeptide (8α) may not comprise a variant or a homolog having 100% or at least 90%, at least 80% or at least 70% identity to the amino acid sequence (SEQ ID NO: 56) of desmin (fragment).

### (9α) Epitope of Capping Protein (Actin Filament) Muscle Z-Line Beta

In the present invention, the polypeptide (9α) can be any polypeptide selected from the group consisting of (9α-1) to (9α-5) as defined below:
(9α-1) a polypeptide comprising at least one amino acid sequence selected from the group consisting of SEQ ID NOs: 175-178, 312, 314, 317, 320, 323, and 326.
(9α-2) a polypeptide comprising the amino acid sequence of KKXXXG (SEQ ID NO: 311), preferably a polypeptide comprising an amino acid sequence in which any 1, 2 or 3 of the amino acids at positions 3-5 of SEQ ID NO: 312 are substituted by other amino acids, preferably alanine.

In another mode, a polypeptide comprising the amino acid sequence of SKXXK (SEQ ID NO: 313), preferably a polypeptide comprising an amino acid sequence in which any 1 or 2 of the amino acids at positions 3 and 4 of SEQ ID NO: 314 are substituted by other amino acids, preferably alanine.
(9α-3) a polypeptide comprising the amino acid sequence of XXXXXQXKXXG (SEQ ID NO: 315), preferably a polypeptide comprising the amino acid sequence of HXXXXQXKSSG (SEQ ID NO: 316), more preferably a polypeptide comprising an amino acid sequence in which any 1, 2, 3, 4, 5, 6, 7 or 8 of the amino acids at positions 1-5, 7, 9 and 10 of SEQ ID NO: 317 are substituted by other amino acids, preferably alanine, further preferably a polypeptide comprising an amino acid sequence in which any 1, 2, 3, 4 or 5 of the amino acids at positions 2-5 and 7 of SEQ ID NO: 317 are substituted by other amino acids, preferably alanine.
(9α-4) a polypeptide comprising the amino acid sequence of XYXGKXXXX (SEQ ID NO: 318), preferably a polypeptide comprising the amino acid sequence of IYXGKTXDI (SEQ ID NO: 319), more preferably a polypeptide comprising an amino acid sequence in which any 1, 2, 3, 4, 5 or 6 of the amino acids at positions 1, 3 and 6-9 of SEQ ID NO: 320 are substituted by other amino acids, preferably alanine, further preferably a polypeptide comprising an amino acid sequence in which any 1 or 2 of the amino acids at positions 3 and 7 of SEQ ID NO: 320 are substituted by other amino acids, preferably alanine.

In another mode, a polypeptide comprising the amino acid sequence of XDXXNXXRS (SEQ ID NO: 321), preferably a polypeptide comprising the amino acid sequence of KDXXNXLRS (SEQ ID NO: 322), more preferably a polypeptide comprising an amino acid sequence in which any 1, 2, 3, 4 or 5 of the amino acids at positions 1, 3, 4, 6 and 7 of SEQ ID NO: 323 are substituted by other amino acids, preferably alanine, further preferably a polypeptide comprising an amino acid sequence in which any 1, 2 or 3 of the amino acids at positions 3, 4 and 6 of SEQ ID NO: 323 are substituted by other amino acids, preferably alanine.
(9α-5) a polypeptide comprising the amino acid sequence of QKYRQXXKXXXX (SEQ ID NO: 324), preferably a polypeptide comprising the amino acid sequence of QKYRQXXKXLXQ (SEQ ID NO: 325), more preferably a polypeptide comprising an amino acid sequence in which any 1, 2, 3, 4, 5 or 6 of the amino acids at positions 6, 7 and 9-12 of SEQ ID NO: 326 are substituted by other amino acids, preferably alanine, further preferably a polypeptide comprising an amino acid sequence in which any 1, 2, 3 or 4 of the amino acids at positions 6, 7, 9 and 11 of SEQ ID NO: 326 are substituted by other amino acids, preferably alanine.

In one mode, the polypeptide (9α) may not comprise a variant or a homolog having 100% or at least 90%, at least 80% or at least 70% identity to the full-length amino acid sequence (SEQ ID NO: 61) of capping protein (actin filament) muscle Z-line beta.

### (10α) Epitope of Myosin Heavy Chain, Fast Skeletal Muscle-Like

In the present invention, the polypeptide (10α) can be any polypeptide selected from the group consisting of (10α-1) to (10α-8) as defined below:
(10α-1) a polypeptide comprising at least one amino acid sequence selected from the group consisting of SEQ ID NOs: 179-185, 329, 332, 335, 338, 341, 344, 347, 350, 353, 356, 359, 362, and 365.
(10α-2) a polypeptide comprising the amino acid sequence of TXXXXDXXEGK (SEQ ID NO: 327), preferably a polypeptide comprising the amino acid sequence of TXXXLDFREGK (SEQ ID NO: 328), more preferably a polypeptide comprising an amino acid sequence in which any 1, 2, 3, 4, 5 or 6 of the amino acids at positions 2-5, 7 and 8 of SEQ ID NO: 329 are substituted by other amino acids, preferably alanine, further preferably a polypeptide comprising an amino acid sequence in which any 1, 2 or 3 of the amino acids at positions 2-4 of SEQ ID NO: 329 are substituted by other amino acids, preferably alanine.

In another mode, a polypeptide comprising the amino acid sequence of XXEXXEXXD (SEQ ID NO: 330), preferably a polypeptide comprising the amino acid sequence of FREXXEXXD (SEQ ID NO: 331), more preferably a polypeptide comprising an amino acid sequence in which any 1, 2, 3, 4, 5 or 6 of the amino acids at positions 1, 2, 4, 5, 7 and 8 of SEQ ID NO: 332 are substituted by other amino acids, preferably alanine, further preferably a polypeptide comprising an amino acid sequence in which any 1, 2, 3 or 4 of the amino acids at positions 4, 5, 7 and 8 of SEQ ID NO: 332 are substituted by other amino acids, preferably alanine.
(10α-3) a polypeptide comprising the amino acid sequence of LXXNFTXXK (SEQ ID NO: 333), preferably a polypeptide comprising the amino acid sequence of LXINFTNEK (SEQ ID NO: 334), more preferably a polypeptide comprising an amino acid sequence in which any 1, 2, 3 or 4 of the amino acids at positions 2, 3, 7 and 8 of SEQ ID NO: 335 are substituted by other amino acids, preferably alanine, further preferably a polypeptide comprising an amino acid sequence in which the amino acid at position 2 of SEQ ID NO: 335 is substituted by another amino acid, preferably alanine.
(10α-4) a polypeptide comprising the amino acid sequence of XYXPPPXXXK (SEQ ID NO: 336), preferably a polypeptide comprising the amino acid sequence of LYXPPPXXXK (SEQ ID NO: 337), more preferably a polypeptide comprising an amino acid sequence in which any 1, 2, 3, 4 or 5 of the amino acids at positions 1, 3 and 7-9 of SEQ ID NO: 338 are substituted by other amino acids, preferably alanine, further preferably a polypeptide comprising an amino acid sequence in which any 1, 2, 3 or 4 of the amino acids at positions 3 and 7-9 of SEQ ID NO: 338 are substituted by other amino acids, preferably alanine.
(10α-5) a polypeptide comprising the amino acid sequence of XXDEXVXK (SEQ ID NO: 339), preferably a polypeptide comprising the amino acid sequence of SXDEXVXK (SEQ ID NO: 340), more preferably a polypeptide comprising an amino acid sequence in which any 1, 2, 3 or 4 of the amino acids at positions 1, 2, 5 and 7 of SEQ ID NO: 341 are substituted by other amino acids, preferably alanine, further preferably a polypeptide comprising an amino acid sequence in which any 1, 2 or 3 of the amino acids at positions 2, 5 and 7 of SEQ ID NO: 341 are substituted by other amino acids, preferably alanine.

In another mode, a polypeptide comprising the amino acid sequence of XXDXXXAKXT (SEQ ID NO: 342), preferably a polypeptide comprising the amino acid sequence of SXDEXVAKXT (SEQ ID NO: 343), more preferably a polypeptide comprising an amino acid sequence in which any 1, 2, 3, 4, 5 or 6 of the amino acids at positions 1, 2, 4-6 and 9 of SEQ ID NO: 344 are substituted by other amino acids, preferably alanine, further preferably a polypeptide comprising an amino acid sequence in which any 1, 2 or 3 of the amino acids at positions 2, 5 and 9 of SEQ ID NO: 344 are substituted by other amino acids, preferably alanine.

In yet another mode, a polypeptide comprising the amino acid sequence of VXXXXKXKK (SEQ ID NO: 345), preferably a polypeptide comprising the amino acid sequence of VAXXXKXKK (SEQ ID NO: 346), more preferably a polypeptide comprising an amino acid sequence in which any 1, 2, 3, 4 or 5 of the amino acids at positions 2-5 and 7 of SEQ ID NO: 347 are substituted by other amino acids, preferably alanine, further preferably a polypeptide comprising an amino acid sequence in which any 1, 2, 3 or 4 of the amino acids at positions 3-5 and 7 of SEQ ID NO: 347 are substituted by other amino acids, preferably alanine.

In yet another mode, a polypeptide comprising the amino acid sequence of KXXKXK (SEQ ID NO: 348), preferably a polypeptide comprising the amino acid sequence of KXXKEK (SEQ ID NO: 349), more preferably a polypeptide comprising an amino acid sequence in which any 1, 2 or 3 of the amino acids at positions 2, 3 and 5 of SEQ ID NO: 350 are substituted by other amino acids, preferably alanine, further preferably a polypeptide comprising an amino acid sequence in which any 1 or 2 of the amino acids at positions 2 and 3 of SEQ ID NO: 350 are substituted by other amino acids, preferably alanine.
(10α-6) a polypeptide comprising the amino acid sequence of XNXXXKXKXK (SEQ ID NO: 351), preferably a polypeptide comprising the amino acid sequence of XNXXXKXKTK (SEQ ID NO: 352), more preferably a polypeptide comprising an amino acid sequence in which any 1, 2, 3, 4, 5 or 6 of the amino acids at positions 1, 3-5, 7 and 9 of SEQ ID NO: 353 are substituted by other amino acids, preferably alanine, further preferably a polypeptide comprising an amino acid sequence in which any 1, 2, 3, 4 or 5 of the amino acids at positions 1, 3-5 and 7 of SEQ ID NO: 353 are substituted by other amino acids, preferably alanine.
(10α-7) a polypeptide comprising the amino acid sequence of DXXXSXXK (SEQ ID NO: 354), preferably a polypeptide comprising the amino acid sequence of DXXXSXRK (SEQ ID NO: 355), more preferably a polypeptide comprising an amino acid sequence in which any 1, 2, 3, 4 or 5 of the amino acids at positions 2-4, 6 and 7 of SEQ ID NO: 356 are substituted by other amino acids, preferably alanine, further preferably a polypeptide comprising an amino acid sequence in which any 1, 2, 3 or 4 of the amino acids at positions 2-4 and 6 of SEQ ID NO: 356 are substituted by other amino acids, preferably alanine.

In another mode, a polypeptide comprising the amino acid sequence of SXXKXEX (SEQ ID NO: 357), preferably a polypeptide comprising the amino acid sequence of SXRKXEG (SEQ ID NO: 358), more preferably a polypeptide comprising an amino acid sequence in which any 1, 2, 3 or 4 of the amino acids at positions 2, 3, 5 and 7 of SEQ ID NO: 359 are substituted by other amino acids, preferably alanine, further preferably a polypeptide comprising an amino acid sequence in which any 1 or 2 of the amino acids at positions 2 and 5 of SEQ ID NO: 359 are substituted by other amino acids, preferably alanine.
(10α-8) a polypeptide comprising the amino acid sequence of XXRXXEXK (SEQ ID NO: 360), preferably a polypeptide comprising the amino acid sequence of EXRXXEEK (SEQ ID NO: 361), more preferably a polypeptide comprising an amino acid sequence in which any 1, 2, 3, 4 or 5 of the amino acids at positions 1, 2, 4, 5 and 7 of SEQ ID NO: 362 are substituted by other amino acids, preferably alanine, further preferably a polypeptide comprising an amino acid sequence in which any 1, 2 or 3 of the amino acids at positions 2, 4 and 5 of SEQ ID NO: 362 are substituted by other amino acids, preferably alanine.

In another mode, a polypeptide comprising the amino acid sequence of XXXEXKXKK (SEQ ID NO: 363), preferably a polypeptide comprising the amino acid sequence of RXXEEKXKK (SEQ ID NO: 364), more preferably a polypeptide comprising an amino acid sequence in which any 1, 2, 3, 4 or 5 of the amino acids at positions 1-3, 5 and 7 of SEQ ID NO: 365 are substituted by other amino acids, preferably alanine, further preferably a polypeptide comprising an amino acid sequence in which any 1, 2 or 3 of the amino acids at positions 2, 3 and 7 of SEQ ID NO: 365 are substituted by other amino acids, preferably alanine.

### (11α) Epitope of Glycogen Phosphorylase, Muscle Form-Like

In the present invention, the polypeptide (11α) can be any polypeptide selected from the group consisting of (11α-1) and (11α-2) as defined below:
(11α-1) a polypeptide comprising at least one amino acid sequence selected from the group consisting of SEQ ID NOs: 186, 367, and 370.
(11α-2) a polypeptide comprising the amino acid sequence of GXYXXXXXXR (SEQ ID NO: 366), preferably a polypeptide comprising an amino acid sequence in which any 1, 2, 3, 4, 5, 6 or 7 of the amino acids at positions 2 and 4-9 of SEQ ID NO: 367 are substituted by other amino acids, preferably alanine.

In another mode, a polypeptide comprising the amino acid sequence of NLXXN (SEQ ID NO: 368), preferably a polypeptide comprising the amino acid sequence of NLXEN (SEQ ID NO: 369), more preferably a polypeptide comprising an amino acid sequence in which any 1 or 2 of the amino acids at positions 3 and 4 of SEQ ID NO: 370 are substituted by other amino acids, preferably alanine, further preferably a polypeptide comprising an amino acid sequence in which the amino acid at position 3 of SEQ ID NO: 370 is substituted by another amino acid, preferably alanine.

### (12α) Epitope of Myosin-Binding Protein C, Fast-Type-Like

In the present invention, the polypeptide (12α) can be any polypeptide selected from the group consisting of (12α-1) to (12α-11) as defined below:
(12α-1) a polypeptide comprising at least one amino acid sequence selected from the group consisting of SEQ ID NOs: 187-196, 373, 376, 379, 382, 384, 386, 388, 390, 392, 395, 397, 400, 402, 405, 407, 409, 411, and 413.
(12α-2) a polypeptide comprising the amino acid sequence of KXTXXKKKXX (SEQ ID NO: 371), preferably a polypeptide comprising the amino acid sequence of KXTXXKKKPV (SEQ ID NO: 372), more preferably a polypeptide comprising an amino acid sequence in which any 1, 2, 3, 4 or 5 of the amino acids at positions 2, 4, 5, 9 and 10 of SEQ ID NO: 373 are substituted by other amino acids, preferably alanine, further preferably a polypeptide comprising an amino acid sequence in which any 1, 2 or 3 of the amino acids at positions 2, 4 and 5 of SEQ ID NO: 373 are substituted by other amino acids, preferably alanine.

In another mode, a polypeptide comprising the amino acid sequence of KXKPVVXX (SEQ ID NO: 374), preferably a polypeptide comprising the amino acid sequence of KXKPVVDE (SEQ ID NO: 375), more preferably a polypeptide comprising an amino acid sequence in which any 1, 2 or 3 of the amino acids at positions 2, 7 and 8 of SEQ ID NO: 376 are substituted by other amino acids, preferably alanine, further preferably a polypeptide comprising an amino acid sequence in which the amino acid at position 2 of SEQ ID NO: 376 is substituted by another amino acid, preferably alanine.
(12α-3) a polypeptide comprising the amino acid sequence of YXXXXFXXXI (SEQ ID NO: 377), preferably a polypeptide comprising the amino acid sequence of YXXIXFEYXI (SEQ ID NO: 378), more preferably a polypeptide comprising an amino acid sequence in which any 1, 2, 3, 4, 5, 6 or 7 of the amino acids at positions 2-5 and 7-9 of SEQ ID NO: 379 are substituted by other amino acids, preferably alanine, further preferably a polypeptide comprising an amino acid sequence in which any 1, 2, 3 or 4 of the amino acids at positions 2, 3, 5 and 9 of SEQ ID NO: 379 are substituted by other amino acids, preferably alanine.

In another mode, a polypeptide comprising the amino acid sequence of XXIXDXRG (SEQ ID NO: 380), preferably a polypeptide comprising the amino acid sequence of YXIXDXRG (SEQ ID NO: 381), more preferably a polypeptide comprising an amino acid sequence in which any 1, 2, 3 or 4 of the amino acids at positions 1, 2, 4 and 6 of SEQ ID NO: 382 are substituted by other amino acids, preferably alanine, further preferably a polypeptide comprising an amino acid sequence in which any 1, 2 or 3 of the amino acids at positions 2, 4 and 6 of SEQ ID NO: 382 are substituted by other amino acids, preferably alanine.
(12α-4) a polypeptide comprising the amino acid sequence of DXRGXXK (SEQ ID NO: 383), preferably a polypeptide comprising an amino acid sequence in which any 1, 2 or 3 of the amino acids at positions 2, 5 and 6 of SEQ ID NO: 384 are substituted by other amino acids, preferably alanine.
(12α-5) a polypeptide comprising the amino acid sequence of KMXXXXPK (SEQ ID NO: 385), preferably a polypeptide comprising an amino acid sequence in which any 1, 2, 3 or 4 of the amino acids at positions 3-6 of SEQ ID NO: 386 are substituted by other amino acids, preferably alanine.

In another mode, a polypeptide comprising the amino acid sequence of KXXKXXXPKH (SEQ ID NO: 387), preferably a polypeptide comprising an amino acid sequence in which any 1, 2, 3, 4 or 5 of the amino acids at positions 2, 3 and 5-7 of SEQ ID NO: 388 are substituted by other amino acids, preferably alanine.

In yet another mode, a polypeptide comprising the amino acid sequence of KXXXXFL (SEQ ID NO: 389), preferably a polypeptide comprising an amino acid sequence in which any 1, 2, 3 or 4 of the amino acids at positions 2-5 of SEQ ID NO: 390 are substituted by other amino acids, preferably alanine.
(12α-6) a polypeptide comprising the amino acid sequence of KGKKXX (SEQ ID NO: 391), preferably a polypeptide comprising an amino acid sequence in which any 1 or 2 of the amino acids at positions 5 and 6 of SEQ ID NO: 392 are substituted by other amino acids, preferably alanine.

In another mode, a polypeptide comprising the amino acid sequence of KXKXXXLXXE (SEQ ID NO: 393), preferably a polypeptide comprising the amino acid sequence of KXKXXXLQXE (SEQ ID NO: 394), more preferably a polypeptide comprising an amino acid sequence in which any 1, 2, 3, 4, 5 or 6 of the amino acids at positions 2, 4-6, 8 and 9 of SEQ ID NO: 395 are substituted by other amino acids, preferably alanine, further preferably a polypeptide comprising an amino acid sequence in which any 1, 2, 3, 4 or 5 of the amino acids at positions 2, 4-6 and 9 of SEQ ID NO: 395 are substituted by other amino acids, preferably alanine.
(12α-7) a polypeptide comprising the amino acid sequence of TXKX (SEQ ID NO: 396), preferably a polypeptide comprising an amino acid sequence in which any 1 or 2 of the amino acids at positions 2 and 4 of SEQ ID NO: 397 are substituted by other amino acids, preferably alanine.

In another mode, a polypeptide comprising the amino acid sequence of KYXXKKXXX (SEQ ID NO: 398), preferably a polypeptide comprising the amino acid sequence of KYXXKKDGL (SEQ ID NO: 399), more preferably a polypeptide comprising an amino acid sequence in which any 1, 2, 3, 4 or 5 of the amino acids at positions 3, 4 and 7-9 of SEQ ID NO: 400 are substituted by other amino acids, preferably alanine, further preferably a polypeptide comprising an amino acid sequence in which any 1 or 2 of the amino acids at positions 3 and 4 of SEQ ID NO: 400 are substituted by other amino acids, preferably alanine.
(12α-8) a polypeptide comprising the amino acid sequence of YXXXPDG (SEQ ID NO: 401), preferably a polypeptide comprising an amino acid sequence in which any 1, 2 or 3 of the amino acids at positions 2-4 of SEQ ID NO: 402 are substituted by other amino acids, preferably alanine.

In another mode, a polypeptide comprising the amino acid sequence of DGYXXSXSXK (SEQ ID NO: 403), preferably a polypeptide comprising the amino acid sequence of DGYXLSLSXK (SEQ ID NO: 404), more preferably a polypeptide comprising an amino acid sequence in which any 1, 2, 3 or 4 of the amino acids at positions 4, 5, 7 and 9 of SEQ ID NO: 405 are substituted by other amino acids, preferably alanine, further preferably a polypeptide comprising an amino acid sequence in which any 1 or 2 of the amino acids at positions 4 and 9 of SEQ ID NO: 405 are substituted by other amino acids, preferably alanine.
(12α-9) a polypeptide comprising the amino acid sequence of PVTXX (SEQ ID NO: 406), preferably a polypeptide comprising an amino acid sequence in which any 1 or 2 of the amino acids at positions 4 and 5 of SEQ ID NO: 407 are substituted by other amino acids, preferably alanine.
(12α-10) a polypeptide comprising the amino acid sequence of QXXXXXXXDKXN (SEQ ID NO: 408), preferably a polypeptide comprising an amino acid sequence in which any 1, 2, 3, 4, 5, 6, 7 or 8 of the amino acids at positions 2-8 and 11 of SEQ ID NO: 409 are substituted by other amino acids, preferably alanine.
(12α-11) a polypeptide comprising the amino acid sequence of YXXXXXDXKT (SEQ ID NO: 410), preferably a polypeptide comprising an amino acid sequence in which any 1, 2, 3, 4, 5 or 6 of the amino acids at positions 2-6 and 8 of SEQ ID NO: 411 are substituted by other amino acids, preferably alanine.

In another mode, a polypeptide comprising the amino acid sequence of YXXQXXXK (SEQ ID NO: 412), preferably a polypeptide comprising an amino acid sequence in which any 1, 2, 3, 4 or 5 of the amino acids at positions 2, 3 and 5-7 of SEQ ID NO: 413 are substituted by other amino acids, preferably alanine.

### (13α) Epitope of ATP Synthase Subunit Beta, Mitochondrial

In the present invention, the polypeptide (13α) can be any polypeptide selected from the group consisting of (13α-1) and (13α-2) as defined below:
(13α-1) a polypeptide comprising at least one amino acid sequence selected from the group consisting of SEQ ID NOs: 197, 415, and 417.
(13α-2) a polypeptide comprising the amino acid sequence of GXYS (SEQ ID NO: 414), preferably a polypeptide comprising an amino acid sequence in which the amino acid at position 2 of SEQ ID NO: 415 is substituted by another amino acid, preferably alanine.

In another mode, a polypeptide comprising the amino acid sequence of YXXXXG (SEQ ID NO: 416), preferably a polypeptide comprising an amino acid sequence in which any 1, 2, 3 or 4 of the amino acids at positions 2-5 of SEQ ID NO: 417 are substituted by other amino acids, preferably alanine.

### (14α) Epitope of L-Lactate Dehydrogenase A Chain-Like

In the present invention, the polypeptide (14α) can be any polypeptide selected from the group consisting of (14α-1) and (14α-2) as defined below:
(14α-1) a polypeptide comprising at least one amino acid sequence selected from the group consisting of SEQ ID NOs: 198 and 420.
(14α-2) a polypeptide comprising the amino acid sequence of XPVGSXSKX (SEQ ID NO: 418), preferably a polypeptide comprising the amino acid sequence of EPVGSXSKV (SEQ ID NO: 419), more preferably a polypeptide comprising an amino acid sequence in which any 1, 2 or 3 of the amino acids at positions 1, 6 and 9 of SEQ ID NO: 420 are substituted by other amino acids, preferably alanine, further preferably a polypeptide comprising an amino acid sequence in which the amino acid at position 6 of SEQ ID NO: 420 is substituted by another amino acid, preferably alanine.

### (15α) Epitope of Aldolase

In the present invention, the polypeptide (15α) can be any polypeptide selected from the group consisting of (15α-1) and (15α-2) as defined below:
(15α-1) a polypeptide comprising at least one amino acid sequence selected from the group consisting of SEQ ID NOs: 199, 423, and 425.
(15α-2) a polypeptide comprising the amino acid sequence of XYXRXXXXXXXK (SEQ ID NO: 421), preferably a polypeptide comprising the amino acid sequence of SYXRXLXXXAXK (SEQ ID NO: 422), more preferably a polypeptide comprising an amino acid sequence in which any 1, 2, 3, 4, 5, 6, 7, 8 or 9 of the amino acids at positions 1, 3 and 5-11 of SEQ ID NO: 423 are substituted by other amino acids, preferably alanine, further preferably a polypeptide comprising an amino acid sequence in which any 1, 2, 3, 4, 5 or 6 of the amino acids at positions 3, 5, 7-9 and 11 of SEQ ID NO: 423 are substituted by other amino acids, preferably alanine.

In another mode, a polypeptide comprising the amino acid sequence of SXXKXXG (SEQ ID NO: 424), preferably a polypeptide comprising an amino acid sequence in which any 1, 2, 3 or 4 of the amino acids at positions 2, 3, 5 and 6 of SEQ ID NO: 425 are substituted by other amino acids, preferably alanine.

In one mode, the polypeptide (15α) may not comprise a variant or a homolog having 100% or at least 90%, at least 80% or at least 70% identity to the full-length amino acid sequence (NCBI protein accession number: NP_001133180.1, SEQ ID NO: 445) of aldolase.

### (16α) Epitope of β-Enolase

In the present invention, the polypeptide (16α) can be any polypeptide selected from the group consisting of (16α-1) to (16α-4) as defined below:
(16α-1) a polypeptide comprising at least one amino acid sequence selected from the group consisting of SEQ ID NOs: 200-202, 428, 431, 434, and 436.
(16α-2) a polypeptide comprising the amino acid sequence of RYLGKXXX (SEQ ID NO: 426), preferably a polypeptide comprising the amino acid sequence of RYLGKGXV (SEQ ID NO: 427), more preferably a polypeptide comprising an amino acid sequence in which any 1, 2 or 3 of the amino acids at positions 6-8 of SEQ ID NO: 428 are substituted by other amino acids, preferably alanine, further preferably a polypeptide comprising an amino acid sequence in which the amino acid at position 7 of SEQ ID NO: 428 is substituted by another amino acid, preferably alanine.
(16α-3) a polypeptide comprising the amino acid sequence of YXXIXDXXXH (SEQ ID NO: 429), preferably a polypeptide comprising the amino acid sequence of YXXIXDLXGH (SEQ ID NO: 430), more preferably a polypeptide comprising an amino acid sequence in which any 1, 2, 3, 4, 5 or 6 of the amino acids at positions 2, 3, 5 and 7-9 of SEQ ID NO: 431 are substituted by other amino acids, preferably alanine, further preferably a polypeptide comprising an amino acid sequence in which any 1, 2, 3 or 4 of the amino acids at positions 2, 3, 5 and 8 of SEQ ID NO: 431 are substituted by other amino acids, preferably alanine.

In another mode, a polypeptide comprising the amino acid sequence of DXXXHXDVXL (SEQ ID NO: 432), preferably a polypeptide comprising the amino acid sequence of DXXXHKDVIL (SEQ ID NO: 433), more preferably a polypeptide comprising an amino acid sequence in which any 1, 2, 3, 4 or 5 of the amino acids at positions 2-4, 6 and 9 of SEQ ID NO: 434 are substituted by other amino acids, preferably alanine, further preferably a polypeptide comprising an amino acid sequence in which any 1, 2 or 3 of the amino acids at positions 2-4 of SEQ ID NO: 434 are substituted by other amino acids, preferably alanine.
(16α-4) a polypeptide comprising the amino acid sequence of IKXXXXKDAT (SEQ ID NO: 435), preferably a polypeptide comprising an amino acid sequence in which any 1, 2, 3 or 4 of the amino acids at positions 3-6 of SEQ ID NO: 436 are substituted by other amino acids, preferably alanine.

In one mode, the polypeptide (16α) may not comprise a variant or a homolog having 100% or at least 90%, at least 80% or at least 70% identity to the full-length amino acid sequence (NCBI protein accession number: NP_001133193.1, SEQ ID NO: 446) of β-enolase.

### (17α) Epitope of Glyceraldehyde-3-Phosphate Dehydrogenase

In the present invention, the polypeptide (17α) can be any polypeptide selected from the group consisting of (17α-1) to (17α-3) as defined below:
(17α-1) a polypeptide comprising at least one amino acid sequence selected from the group consisting of SEQ ID NOs: 203, 204, 441, and 444.
(17α-2) a polypeptide comprising the amino acid sequence of YXXXXXXXDXXXGRF (SEQ ID NO: 437), preferably a polypeptide comprising the amino acid sequence of YXXXXXXXDSTXGRF (SEQ ID NO: 438), more preferably a polypeptide comprising an amino acid sequence in which any 1, 2, 3, 4, 5, 6, 7, 8, 9 or 10 of the amino acids at positions 2-8 and 10-12 of SEQ ID NO: 203 are substituted by other amino acids, preferably alanine, further preferably a polypeptide comprising an amino acid sequence in which any 1, 2, 3, 4, 5, 6, 7 or 8 of the amino acids at positions 2-8 and 12 of SEQ ID NO: 203 are substituted by other amino acids, preferably alanine.
(17α-3) a polypeptide comprising the amino acid sequence of SYXXIXXV (SEQ ID NO: 440), preferably a polypeptide comprising an amino acid sequence in which any 1, 2, 3 or 4 of the amino acids at positions 3, 4, 6 and 7 of SEQ ID NO: 441 are substituted by other amino acids, preferably alanine.

In another mode, a polypeptide comprising the amino acid sequence of IKKXXK (SEQ ID NO: 442), preferably a polypeptide comprising the amino acid sequence of IKKVXK (SEQ ID NO: 443), more preferably a polypeptide comprising an amino acid sequence in which any 1 or 2 of the amino acids at positions 4 and 5 of SEQ ID NO: 444 are substituted by other amino acids, preferably alanine, further preferably a polypeptide comprising an amino acid sequence in which the amino acid at position 5 of SEQ ID NO: 444 is substituted by another amino acid, preferably alanine.

In one mode, the polypeptide (17α) may not comprise a variant or a homolog having 100% or at least 90%, at least 80% or at least 70% identity to the full-length amino acid sequence (NCBI protein accession number: NP_001117033.1, SEQ ID NO: 447) of glyceraldehyde-3-phosphate dehydrogenase.

The lengths of the aforementioned polypeptides (1α) to (17α) are not particularly limited. In a preferred mode, the lengths of the aforementioned polypeptides (1α) to (17α) can be 500 amino acids or less, 300 amino acids or less, 200 amino acids or less, 100 amino acids or less, 50 amino acids or less, 30 amino acids or less, 20 amino acids or less, 15 amino acids or less, 10 amino acids or less, or 5 amino acids or less.

The aforementioned polypeptides (1α) to (17α) may be prepared by a technique of chemical synthesis such as solid-phase peptide synthesis. Alternatively, polypeptides comprising an epitope may be obtained by expressing them as recombinant proteins using a genetic recombination technique well known to those skilled in the art and by separating and producing them using protein producing methods well known to those skilled in the art.

### Diagnosis Kit and Method (2)

The present invention provides a method for providing an indicator for diagnosing an allergy in a subject, the method comprising the steps of:
(i) contacting a sample obtained from the subject with an antigen, wherein the sample is a solution comprising an IgE antibody;
(ii) detecting binding between the IgE antibody present in the sample obtained from the subject and the antigen; and
(iii) when the binding between the IgE antibody in the subject and the antigen is detected, an indicator of the fact that the subject is allergic is provided;
   wherein the antigen is a polypeptide that is at least one of the aforementioned polypeptides (1α) to (17α), or a polypeptide in which two or more of the aforementioned polypeptides (1α) to (17α) are joined together via or without a spacer.

Hereinafter, the polypeptide that is at least one of the aforementioned polypeptides (1α) to (17α), or the polypeptide in which two or more of the aforementioned polypeptides (1α) to (17α) are joined together via or without a spacer is referred to as the “antigen including (1α) to (17α)” in the present specification. The type of the spacer is not particularly limited, and an ordinary spacer that is used by those skilled in the art for joining together two or more peptides can be used. The spacer may be, for example, a hydrocarbon chain such as Acp(6)-OH.

The sample obtained from a subject is as described above in the subsection titled “Diagnosis kit and method (1)”.

Detection of contact and binding between the sample obtained from a subject and the antigen can be carried out by using a known method described above in the subsection titled “Diagnosis kit and method (1)”, such as ELISA (Enzyme-Linked Immunosorbent Assay), sandwich immunoassay, immunoblotting, immunoprecipitation, or immunochromatography.

The antigen including (1α) to (17α) may be provided in a state immobilized on a carrier. In this case, the steps (i) and (ii) mentioned above can be carried out using ELISA, sandwich immunoassay, immunochromatography, surface plasmon resonance, or the like. The step (i) mentioned above can be carried out by contacting the sample obtained from a subject with a surface on which the antigen including (1α) to (17α) is immobilized. The IgE antibody from the subject may be used in a state immobilized on a carrier, and binding to the antigen including (1α) to (17α) may be detected by the aforementioned technique.

The antigen including (1α) to (17α) may be in a state unimmobilized on a carrier. In this case, flow cytometry or the like can be used in the aforementioned steps (i) and (ii), and the presence of IgE antibody-bound antigen including (1α) to (17α) can be confirmed with laser beam. Examples of this method include a basophil activation test (BAT) which is a method in which a surface antigen CD203c that appears when basophils are activated by the contact of the antigen including (1α) to (17α) is detected. Another example includes a histamine release test (HRT) which examines whether histamine is released by further contacting the antigen including (1α) to (17α) with blood cells in a sample.

The antigen including (1α) to (17α) is an antigen specifically binding to IgE antibodies from allergic patients. Therefore, when binding between an IgE antibody from a subject and the antigen is detected, an indicator of the fact that the subject is allergic to fish is provided.

The present invention further provides a kit for diagnosing an allergy, comprising at least one antigen including (1α) to (17α). The diagnosis kit of this invention may be used in the aforementioned method for providing an indicator for diagnosing an allergy or in a diagnosis method as described later. The diagnosis kit of this invention may comprise not only the at least one antigen including (1α) to (17α), but also an anti-IgE antibody labeled with an enzyme and a chromogenic or luminescent substrate serving as a substrate for the enzyme. Also, a fluorescent-labeled anti-IgE antibody may be used. In the diagnosis kit of this invention, the antigen including (1α) to (17α) may be provided in a state immobilized on a carrier. The diagnosis kit of this invention may also be provided together with instructions on the procedure for diagnosis or a package containing said instructions.

In another mode, the aforementioned diagnosis kit comprises a companion diagnostic agent for an allergy. The companion diagnostic agent is used for identifying patients expected to respond to pharmaceutical products or identifying patients having the risk of severe adverse reactions to pharmaceutical products, or for studying the reactivity of pharmaceutical products in order to optimize treatment using the pharmaceutical products. Here, the optimization of treatment includes, for example, determination of dosage and administration, judgment regarding discontinuation of administration, and confirmation of an allergen component that is used to cause immunological tolerance.

The present invention further provides a composition for diagnosing an allergy, comprising at least one antigen including (1α) to (17α). The diagnosis composition of this invention can be used in a diagnosis method as described below. The diagnosis composition of this invention may further comprise a pharmaceutically acceptable carrier and/or additives commonly used with the antigen of this invention depending on the need.

In one mode, the present invention provides a method for diagnosing an allergy in a subject, the method comprising:
(i) contacting a sample obtained from the subject with an antigen;
(ii) detecting binding between an IgE antibody present in the sample obtained from the subject and the antigen; and
(iii) when the binding between the IgE antibody in the subject and the antigen is detected, diagnosing the subject as being allergic;
wherein the antigen is at least one of the proteins defined as the antigen including (1α) to (17α). In this method, the steps (i) and (ii) are performed as described above regarding the corresponding steps of the method for providing an indicator for diagnosing an allergy.

In another mode, the present invention provides a method for diagnosing an allergy in a subject, the method comprising administering to the subject at least one antigen including (1α) to (17α). This method may be performed in the form of a skin test characterized by applying the antigen including (1α) to (17α) onto the skin. Examples of the skin test include various forms of tests, such as: a prick test in which a diagnosis composition is applied onto the skin and then a tiny prick to such an extent as not to provoke bleeding is made in the skin to allow the antigen including (1α) to (17α) to penetrate the skin, thereby observing a skin reaction; a scratch test in which a diagnosis composition is applied onto the skin and then the skin is lightly scratched to observe a reaction; a patch test in which a diagnosis composition in the form of cream, ointment, etc. is applied onto the skin to observe a reaction; and an intracutaneous test in which the antigen including (1α) to (17α) is administered intracutaneously to observe a reaction. If a skin reaction such as swelling occurs in a skin portion to which the antigen including (1α) to (17α) has been applied, the subject of interest is diagnosed as having an allergy. The amount of the antigen including (1α) to (17α) to be applied to the skin in such tests can be, for example, not more than 100 μg per dose.

In the process of allergy diagnosis, a load test aiming to identify an antigen is often adopted. At least one antigen including (1α) to (17α) can be used as an active ingredient for a load test to diagnose an allergy. Here, the allergen component to be used in the load test may be a polypeptide that has been expressed and purified and may be a protein that has been expressed in food or cooking ingredients, such as rice-based vaccine expressing pollen allergens which are obtained by transforming rice with a gene of a cedar pollen antigen and expressing the antigen protein in the rice.

In still another mode, the present invention provides at least one antigen including (1α) to (17α), intended for use in the diagnosis of an allergy.

In still another mode, the present invention provides use of at least one antigen including (1α) to (17α) for the production of a diagnostic agent for an allergy.

In this subsection, the allergy to be diagnosed or detected can be allergies to the antigen including (1α) to (17α). Thus, detection of the allergy can be detection of not only an allergy to a single antigen including (1α) to (17α), but also allergies including cross-reactivity.

### Pharmaceutical Composition and Treatment Method (2)

The present invention provides a pharmaceutical composition comprising at least one antigen including (1α) to (17α). In one mode, the aforementioned pharmaceutical composition is used for the treatment of an allergy. The treatment of an allergy increases the limit amount of an antigen in which the allergy does not develop even if the antigen is incorporated into the body, and finally aims for the state where the allergy does not develop by the common amount of the antigen to be consumed (remission).

The present invention also provides a method for treating an allergy, the method comprising administering at least one antigen including (1α) to (17α) to a patient in need of a treatment for an allergy.

In another mode, the present invention provides at least one antigen including (1α) to (17α), intended for use in the treatment for an allergy. In yet another mode, the present invention provides use of at least one antigen including (1α) to (17α) for the production of a therapeutic agent for an allergy.

In the process of allergy treatment, a hyposensitization therapy aiming to induce immunological tolerance by administering an antigen to a patient is often adopted. The at least one antigen including (1α) to (17α) can be used as an active ingredient for hyposensitization therapy for an allergy. Here, the allergen component to be used in the hyposensitization therapy may be a polypeptide that has been expressed and purified and may be a polypeptide that has been expressed in food or a cooking ingredient, such as rice-based vaccine expressing pollen allergens.

The administration route, administration dose, frequency and/or period of the pharmaceutical composition of this invention, and other ingredients to be contained in the pharmaceutical composition, and the dosage form can be as described above in the subsection titled “Pharmaceutical composition and treatment method (1)”. In the case of using the antigen including (1α) to (17α), for example, the dose to an adult patient may be a dose of not more than 100 μg per dose.

In this subsection, the allergy to be treated can be allergies to the antigen including (1α) to (17α). Thus, treatment of the allergy can be treatment of not only an allergy to a single antigen including (1α) to (17α), but also allergies including cross-reactivity.

### Tester (2)

The present invention provides a tester comprising an antibody for at least one antigen including (1α) to (17α).

The antibody can be prepared by a conventional method. For example, the antibody may be prepared by immunizing a mammal such as rabbit with the aforementioned antigen including (1α) to (17α). The antibody may be an Ig antibody, a polyclonal antibody, a monoclonal antibody, or an antigen-binding fragment thereof (e.g., Fab, F(ab′)₂, Fab′).

Further, in the aforementioned tester, the antibody may be provided in a form bound to a carrier. The type of the carrier is not particularly limited as long as it is usable for detection of binding between an antibody and the antigen including (1α) to (17α). Any given carrier known to those skilled in the art can be used. Also, the antibody for the antigen including (1α) to (17α) is preferably an antibody for the epitopes described above in the subsection titled “Epitope of antigen”. This can attain a tester that can also detect cross-reactivity.

Examples of a method for determining the presence or absence of the antigen including (1α) to (17α) include the following methods:
a method in which a prepared tester comprising an antibody is contacted with a sample obtained from a raw material, a processed product, etc., ELISA or the like is used to detect whether there is a binding between the antibody and the antigen including (1α) to (17α) in the sample, and if the binding between the antibody and the antigen including (1α) to (17α) is detected, it is determined that the antigen remains in the raw material, the processed product, etc. of interest; and
a method in which filter paper is impregnated with a raw material, a processed product, etc. and reacted with an antibody solution so as to detect the antigen including (1α) to (17α) contained therein.

Another mode of the present invention includes a tester for determining the presence or absence of the antigen including (1α) to (17α) of an allergy in an object of interest, the tester comprising a primer appropriate for an epitope. The primer is not limited and may be designed so as to comprise, for example, a portion of the nucleotide sequence of a nucleic acid encoding any of the amino acid sequences defined above in (1α) to (17α), or a complementary strand thereof. Alternatively, the primer may be designed so as to be the nucleotide sequence of a region upstream of a portion encoding an epitope that is any of the amino acid sequences defined above in (1α) to (17α), in a nucleic acid encoding a protein comprising the epitope, or the nucleotide sequence of a complementary strand of a region downstream of the portion encoding the epitope. Examples of such a primer include a primer which is a portion of at least one of the nucleotide sequences of SEQ ID NOs: 1, 4, 9, 18, 25, 32, 42, 55, 60, 69, 109, 120, 137, 143, 448, 449 and 450 and/or a primer which is a portion of a sequence complementary to at least one of the nucleotide sequences of SEQ ID NOs: 1, 4, 9, 18, 25, 32, 42, 55, 60, 69, 109, 120, 137, 143, 448, 449 and 450. Here, the position of the epitope in the full-length sequence of an antigen is as defined in Table 2 of Example 10 given below. Particularly, when mRNA is of interest, the tester has a complementary primer of a poly-A tail.

For example, DNA is amplified by PCR (Polymerase Chain Reaction) including RT-PCR using templated DNA or mRNA obtained from a sample and the aforementioned primer, and the presence or absence of a nucleic acid encoding the amino acid sequences defined above in (1α) to (17α) in the sequence of the amplified DNA is determined to determine the presence or absence of the antigen including (1α) to (17α). Amplification methods by PCR for mRNA of interest can be exemplified by RACE. When one of amino acid sequences encoded by three possible open reading frames in the amplified DNA comprises any of the amino acid sequences defined above in (1α) to (17α), it is determined that the antigen is present. When no DNA is amplified, it is determined that the antigen is absent.

In one mode, the aforementioned tester is used to determine the presence or absence of the antigen including (1α) to (17α) in raw materials or processed products of interest in a food production line. The raw material may be a cooking ingredient or may be a cosmetic raw material, a pharmaceutical raw material or the like. The processed product may be an edible processed product or may be a cosmetic, a pharmaceutical product or the like. The tester may also be used for search for organism species contained in raw materials, may be used for quality inspection of production lines and pre-shipment products by manufacturers, or may be used for self-checking of the presence or absence of an antigen in raw materials or processed products of interest by consumers or users.

### Allergen-Free Raw Material and the Like

The present invention provides a raw material or a processed product in which at least one antigen including (1α) to (17α) is eliminated or reduced.

The method for eliminating or reducing the antigen of the present invention in a raw material or a processed product is not limited. The elimination or reduction of the antigen may be conducted by any method, as long as the method permits the elimination or reduction of the antigen including (1α) to (17α). For example, the techniques described above in the subsection titled “Allergen-free food and the like” may be used.

Elimination or reduction of at least one antigen including (1α) to (17α) may be achieved by eliminating or reducing the whole antigen or may be achieved by cleaving or removing the sequence moiety defined in (1α) to (17α) from the antigen protein. The “removal” includes deletion and modification of the whole or a portion of a sequence moiety defined above in (1α) to (17α).

For example, the raw material in which the antigen including (1α) to (17α) is eliminated or reduced may be obtained by preparing a raw material in which the expression of the antigen including (1α) to (17α) is knocked out, using a gene knock-out technique. Any technique known to those skilled in the art such as genetic modification can be used as the gene knock-out technique.

The processed product in which the antigen including (1α) to (17α) is eliminated or reduced may be a processed product of the raw material in which the antigen including (1α) to (17α) is eliminated or reduced, such as powdered milk obtained with purified peptide as a raw material. In the case of using an ordinary raw material, a treatment for eliminating or reducing the antigen including (1α) to (17α) is performed before or after preparation of a processed product. The techniques described in the subsection titled “Allergen-free food and the like” may be used as methods for eliminating or reducing the antigen including (1α) to (17α) in a processed product obtained with an ordinary raw material. Examples of the method for cleaving the antigen including (1α) to (17α) include a method in which the antigen is treated by cleavage with a particular digestive enzyme.

### Method for Producing Allergen-Free Processed Product (2)

The present invention provides a method for producing a processed product in which an antigen is eliminated or reduced, the method comprising the step of confirming that the antigen is eliminated or reduced, in a production process of the processed product, wherein the antigen is at least one of the aforementioned antigens including (1α) to (17α).

In the production method, elimination or reduction of an antigen means that at least one antigen including (1α) to (17α) is eliminated or reduced, or the sequence moiety defined in (1α) to (17α) are cleaved or removed from the antigen.

A technique of confirming that the antigen is eliminated or reduced in the production process of the processed product is not particularly limited, and any technique capable of detecting at least one of the aforementioned antigen including (1α) to (17α) may be used. For example, the presence or absence of the polypeptide or the antigen in the processed product may be confirmed from the binding activity of an antibody for at least one of the aforementioned antigen including (1α) to (17α) against a sample containing a material resulting from the production process of the processed product. Details of such a method are as described above in the subsection titled “Diagnosis kit and method (2)”. Thus, in the production method, the “IgE antibody from a subject” described above in the subsection titled “Diagnosis kit and method (2)” is replaced with the “antibody for at least one of the aforementioned antigen including (1α) to (17α)”, and the “antigen” described above in the subsection titled “Diagnosis kit and method (2)” is replaced with the “sample containing a material resulting from the production process of the processed product”. The techniques described above in the subsection titled “Diagnosis kit and method (2)” can be used to confirm that the antigen is eliminated or reduced in the production process of the processed product. The testers described above in the subsection titled “Tester (2)” can also be used.

### EXAMPLES

The following describes examples of the present invention. The technical scope of this invention is not limited by these examples.

### Example 1: Confirmation of a Protein Pattern

Proteins contained in salmons were investigated using a two-dimensional electrophoresis method described below.

### Protein Extraction

Extraction and purification of proteins contained in salmons were carried out as follows. The proteins were extracted by adding a solubilizer (Mammalian Lysis Buffer (MCLI), Sigma-Aldrich Co. LLC) to salmon meat. Then, a urea buffer was added thereto to obtain a liquid protein extract. The constituents of the urea buffer are as mentioned below.
30 mM Tris
2 M thiourea
7 M urea
4% (w/v) CHAPS:
3-[(3-cholamidopropyl)dimethylammonio]propanesulfonate

### Moderate Amount of Dilute Hydrochloric Acid

The total amount was adjusted to 100 mL by the addition of distilled water. The pH was 8.5.

Then, 25 μg (by weight) each of the proteins was mixed to obtain a liquid extract.

Thereafter, the precipitation procedure was repeated twice using a 2D-CleanUP Kit (produced by GE). In the first round of precipitation, the collected liquid protein extract was precipitated by adding TCA (trichloroacetic acid) thereto and the precipitated product produced by this procedure (TCA-precipitated product) was collected. In the second round of precipitation, the TCA-precipitated product collected above was further precipitated by adding acetone thereto and the precipitated product (sample) produced by this procedure was collected.

### Preparation of a Sample Solution

Part of the collected sample (40 μg on a protein weight basis) was dissolved in 150 μL of a DeStreak Rehydration Solution (produced by GE), which is a swelling buffer for first-dimensional isoelectric focusing gels, thereby obtaining a sample solution for first-dimensional isoelectric focusing (sample solution for swelling). The constituents of the DeStreak Rehydration Solution are as mentioned below.
7M thiourea
2M urea
4% (w/v) of CHAPS
0.5% (v/v) IPG buffer; produced by GE
Moderate amount of BPB (bromophenol blue)
Penetration of the sample into first-dimensional isoelectric focusing gels First-dimensional isoelectric focusing gel strips (Immobiline Drystrip IPG gels (pH3-10NL); produced by GE) were immersed in 140 μL of the foregoing sample solution for first-dimensional isoelectric focusing (sample solution for swelling) and impregnated with the solution at room temperature overnight.

In this example, an IPGphor electrophoresis system produced by GE was used.

An electrophoresis tray was filled with silicone oil. Filter paper moisten with water was positioned at both ends of the gel strips impregnated with the sample, and the gel strips were set in the electrophoresis tray such that the gel strips were covered with silicone oil. Electrodes were placed on the gel strips with the filter paper intervening therebetween.

The maximum current of the isoelectric focusing system was set to 75 μA per gel strip, and the first-dimensional isoelectric focusing was carried out according to the following voltage program: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

### SDS Equilibration of Isoelectric Focusing Gels

After the aforementioned first-dimensional isoelectric focusing was done, the gel strips were taken out of the isoelectric focusing system, immersed in an equilibration buffer containing a reducing agent, and shaken at room temperature for 15 minutes. The constituents of the equilibration buffer containing the reducing agent are as mentioned below.
100 mM Tris-HCl (pH 8.0)
6M urea
30% (v/v) glycerol
2% (w/v) SDS
1% (w/v) DTT

Next, the equilibration buffer containing the reducing agent was removed, and then the gel strips were immersed in an equilibration buffer containing an alkylating agent and shaken at room temperature for 15 minutes to obtain SDS-equilibrated gels. The constituents of the equilibration buffer containing the alkylating agent are as mentioned below.
100 mM Tris-HCl (pH 8.0)
6M urea
30% (v/v) glycerol
2% (w/v) SDS
2.5% (w/v) iodoacetamide

### Second-Dimensional SDS-PAGE

In this example, the XCell SureLock Mini-Cell electrophoresis system produced by Life Technologies was used. The second-dimensional electrophoresis gels used were NuPAGE 4-12% Bis-Tris Gels produced by Life Technologies. Also, an electrophoresis buffer composed of the following constituents was prepared and used.
50 mM MOPS
50 mM Tris base
0.10% (w/v) SDS
1 mM EDTA

Further, an agarose solution for gel adhesion was used in this example, which was prepared by dissolving 0.5% (w/v) Agarose S (produced by Nippon Gene Co., Ltd.) and a moderate amount of BPB (bromophenol blue) in the electrophoresis buffer.

SDS-PAGE wells were washed well with the electrophoresis buffer, and then the buffer used for the washing was removed. Next, the washed wells were charged with the fully dissolved agarose solution for gel adhesion. Next, the SDS-equilibrated gel strips were immersed in agarose and closely adhered to second-dimensional electrophoresis gels using tweezers. After it was confirmed that agarose was fully fixed with the gels being closely adhered to each other, electrophoresis was performed at a constant voltage of 200 V for about 45 minutes.

### Fluorescent Staining of Gels

The gels were fluorescently stained with SYPRO Ruby (produced by Life Technologies).

First, an airtight container to be used was washed well in advance with 98% (v/v) ethanol. The electrophoresed second-dimensional electrophoresis gel strips were taken out of the SDS-PAGE system, placed onto the washed airtight container, and treated twice by immersion in 50% (v/v) methanol and 7% (v/v) aqueous solution containing acetic acid for 30 minutes. Then, a further immersion treatment was done for 10 minutes, with the solution being replaced by water. Next, the second-dimensional electrophoresis gel strips were immersed in 40 mL of SYPRO Ruby and shaken at room temperature overnight. Thereafter, the SYPRO Ruby was removed, and then the second-dimensional electrophoresis gel strips were washed with water and shaken in 10% (v/v) methanol and 7% (v/v) aqueous solution containing acetic acid for 30 minutes. Further shaking was done for at least 30 minutes, with the solution being replaced by water.

### Analysis

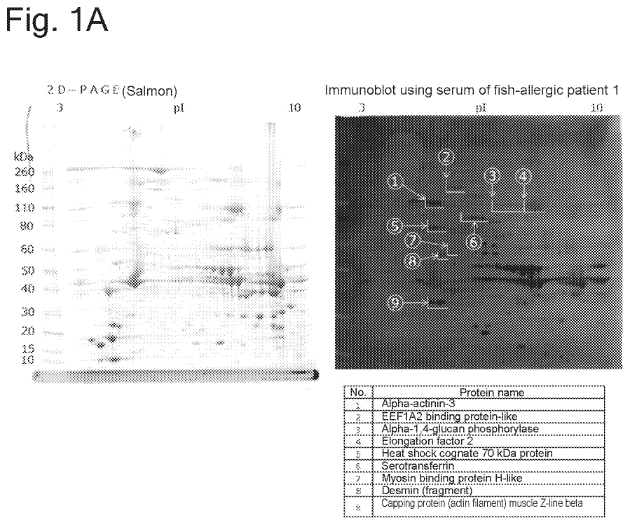

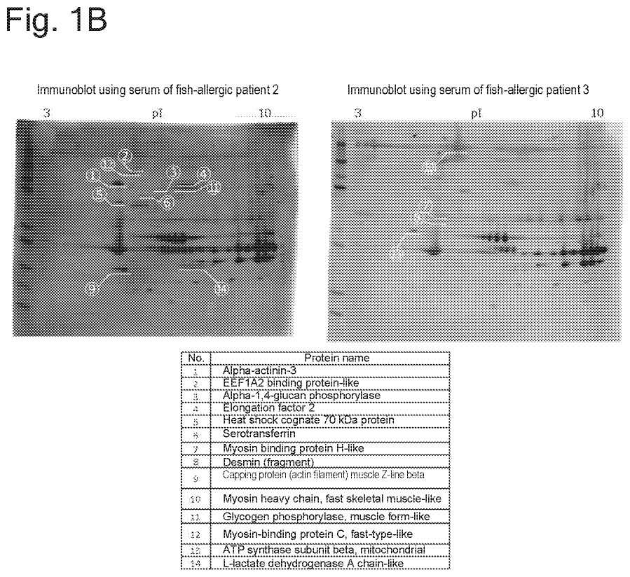

The second-dimensional electrophoresis gels obtained through the foregoing series of treatments were subjected to fluorescent image scanning on Typhoon9500 (produced by GE). The results of the two-dimensional electrophoresis as to the proteins contained in the salmon meat are shown in the left diagram FIG. 1A. Molecular weight marker bands are found at the left of the photograph of the gel. The positions of the bands denote particular molecular weights (in KDa).

### Example 2: Identification of Antigens by Immunoblotting (1)

Identification of antigens by immunoblotting was carried out by taking all the steps up to the step of “Second-dimensional SDS-PAGE” as described above in Example 1, followed by the steps of “Transfer to membrane”, “Immunoblotting” and “Analysis” as described below.

### Transfer to Membrane

Transfer to membrane was done using the following transfer system and transfer buffer.
Transfer system: XCell SureLock Mini-Cell and XCell II Blot Module (produced by Life Technologies)
Transfer buffer: NuPAGE Transfer Buffer (X20) (produced by Life Technologies), used in a form diluted 20-fold with milliQ water.

To be specific, proteins in the second-dimensional electrophoresis gels were transferred to a membrane (PVDF membrane) according to the following procedure.
(1) The PVDF membrane was immersed in 100% methanol followed by milliQ water, and then moved into the transfer buffer to hydrophilize the PVDF membrane.
(2) After sponge, filter paper, the second-dimensional electrophoresis gels treated by second-dimensional SDS-PAGE, the hydrophilized PVDF membrane, filter paper, and sponge were put in place in this order, the transfer system was energized at a constant voltage of 30 V for one hour.

### Immunoblotting

Immunoblotting of the membrane was carried out using, as a primary antibody, a serum sample from patient 1 with a fish allergy or a serum sample from a non-fish-allergic subject. This fish-allergic patient 1 was a patient diagnosed with immediate-type allergy to fish. This patient developed allergy symptoms to salmon, horse mackerel, conger, blackhead seabream, mackerel, sea bream, cod, and amberjack and exhibited positivity in the prick test.

Immunoblotting of the membrane was carried out according to the following procedure.
(1) The transferred membrane was shaken in a 5% skim milk/PBST solution (a PBS buffer containing 0.1% Tween 20 nonionic surfactant) at room temperature for one hour.
(2) The membrane was left to stand in a solution of 5% primary antibody serum in 5% skim milk/PBST at room temperature for one hour.
(3) The membrane was washed with a PBST solution (5 min.×3 times).
(4) The membrane was left to stand in a 1:5000 dilution of the secondary antibody, anti-human IgE-HRP (horseradish peroxidase), with a 5% skim milk/PBST solution at room temperature for one hour.
(5) The membrane was washed with a PBST solution (5 min.×3 times).
(6) The membrane was left to stand in Pierce Western Blotting Substrate Plus (produced by Thermo Fisher Scientific) for 5 minutes.

### Analysis

The membrane obtained through the foregoing series of treatments was subjected to fluorescent image scanning on Typhoon9500 (produced by GE).

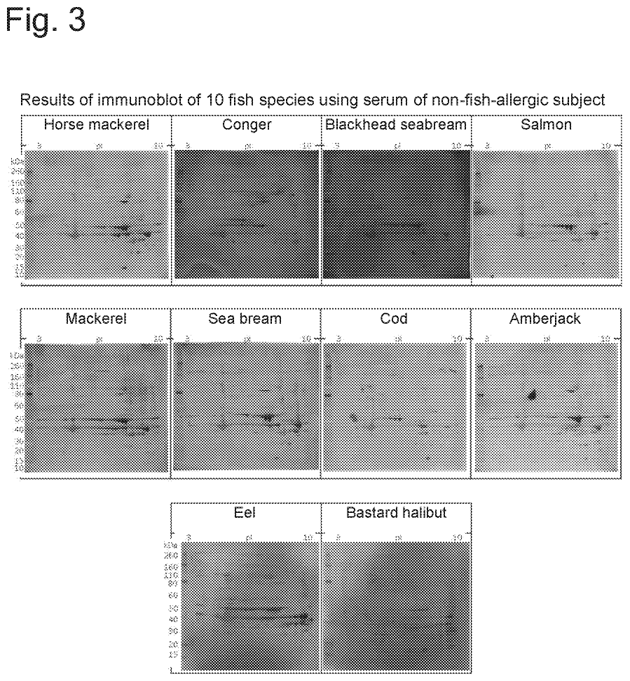

The immunoblots obtained with the serum from the fish-allergic patient 1 were compared with those obtained with the control serum from the non-fish-allergic subject. By immunoblotting of the proteins contained in the salmon meat using the serum from the fish-allergic patient 1 (the right diagram of FIG. 1A), spots were detected, which are different from the spots detected when the serum of the non-fish-allergic subject was used (FIG. 3) and different from those of the known salmon allergen proteins. The detected spots are shown in the immunoblot (the right diagram of FIG. 1A).

The molecular weights and isoelectric points of the 9 spots are as follows.
Spot 1: Molecular weight 90 to 120 kDa, pI 3.0 to 6.0
Spot 2: Molecular weight 120 to 160 kDa, pI 4.0 to 7.0
Spot 3: Molecular weight 90 to 120 kDa, pI 5.0 to 8.0
Spot 4: Molecular weight 90 to 120 kDa, pI 6.0 to 8.0
Spot 5: Molecular weight 60 to 90 kDa, pI 3.0 to 6.0
Spot 6: Molecular weight 70 to 100 kDa, pI 5.0 to 7.0
Spot 7: Molecular weight 40 to 70 kDa, pI 4.0 to 7.0
Spot 8: Molecular weight 40 to 70 kDa, pI 4.0 to 7.0
Spot 9: Molecular weight 20 to 50 kDa, pI 4.0 to 7.0

### Example 3: Mass Spectrometry and Identification of Antigens (1)

The amino acid sequences of the antigens that form the 9 protein spots were identified by mass spectroscopy.

To be specific, protein extraction and mass spectroscopy were done by the following procedure.
(1) Salmon meat was subjected to protein extraction, two-dimensional electrophoresis and transfer to membrane by following the procedures described in Example 1 and 2, and the resulting membrane was stained by shaking in a solution of 0.008% Direct blue in 40% ethanol and 10% acetic acid.
(2) Then, the membrane was decolorized by repeating a 5-minute treatment with 40% ethanol and 10% acetic acid three times, washed with water for 5 minutes, and then dried by air.
(3) A protein spot of interest was cut out with a clean cutter blade and put into a centrifugal tube. The cut membrane was subjected to hydrophilization with 50 μL of methanol, followed by washing with 100 μL of water twice and then centrifugal cleaning. Thereafter, 20 μL of 20 mM NH₄HCO₃ and 50% acetonitrile were added.
(4) 1 μL of 1 pmol/μL lysyl endopeptidase (produced by WAKO) was added, and the solution was left to stand at 37° C. for 60 minutes and then collected in a new centrifugal tube. After 20 μL of 20 mM NH₄HCO₃ and 70% acetonitrile were added to the membrane, the membrane was immersed therein at room temperature for 10 minutes, and the resulting solution was further collected. The solution was dissolved with 0.1% formic acid and 10 μL of 4% acetonitrile and transferred to a tube.
(5) The collected solution was dried under reduced pressure, dissolved with 15 μL of solution A (a 0.1% formic acid/4% acetonitrile solution), and analyzed by mass spectroscopy (ESI-TOF6600, produced by AB Sciex).
(6) Identification of proteins based on the MS data obtained with the mass spectrometer was done by searching the NCBI or UniProt database.

### Results

The mass spectrometry of the spots resulted in the detection of the following amino acid sequences.
Spot 1: Amino acid sequence of SEQ ID NO: 3
Spot 2: Amino acid sequences of SEQ ID NOs: 6-8
Spot 3: Amino acid sequences of SEQ ID NOs: 11-17
Spot 4: Amino acid sequences of SEQ ID NOs: 20-24
Spot 5: Amino acid sequences of SEQ ID NOs: 27-31
Spot 6: Amino acid sequences of SEQ ID NOs: 34-41
Spot 7: Amino acid sequences of SEQ ID NOs: 44-54
Spot 8: Amino acid sequences of SEQ ID NOs: 57-59
Spot 9: Amino acid sequences of SEQ ID NOs: 62-68

Furthermore, the spots were identified as the following proteins by the analysis of the mass data obtained from the mass spectrometer for the spots at NCBI for spot 1 and at UniProt for spots 2 to 9.
Spot 1: Alpha-actinin-3 (NCBI protein accession number: XP_014051545.1, DNA accession number: XM_014196070.1) (amino acid sequence: SEQ ID NO: 2, encoding nucleotide sequence: SEQ ID NO: 1)
Spot 2: EEF1A2 binding protein-like (UniProt protein accession number: B5RI29, DNA accession number: ACH85270.1) (amino acid sequence: SEQ ID NO: 5, encoding nucleotide sequence: SEQ ID NO: 4)
Spot 3: Alpha-1,4-glucan phosphorylase (UniProt protein accession number: B5DG55, DNA accession number: ACH70729.1) (amino acid sequence: SEQ ID NO: 10, encoding nucleotide sequence: SEQ ID NO: 9)
Spot 4: Elongation factor 2 (UniProt protein accession number: C0H9N2, DNA accession number: ACN10751.1) (amino acid sequence: SEQ ID NO: 19, encoding nucleotide sequence: SEQ ID NO: 18)
Spot 5: Heat shock cognate 70 kDa protein (UniProt protein accession number: B5X3U6, DNA accession number: ACI33977.1) (amino acid sequence: SEQ ID NO: 26, encoding nucleotide sequence: SEQ ID NO: 25)
Spot 6: Serotransferrin (UniProt protein accession number: P79815, DNA accession number: BAA13759.1) (amino acid sequence: SEQ ID NO: 33, encoding nucleotide sequence: SEQ ID NO: 32)
Spot 7: Myosin binding protein H-like (UniProt protein accession number: B5DG45, DNA accession number: ACH70719.1) (amino acid sequence: SEQ ID NO: 43, encoding nucleotide sequence: SEQ ID NO: 42)
Spot 8: Desmin (fragment) (UniProt protein accession number: Q8UWF1, DNA accession number: CAC83053.1) (amino acid sequence: SEQ ID NO: 56, encoding nucleotide sequence: SEQ ID NO: 55)
Spot 9: Capping protein (actin filament) muscle Z-line beta (UniProt protein accession number: B5DFX6, DNA accession number: ACH70650.1) (amino acid sequence: SEQ ID NO: 61, encoding nucleotide sequence: SEQ ID NO: 60)

### Example 4: Identification of Antigens in Other Fish Species (1)

Identification of antigens were carried out by the same procedure as described in Example 1 and Example 2 using meat of 9 fish species other than salmon, i.e., horse mackerel, conger, blackhead seabream, mackerel, sea bream, cod, amberjack, eel, and bastard halibut.

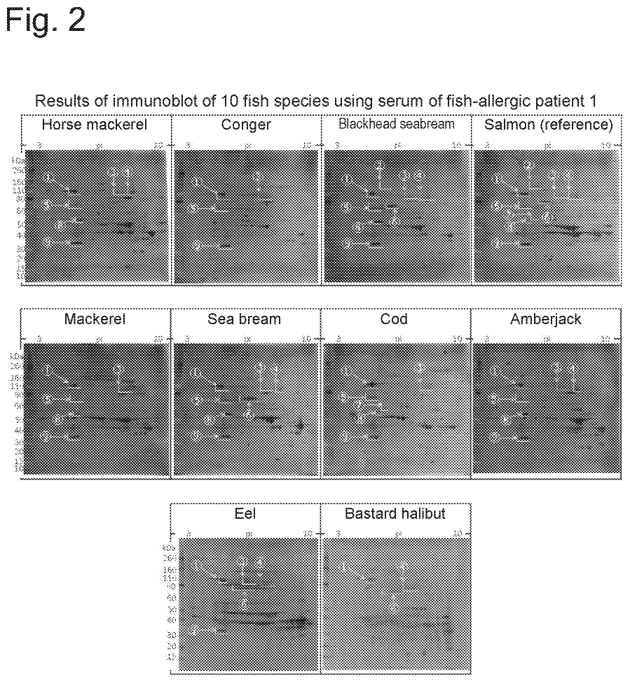

The immunoblots obtained with the serum from the fish-allergic patient 1 were compared with those obtained with the control serum from the non-fish-allergic subject. By immunoblotting of the proteins contained in the meat of each of the 9 fish species using the serum from the fish-allergic patient 1 (FIG. 2), spots were detected, which are different from the spots detected when the serum of the non-fish-allergic subject was used (FIG. 3) and corresponded to some of the 9 spots obtained in salmon.

The following spots were detected in each of the fish species.
Horse mackerel: Spots 1, 3 to 5, 8, and 9
Conger: Spots 1, 3, 5, and 9
Blackhead seabream: Spots 1 to 6, 8, and 9
Mackerel: Spots 1, 3, 5, 8, and 9
Sea bream: Spots 1, 3 to 6, 8, and 9
Cod: Spots 1, 3, 5, and 7 to 9
Amberjack: Spots 1, 3 to 5, 8, and 9
Eel: Spots 1, 3, 4, 6, and 9
Bastard halibut: Spots 1, 4, and 6

### Example 5: Identification of Antigens by Immunoblotting (2)

The immunoblots obtained with the serum from a fish-allergic patient were compared with those obtained with the control serum from the non-fish-allergic subject in the same manner as in Example 2. By immunoblotting of the proteins contained in the salmon meat using the serum from the fish-allergic patient 2 (the left diagram of FIG. 1), 3 spots (spots 11, 12 and 14) were newly detected, which are different from the spots detected when the serum of the non-fish-allergic subject was used (FIG. 3) and different from those of the known salmon allergen proteins, in addition to 7 (spots 1 to 6 and 9) out of the 9 spots detected in Example 2. A total of 10 spots are shown in the immunoblot (the left diagram of FIG. 1).

The molecular weights and isoelectric points of the newly detected 3 spots are as follows.
Spot 11: Molecular weight 90 to 110, pI 6.5 to 7.0
Spot 12: Molecular weight 120 to 140, pI 5.0 to 6.0
Spot 14: Molecular weight 30 to 40, pI 6.5 to 7.5

### Example 6: Mass Spectrometry and Identification of Antigens (2)

The amino acid sequences of the antigens that form the newly detected 3 protein spots were identified by mass spectroscopy in the same manner as in Example 3.

The mass spectrometry of the spots resulted in the detection of the following amino acid sequences.
Spot 11: Amino acid sequences of SEQ ID NOs: 111-119
Spot 12: Amino acid sequences of SEQ ID NOs: 122-136
Spot 14: Amino acid sequences of SEQ ID NOs: 145-149

Furthermore, the spots were identified as the following proteins by the analysis of the mass data obtained from the mass spectrometer for the spots at NCBI.
Spot 11: Glycogen phosphorylase, muscle form-like (NCBI protein accession number: XP_013984904.1, DNA accession number: XM_014129429.1) (amino acid sequence: SEQ ID NO: 110, encoding nucleotide sequence: SEQ ID NO: 109)
Spot 12: Myosin-binding protein C, fast-type-like (NCBI protein accession number: XP_014014310.1, DNA accession number: XM_014158835.1) (amino acid sequence: SEQ ID NO: 121, encoding nucleotide sequence: SEQ ID NO: 120)
Spot 14: L-lactate dehydrogenase A chain-like (NCBI protein accession number: XP_014003141.1, DNA accession number: XM_014147666.1) (amino acid sequence: SEQ ID NO: 144, encoding nucleotide sequence: SEQ ID NO: 143)

### Example 7: Identification of Antigens by Immunoblotting (3)

The immunoblots obtained with the serum from a fish-allergic patient were compared with those obtained with the control serum from the non-fish-allergic subject in the same manner as in Example 2. By immunoblotting of the proteins contained in the salmon meat using the serum from the fish-allergic patient 3 (the right diagram of FIG. 1), 2 spots (spots 10 and 13) were newly detected, which are different from the spots detected when the serum of the non-fish-allergic subject was used (FIG. 3) and different from those of the known salmon allergen proteins, in addition to 2 (spots 7 and 8) out of the 9 spots detected in Example 2. A total of 4 spots are shown in the immunoblot (the right diagram of FIG. 1).

The molecular weights and isoelectric points of the newly detected 2 spots are as follows.
Spot 10: Molecular weight 200 to 230, pI 4.5 to 5.5
Spot 13: Molecular weight 45 to 55, pI 4.0 to 5.5

### Example 8: Mass Spectrometry and Identification of Antigens (3)

The amino acid sequences of the antigens that form the newly detected 2 protein spots were identified by mass spectroscopy in the same manner as in Example 3.

The mass spectrometry of the spots resulted in the detection of the following amino acid sequences.
Spot 10: Amino acid sequences of SEQ ID NOs: 71-108
Spot 13: Amino acid sequences of SEQ ID NOs: 139-142

Furthermore, the spots were identified as the following proteins by the analysis of the mass data obtained from the mass spectrometer for the spots at NCBI.
Spot 10: Myosin heavy chain, fast skeletal muscle-like (NCBI protein accession number: XP_014039990.1, DNA accession number: XM_014184515.1) (amino acid sequence: SEQ ID NO: 70, encoding nucleotide sequence: SEQ ID NO: 69)
Spot 13: ATP synthase subunit beta, mitochondrial (NCBI protein accession number: XP_014007238.1, DNA accession number: XM_014151763.1) (amino acid sequence: SEQ ID NO: 138, encoding nucleotide sequence: SEQ ID NO: 137)

### Example 9: Identification of Antigens in Other Fish Species (2)

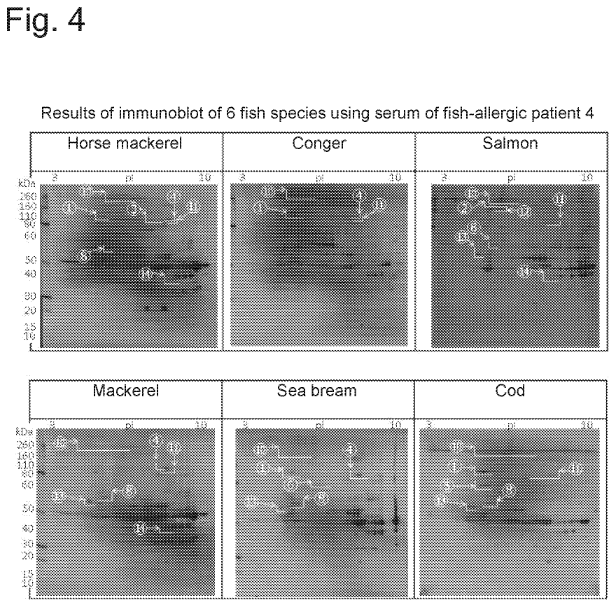

Identification of antigens were carried out in the same manner as in Example 4 using meat of 6 fish species, i.e., horse mackerel, conger, salmon, mackerel, sea bream, and cod. The immunoblots obtained with the serum from the fish-allergic patient 4 were compared with those obtained with the control serum from the non-fish-allergic subject. By immunoblotting of the proteins contained in the meat of each of the 6 fish species using the serum from the fish-allergic patient 4 (FIG. 4), spots were detected, which are different from the spots detected when the serum of the non-fish-allergic subject was used (FIG. 3) and corresponded to some of a total of 14 spots obtained with the serum from the fish-allergic patients 1 to 3.

The following spots were detected in each of the fish species.
Horse mackerel: Spots 1, 3, 4, 8, 10, 11, and 14
Conger: Spots 1, 4, 10, and 11
Salmon: Spots 2, 8, and 10 to 14
Mackerel: Spots 4, 8, 10, 11, 13, and 14
Sea bream: Spots 1, 4, 6, 8, 10, and 13
Cod: Spots 1, 5, 8, 10, 11, and 13

### Example 10: Identification of Epitopes

### Epitopes of Fish Allergen Components

Identification of epitopes was carried out by the following procedure as to the fish allergen components.

### (A) Epitope Mapping (1)

Epitope mapping was carried out using a library of overlapping peptides (length: 15 amino acids) corresponding to the amino acid sequences identified as allergy components of fish and the amino acid sequences of known fish allergy antigens aldolase, β-enolase, and glyceraldehyde-3-phosphate dehydrogenase (Non Patent Literature 2-4). Specifically, the library of overlapping peptides was prepared on the basis of the amino acid sequences of SEQ ID NO: 2 for alpha-actinin-3, SEQ ID NO: 5 for EEF1A2 binding protein-like, SEQ ID NO: 10 for alpha-1,4-glucan phosphorylase, SEQ ID NO: 19 for elongation factor 2, SEQ ID NO: 26 for heat shock cognate 70 kDa protein, SEQ ID NO: 33 for serotransferrin, SEQ ID NO: 43 for myosin binding protein H-like, SEQ ID NO: 56 for desmin (fragment), SEQ ID NO: 61 for capping protein (actin filament) muscle Z-line beta, SEQ ID NO: 70 for myosin heavy chain, fast skeletal muscle-like, SEQ ID NO: 110 for glycogen phosphorylase, muscle form-like, SEQ ID NO: 121 for myosin-binding protein C, fast-type-like, SEQ ID NO: 138 for ATP synthase subunit beta, mitochondrial, SEQ ID NO: 144 for L-lactate dehydrogenase A chain-like, SEQ ID NO: 445 for aldolase, SEQ ID NO: 446 for β-enolase, and SEQ ID NO: 447 for glyceraldehyde-3-phosphate dehydrogenase.

The peptides to be synthesized were shifted by 10 amino acids. Thus, each peptide had an overlap of 5 amino acids with each of the peptides previous and subsequent thereto.

For preparation of peptide arrays, the Intavis CelluSpots™ technique was used. Specifically, the peptide arrays were prepared by the following procedure: (1) synthesizing peptides of interest on amino-modified cellulose disks using an automated chemical synthesis apparatus (Intavis MultiPep RS), (2) dissolving the amino-modified cellulose disks to obtain a cellulose-bound peptide solution, and (3) spotting the cellulose-bound peptides onto coated glass slides. The details of each procedure are as described below.

### (1) Synthesis of Peptide

Peptide synthesis was carried out in incremental steps through 9-fluorenylmethoxycarbonyl (Fmoc) chemical reaction on amino-modified cellulose disks in 384-well synthesis plates. Specifically, amino acids in which a Fmoc group is bonded to an amino group were activated in a solution of N,N′-diisopropylcarbodiimide (DIC) and 1-hydroxybenzotriazole (HOBt) in dimethylformamide (DMF) and added dropwise to the cellulose disks so that the Fmoc group-bound amino acids were bound to the amino groups on the cellulose disks (coupling). Unreacted amino groups were capped with acetic anhydride and washed with DMF. Furthermore, the Fmoc groups were eliminated from the amino groups of the amino acids bound to the amino groups on the cellulose disks by treatment with piperidine and washing with DMF. The amino acids bound to the amino groups on the cellulose disks were repetitively subjected to the coupling, the capping, and the Fmoc group elimination described above to elongate the amino terminus for peptide synthesis.

### (2) Dissolution of Amino-Modified Cellulose Disk

The peptides-bound cellulose disks of interest obtained above in the subsection titled “(1) Synthesis of peptide” were transferred to 96-well plates and treated with a side chain deprotection mixed solution of trifluoroacetic acid (TFA), dichloromethane, triisopropylsilane (TIPS), and water for deprotection of amino acid side chains. Then, the deprotected cellulose-bound peptides were dissolved in a mixed solution of TFA, perfluoromethanesulfonic acid (TFMSA), TIPS, and water and precipitated in tetrabutyl methyl ether (TBME), and the precipitate was resuspended in dimethyl sulfoxide (DMSO) and mixed with a mixed solution of NaCl, sodium citrate, and water to obtain a peptide solution for slide spotting.

### (3) Spotting of Cellulose-Bound Peptide Solution

The peptide solution for slide spotting obtained above in the subsection titled “(2) Dissolution of amino-modified cellulose disk” was spotted onto Intavis CelluSpots™ slides using Intavis Slide Spotting Robot, and the slides were dried to prepare peptide arrays.

The presence or absence of binding, to each peptide fragment, of an IgE antibody present in the serum of a fish-allergic patient was measured through antigen-antibody reaction using the peptide arrays. The measurement was carried out according to the following procedure.
(1) The peptides were shaken at room temperature for 1 hour in PBST.
(2) The peptide arrays were shaken at overnight at room temperature in 5% serum/PBST.
(3) The peptide arrays were washed with a PBST solution (a PBS buffer containing 0.1% Tween 20 nonionic surfactant) for 5 minutes (×3).
(4) Anti-human IgE antibody-HRP (1:5,000, PBST) was added thereto, and the peptide arrays were shaken at room temperature for 1 hour.
(5) The peptide arrays were washed with a PBST solution for 5 minutes (×3).
(6) Pierce ECL Plus Western Blotting Substrate (produced by Thermo Fisher Scientific) was added thereto, and the peptide arrays were shaken at room temperature for 5 minutes.
(7) The chemiluminescence of the peptides treated as described above in (1) to (6) was measured using Amersham Imager 600.

Chemiluminescence intensity in images obtained by the measurement described above in (7) was converted into a numeric value using ImageQuant TL (GE Healthcare). The average value of numeric values determined from images obtained from results about the serum of 5 patients was divided by the average value of numeric values determined from images obtained from results about the serum of 4 non-fish-allergic subjects. It was determined that a peptide having a calculated value (average value of the patient group/average value of the non-fish-allergic subject group) of at least 1.5 and a significant difference of at least 0.05 in the Mann-Whitney U test was a peptide bound to an IgE antibody in a patient-specific manner.

### (B) Epitope Mapping (2): Overlapping

On the basis of the sequences (SEQ ID NOs: 150-154 for alpha-actinin-3, SEQ ID NO: 155 for EEF1A2 binding protein-like, SEQ ID NOs: 156 and 157 for alpha-1,4-glucan phosphorylase, SEQ ID NOs: 158 and 159 for elongation factor 2, SEQ ID NOs: 160-162 for heat shock cognate 70 kDa protein, SEQ ID NOs: 163-167 for serotransferrin, SEQ ID NOs: 168-171 for myosin binding protein H-like, SEQ ID NOs: 172-174 for desmin (fragment), SEQ ID NOs: 175-178 for capping protein (actin filament) muscle Z-line beta, SEQ ID NOs: 179-185 for myosin heavy chain, fast skeletal muscle-like, SEQ ID NO: 186 for glycogen phosphorylase, muscle form-like, SEQ ID NOs: 187-196 for myosin-binding protein C, fast-type-like, SEQ ID NO: 197 for ATP synthase subunit beta, mitochondrial, SEQ ID NO: 198 for L-lactate dehydrogenase A chain-like, SEQ ID NO: 199 for aldolase, SEQ ID NOs: 200-202 for β-enolase, and SEQ ID NOs: 203 and 204 for glyceraldehyde-3-phosphate dehydrogenase) of the peptides bound to an IgE antibody in serum in a patient-specific manner as described above in (A), a library of overlapping peptide fragments (length: 10 amino acids) was prepared using the sequences of the peptides and sequences in which sequences were added so as to flank each peptide in the amino acid sequences of allergen components (SEQ ID NO: 2 for alpha-actinin-3, SEQ ID NO: 5 for EEF1A2 binding protein-like, SEQ ID NO: 10 for alpha-1,4-glucan phosphorylase, SEQ ID NO: 19 for elongation factor 2, SEQ ID NO: 26 for heat shock cognate 70 kDa protein, SEQ ID NO: 33 for serotransferrin, SEQ ID NO: 43 for myosin binding protein H-like, SEQ ID NO: 56 for desmin (fragment), SEQ ID NO: 61 for capping protein (actin filament) muscle Z-line beta, SEQ ID NO: 70 for myosin heavy chain, fast skeletal muscle-like, SEQ ID NO: 110 for glycogen phosphorylase, muscle form-like, SEQ ID NO: 121 for myosin-binding protein C, fast-type-like, SEQ ID NO: 138 for ATP synthase subunit beta, mitochondrial, SEQ ID NO: 144 for L-lactate dehydrogenase A chain-like, SEQ ID NO: 445 for aldolase, SEQ ID NO: 446 for β-enolase, and SEQ ID NO: 447 for glyceraldehyde-3-phosphate dehydrogenase) comprising the sequences of the peptide. Epitope mapping was carried out using the library.

The peptides to be synthesized were shifted by one amino acid. Thus, each peptide had an overlap of 9 amino acids with each of the peptides previous and subsequent thereto.

The library was prepared by the same procedure as described above in (A), and the presence or absence of binding of an IgE antibody present in the serum of a patient and a non-fish-allergic subject to each peptide fragment was measured by the same technique as described above. Amino acids at positions where the binding activity against IgE antibodies from patients were lost or decreased in the peptides shifted by one amino acid while binding activity against IgE antibodies from non-fish-allergic subjects arose were confirmed as amino acids adversely affecting the specificity of binding to IgE antibodies.

Chemiluminescence intensity in images obtained by measurement was converted into a numeric value in the same manner as in (A) using ImageQuant TL (GE Healthcare). When values obtained using the sequences (SEQ ID NOs: 150-204) that were of the peptides confirmed to bind to an IgE antibody in a patient specific manner from the images obtained from results about the serum of 5 patients and served as the basis of overlapping were defined as 100%, it was determined that: a peptide having a value of less than 20% had no binding activity against IgE antibodies; a peptide having a value of 20% or more and less than 50% had binding activity, albeit poor, against IgE antibodies; a peptide having a value of 50% or more and less than 70% had binding activity, albeit somewhat poor, against IgE antibodies; a peptide having a value of 70% or more and less than 90% had equivalent binding activity against IgE antibodies; and a peptide having a value of 90% or more had almost equivalent or good binding activity against IgE antibodies and was thus a peptide had remaining binding activity against IgE antibodies.

This analysis found regions important for binding to IgE antibodies from patients, in the sequences serving as the basis of overlapping.

### (C) Epitope Mapping (3): Alanine Scanning

From the amino acid sequences identified above in (A), a library of peptide fragments in which N-terminal amino acids were substituted one by one by alanine according to a technique called alanine scanning (Non Patent Literature 5) was prepared by the same technique as described above. The presence or absence of binding of an IgE antibody present in the serum of a patient and a non-fish-allergic subject to each peptide fragment was measured by the same technique as described above. Amino acids at positions where the binding activity against IgE antibodies from patients were lost or decreased by the substitution by alanine while binding activity against IgE antibodies from non-fish-allergic subjects arose were confirmed as amino acids important for exertion of original antigenicity, or amino acids influencing exertion of original antigenicity. Also, amino acids at positions where the binding activity against IgE antibodies from patients was maintained even after the substitution by alanine while binding activity against IgE antibodies from non-fish-allergic subjects did not arise were confirmed as substitutable positions that were not important for exertion of original antigenicity,

Chemiluminescence intensity in images obtained by measurement was converted into a numeric value in the same manner as in (A) using ImageQuant TL (GE Healthcare). When values obtained using the sequences (SEQ ID NOs: 150-204) that were of the peptides confirmed to bind to an IgE antibody in a patient specific manner from the images obtained from results about the serum of 5 patients and served as the basis of overlapping were defined as 100%, it was determined that: a peptide having a value of less than 20% had no binding activity against IgE antibodies; a peptide having a value of 20% or more and less than 50% had binding activity, albeit poor, against IgE antibodies; a peptide having a value of 50% or more and less than 70% had binding activity, albeit somewhat poor, against IgE antibodies; a peptide having a value of 70% or more and less than 90% had equivalent binding activity against IgE antibodies; and a peptide having a value of 90% or more had almost equivalent or good binding activity against IgE antibodies and was thus a peptide had remaining binding activity against IgE antibodies.

By this analysis, common sequences important for exertion of original antigenicity were found in regions important for binding to IgE antibodies from patients, in the sequences serving as the basis of overlapping.

### (D) Results

As a result of the epitope mapping described above in (A), an IgE antibody was confirmed to bind in a patient-specific manner to each of epitope Nos. 1-5 (peptides having the amino acid sequences of SEQ ID NOs: 150-154, respectively) for alpha-actinin-3, epitope No. 6 (a peptide having the amino acid sequence of SEQ ID NO: 155) for EEF1A2 binding protein-like, epitope Nos. 7 and 8 (peptides having the amino acid sequences of SEQ ID NOs: 156 and 157, respectively) for alpha-1,4-glucan phosphorylase, epitope Nos. 9 and 10 (peptides having the amino acid sequences of SEQ ID NOs: 158 and 159, respectively) for elongation factor 2, epitope Nos. 11-13 (peptides having the amino acid sequences of SEQ ID NOs: 160-162, respectively) for heat shock cognate 70 kDa protein, epitope Nos. 14-18 (peptides having the amino acid sequences of SEQ ID NOs: 163-167, respectively) for serotransferrin, epitope Nos. 19-22 (peptides having the amino acid sequences of SEQ ID NOs: 168-171, respectively) for myosin binding protein H-like, epitope Nos. 23-25 (peptides having the amino acid sequences of SEQ ID NOs: 172-174, respectively) for desmin (fragment), epitope Nos. 26-29 (peptides having the amino acid sequences of SEQ ID NOs: 175-178, respectively) for capping protein (actin filament) muscle Z-line beta, epitope Nos. 30-36 (peptides having the amino acid sequences of SEQ ID NOs: 179-185, respectively) for myosin heavy chain, fast skeletal muscle-like, epitope No. 37 (a peptide having the amino acid sequence of SEQ ID NO: 186) for glycogen phosphorylase, muscle form-like, epitope Nos. 38-47 (peptides having the amino acid sequences of SEQ ID NOs: 187-196, respectively) for myosin-binding protein C, fast-type-like, epitope No. 48 (a peptide having the amino acid sequence of SEQ ID NO: 197) for ATP synthase subunit beta, mitochondrial, epitope No. 49 (a peptide having the amino acid sequence of SEQ ID NO: 198) for L-lactate dehydrogenase A chain-like, epitope No. 50 (a peptide having the amino acid sequence of SEQ ID NO: 199) for aldolase, epitope Nos. 51-53 (peptides having the amino acid sequences of SEQ ID NOs: 200-202, respectively) for β-enolase, and epitope Nos. 54 and 55 (peptides having the amino acid sequences of SEQ ID NOs: 203 and 204, respectively) for glyceraldehyde-3-phosphate dehydrogenase. Portions to which the peptide sequences of these epitopes correspond in the sequences of the allergen components identified in Examples 2, 3 and 5 to 8 and the known allergen components aldolase, β-enolase, and glyceraldehyde-3-phosphate dehydrogenase are shown in Table 2.

TABLE 2

| Epitope | | Sequence of | |
|---|---|---|---|
| No. | Protein name | epitope | Position in sequence of allergen component |
| 1 | Alpha-actinin-3 | SEQ ID NO: 150 | Amino acids 81-95 of SEQ ID NO: 2 |
| 2 | Alpha-actinin-3 | SEQ ID NO: 151 | Amino acids 201-215 of SEQ ID NO: 2 |
| 3 | Alpha-actinin-3 | SEQ ID NO: 152 | Amino acids 251-265 of SEQ ID NO: 2 |
| 4 | Alpha-actinin-3 | SEQ ID NO: 153 | Amino acids 351-365 of SEQ ID NO: 2 |
| 5 | Alpha-actinin-3 | SEQ ID NO: 154 | Amino acids 841-855 of SEQ ID NO: 2 |
| 6 | EEF1A2 binding | SEQ ID NO: 155 | Amino acids 1021-1035 of SEQ ID NO: 5 |
| | protein-like | | |
| 7 | Alpha-1,4-glucan | SEQ ID NO: 156 | Amino acids 261-275 of SEQ ID NO: 10 |
| | phosphorylase | | |
| 8 | Alpha-1,4-glucan | SEQ ID NO: 157 | Amino acids 771-785 of SEQ ID NO: 10 |
| | phosphorylase | | |
| 9 | Elongation factor 2 | SEQ ID NO: 158 | Amino acids 11-25 of SEQ ID NO: 19 |
| 10 | Elongation factor 2 | SEQ ID NO: 159 | Amino acids 31-45 of SEQ ID NO: 19 |
| 11 | Heat shock cognate 70 | SEQ ID NO: 160 | Amino acids 121-135 of SEQ ID NO: 26 |
| | kDa protein | | |
| 12 | Heat shock cognate 70 | SEQ ID NO: 161 | Amino acids 521-535 of SEQ ID NO: 26 |
| | kDa protein | | |
| 13 | Heat shock cognate 70 | SEQ ID NO: 162 | Amino acids 561-575 of SEQ ID NO: 26 |
| | kDa protein | | |
| 14 | Serotransferrin | SEQ ID NO: 163 | Amino acids 101-115 of SEQ ID NO: 33 |
| 15 | Serotransferrin | SEQ ID NO: 164 | Amino acids 201-215 of SEQ ID NO: 33 |
| 16 | Serotransferrin | SEQ ID NO: 165 | Amino acids 271-285 of SEQ ID NO: 33 |
| 17 | Serotransferrin | SEQ ID NO: 166 | Amino acids 321-335 of SEQ ID NO: 33 |
| 18 | Serotransferrin | SEQ ID NO: 167 | Amino acids 521-535 of SEQ ID NO: 33 |
| 19 | Myosin binding protein | SEQ ID NO: 168 | Amino acids 201-215 of SEQ ID NO: 43 |
| | H-like | | |
| 20 | Myosin binding protein | SEQ ID NO: 169 | Amino acids 261-275 of SEQ ID NO: 43 |
| | H-like | | |
| 21 | Myosin binding protein | SEQ ID NO: 170 | Amino acids 381-395 of SEQ ID NO: 43 |
| | H-like | | |
| 22 | Myosin binding protein | SEQ ID NO: 171 | Amino acids 441-455 of SEQ ID NO: 43 |
| | H-like | | |
| 23 | Desmin (fragment) | SEQ ID NO: 172 | Amino acids 101-115 of SEQ ID NO: 56 |
| 24 | Desmin (fragment) | SEQ ID NO: 173 | Amino acids 281-295 of SEQ ID NO: 56 |
| 25 | Desmin (fragment) | SEQ ID NO: 174 | Amino acids 431-445 of SEQ ID NO: 56 |
| 26 | Capping protein (actin | SEQ ID NO: 175 | Amino acids 131-145 of SEQ ID NO: 61 |
| | filament) muscle Z-line | | |
| | beta | | |
| 27 | Capping protein (actin | SEQ ID NO: 176 | Amino acids 151-165 of SEQ ID NO: 61 |
| | filament) muscle Z-line | | |
| | beta | | |
| 28 | Capping protein (actin | SEQ ID NO: 177 | Amino acids 231-245 of SEQ ID NO: 61 |
| | filament) muscle Z-line | | |
| | beta | | |
| 29 | Capping protein (actin | SEQ ID NO: 178 | Amino acids 251-265 of SEQ ID NO: 61 |
| | filament) muscle Z-line | | |
| | beta | | |
| 30 | Myosin heavy chain, fast | SEQ ID NO: 179 | Amino acids 61-75 of SEQ ID NO: 70 |
| | skeletal muscle-like | | |
| 31 | Myosin heavy chain, fast | SEQ ID NO: 180 | Amino acids 471-485 of SEQ ID NO: 70 |
| | skeletal muscle-like | | |
| 32 | Myosin heavy chain, fast | SEQ ID NO: 181 | Amino acids 621-635 of SEQ ID NO: 70 |
| | skeletal muscle-like | | |
| 33 | Myosin heavy chain, fast | SEQ ID NO: 182 | Amino acids 981-995 of SEQ ID NO: 70 |
| | skeletal muscle-like | | |
| 34 | Myosin heavy chain, fast | SEQ ID NO: 183 | Amino acids 1011-1025 of SEQ ID NO: 70 |
| | skeletal muscle-like | | |
| 35 | Myosin heavy chain, fast | SEQ ID NO: 184 | Amino acids 1041-1055 of SEQ ID NO: 70 |
| | skeletal muscle-like | | |
| 36 | Myosin heavy chain, fast | SEQ ID NO: 185 | Amino acids 1741-1755 of SEQ ID NO: 70 |
| | skeletal muscle-like | | |
| 37 | Glycogen phosphorylase, | SEQ ID NO: 186 | Amino acids 261-275 of SEQ ID NO: 110 |
| | muscle form-like | | |
| 38 | Myosin-binding protein | SEQ ID NO: 187 | Amino acids 181-195 of SEQ ID NO: 121 |
| | C, fast-type-like | | |
| 39 | Myosin-binding protein | SEQ ID NO: 188 | Amino acids 211-225 of SEQ ID NO: 121 |
| | C, fast-type-like | | |
| 40 | Myosin-binding protein | SEQ ID NO: 189 | Amino acids 221-235 of SEQ ID NO: 121 |
| | C, fast-type-like | | |
| 41 | Myosin-binding protein | SEQ ID NO: 190 | Amino acids 231-245 of SEQ ID NO: 121 |
| | C, fast-type-like | | |
| 42 | Myosin-binding protein | SEQ ID NO: 191 | Amino acids 251-265 of SEQ ID NO: 121 |
| | C, fast-type-like | | |
| 43 | Myosin-binding protein | SEQ ID NO: 192 | Amino acids 371-385 of SEQ ID NO: 121 |
| | C, fast-type-like | | |
| 44 | Myosin-binding protein | SEQ ID NO: 193 | Amino acids 491-505 of SEQ ID NO: 121 |
| | C, fast-type-like | | |
| 45 | Myosin-binding protein | SEQ ID NO: 194 | Amino acids 651-665 of SEQ ID NO: 121 |
| | C, fast-type-like | | |
| 46 | Myosin-binding protein | SEQ ID NO: 195 | Amino acids 831-845 of SEQ ID NO: 121 |
| | C, fast-type-like | | |
| 47 | Myosin-binding protein | SEQ ID NO: 196 | Amino acids 951-965 of SEQ ID NO: 121 |
| | C, fast-type-like | | |
| 48 | ATP synthase subunit | SEQ ID NO: 197 | Amino acids 211-225 of SEQ ID NO: 138 |
| | beta, mitochondrial | | |
| 49 | L-lactate dehydrogenase | SEQ ID NO: 198 | Amino acids 11-25 of SEQ ID NO: 144 |
| | A chain-like | | |
| 50 | Aldolase | SEQ ID NO: 199 | Amino acids 301-315 of SEQ ID NO: 445 |
| 51 | β-enolase | SEQ ID NO: 200 | Amino acids 51-65 of SEQ ID NO: 446 |
| 52 | β-enolase | SEQ ID NO: 201 | Amino acids 131-145 of SEQ ID NO: 446 |
| 53 | β-enolase | SEQ ID NO: 202 | Amino acids 191-205 of SEQ ID NO: 446 |
| 54 | Glyceraldehyde-3- | SEQ ID NO: 203 | Amino acids 41-55 of SEQ ID NO: 447 |
| | phosphate dehydrogenase | | |
| 55 | Glyceraldehyde-3- | SEQ ID NO: 204 | Amino acids 251-265 of SEQ ID NO: 447 |
| | phosphate dehydrogenase | | |

Amino acids important for binding of the epitope sequences to IgE antibodies from allergic patients were identified by the procedures of (B) and (C) mentioned above as to each of the epitopes.

### (1) Epitope 1

In epitope 1 (SEQ ID NO: 150), SEQ ID NO: 207 corresponding to the amino acids at positions 1-10 and SEQ ID NO: 210 corresponding to the amino acids at positions 9-14 were identified as regions important for binding to IgE antibodies from patients.

In the region of SEQ ID NO: 207 important for binding, the amino acids at positions 7-9 were identified as amino acids particularly important for binding to IgE antibodies from allergic patients, and the amino acids at positions 1 and 10 were identified as amino acids influencing binding to IgE antibodies from allergic patients. The amino acids at positions 2-6 of SEQ ID NO: 207 had no influence on the binding activity against IgE antibodies from allergic patients when substituted by alanine.

In the region of SEQ ID NO: 210 important for binding, the amino acids at positions 1, 2 and 6 were identified as amino acids particularly important for binding to IgE antibodies from allergic patients. The amino acids at positions 3-5 of SEQ ID NO: 210 had no influence on the binding activity against IgE antibodies from allergic patients when substituted by alanine.

Hereinafter, epitopes 2 to 55 were similarly identified, and the results were summarized below in Table 3.

TABLE 3-1

| | | Positions of | | Particularly | Binding- | Non-binding- |
|---|---|---|---|---|---|---|
| | | amino acids of | SEQ ID NO | important | influencing | influencing |
| | | region important | of region | amino acid in | amino acid in | amino acid in |
| Epitope | SEQ ID NO | for binding | important | region important | region important | region important |
| No. | of epitope | in epitope | for binding | for binding | for binding | for binding |
| 1 | 150 | 1-10 | 207 | 7-9 | 1, 10 | 2-6 |
| 1 | 150 | 9-14 | 210 | 1, 2, 6 | — | 3-5 |
| 2 | 151 | 1-10 | 213 | 8-10 | 1, 6, 7 | 2-5 |
| 2 | 151 | 1-10 | 213 | 7, 9, 10 | 1, 2 | 3-6, 8 |
| 2 | 151 | 9-15 | 218 | 1, 3, 5 | 4, 6, 7 | 2 |
| 3 | 152 | 1-15 | 152 | 1, 3-15 | — | 2 |
| 4 | 153 | 1-15 | 153 | 1, 3, 7, 9, | 4, 5, 8 | 2, 6, 10-13 |
| | | | | 14, 15 | | |
| 5 | 154 | 1-12 | 224 | 1, 3, 4, 10, | 2, 11 | 5-9 |
| | | | | 12 | | |
| 5 | 154 | 11-14 | 227 | 3, 4 | 1 | 2 |
| 6 | 155 | 1-11 | 230 | 3, 4, 6, 7, | 1, 2, 5, 9, | 8 |
| | | | | 11 | 10 | |
| 7 | 156 | 3-10 | 232 | 1, 8 | — | 2-7 |
| 7 | 156 | 1-10 | 235 | 1-3, 5, 8, 10 | 4, 9 | 6, 7 |
| 8 | 157 | 2-9 | 237 | 1, 3, 7, 8 | — | 2, 4-6 |
| 9 | 158 | 5-10 | 239 | 1, 2, 6 | — | 3-5 |
| 9 | 158 | 5-11 | 242 | 1, 2, 6, 7 | 3, 4 | 5 |
| 10 | 159 | 5-15 | 245 | 1, 5, 8, 11 | 3 | 2, 4, 6, 7, 9, |
| | | | | | | 10 |
| 10 | 159 | 1-11 | 247 | 1, 2, 11 | — | 3-10 |
| 11 | 160 | 9-15 | 250 | 1, 6, 7 | 2, 5 | 3, 4 |
| 12 | 161 | 4-6 | 252 | 1, 3 | — | 2 |
| 12 | 161 | 9-15 | 255 | 2, 6, 7 | 1, 3 | 4, 5 |
| 13 | 162 | 1-7 | 258 | 1, 3, 5, 7 | 2, 6 | 4 |
| 13 | 162 | 8-14 | 261 | 1, 5-7 | 2, 3 | 4 |
| 14 | 163 | 1-5 | 263 | 3-5 | — | 1, 2 |
| 14 | 163 | 1-9 | 266 | 1, 3, 5, 6 | 2, 4, 7, 9 | 8 |
| 15 | 164 | 8-15 | 269 | 2, 7 | 4, 6 | 1, 3, 5, 8 |
| 16 | 165 | 2-6 | 271 | 3, 5 | — | 1, 2, 4 |
| 16 | 165 | 8-15 | 273 | 1-4, 8 | — | 5-7 |
| 18 | 167 | 2-10 | 276 | 1, 3, 9 | 4, 7 | 2, 5, 6, 8 |
| 18 | 167 | 8-12 | 279 | 3-5 | 1 | 2 |
| 19 | 168 | 2-10 | 282 | 1-3, 8, 9 | 5, 7 | 4, 6 |
| 19 | 168 | 11-15 | 284 | 1, 2, 4, 5 | — | 3 |
| 20 | 169 | 1-11 | 287 | 1, 3, 7, 11 | 5 | 2, 4, 6, 8-10 |
| 20 | 169 | 7-14 | 290 | 1, 3, 5, 8 | 7 | 2, 4, 6 |
| 21 | 170 | 2-15 | 292 | 1, 5, 14 | — | 2-4, 6-13 |
| 22 | 171 | 4-13 | 295 | 2, 5, 8 | 1, 10 | 3, 4, 6, 7, 9 |
| 22 | 171 | 8-13 | 297 | 1, 4, 6 | — | 2, 3, 5 |
| 22 | 171 | 8-15 | 300 | 4, 5, 7, 8 | 1, 3 | 2, 6 |
| 24 | 173 | 2-7 | 303 | 1, 5 | 6 | 2-4 |
| 24 | 173 | 1-9 | 306 | 2, 4, 9 | 1, 3, 6-8 | 5 |
| 24 | 173 | 12-15 | 308 | 1, 3, 4 | — | 2 |
| 25 | 174 | 1-7 | 310 | 1, 3, 4, 7 | — | 2, 5, 6 |
| 26 | 175 | 5-10 | 312 | 1, 2, 6 | — | 3-5 |
| 26 | 175 | 11-15 | 314 | 1, 2, 5 | — | 3, 4 |
| 27 | 176 | 2-12 | 317 | 6, 8, 11 | 1, 9, 10 | 2-5, 7 |
| 28 | 177 | 1-9 | 320 | 2, 4, 5 | 1, 6, 8, 9 | 3, 7 |
| 28 | 177 | 7-15 | 323 | 2, 5, 8, 9 | 1, 7 | 3, 4, 6 |
| 29 | 178 | 3-14 | 326 | 1-5, 8 | 10, 12 | 6, 7, 9, 11 |
| 30 | 179 | 1-11 | 329 | 1, 6, 9-11 | 5, 7, 8 | 2-4 |
| 30 | 179 | 7-15 | 332 | 3, 6, 9 | 1, 2 | 4, 5, 7, 8 |
| 31 | 180 | 7-15 | 335 | 1, 4-6, 9 | 3, 7, 8 | 2 |
| 32 | 181 | 1-10 | 338 | 2, 4-6, 10 | 1 | 3, 7-9 |
| 33 | 182 | 2-9 | 341 | 3, 4, 6, 8 | 1 | 2, 5, 7 |
| 33 | 182 | 2-11 | 344 | 3, 7, 8, 10 | 1, 4, 6 | 2, 5, 9 |
| 33 | 182 | 7-15 | 347 | 1, 6, 8, 9 | 2 | 3-5, 7 |
| 33 | 182 | 9-14 | 350 | 1, 4, 6 | 5 | 2, 3 |
| 34 | 183 | 5-14 | 353 | 2, 6, 8, 10 | 9 | 1, 3-5, 7 |
| 35 | 184 | 5-12 | 356 | 1, 5, 8 | 7 | 2-4, 6 |
| 35 | 184 | 9-15 | 359 | 1, 4, 6 | 3, 7 | 2, 5 |
| 36 | 185 | 5-12 | 362 | 3, 6, 8 | 1, 7 | 2, 4, 5 |
| 36 | 185 | 7-15 | 365 | 4, 6, 8, 9 | 1, 5 | 2, 3, 7 |
| 37 | 186 | 1-10 | 367 | 1, 3, 10 | — | 2, 4-9 |
| 37 | 186 | 11-15 | 370 | 1, 2, 5 | 4 | 3 |
| 38 | 187 | 2-11 | 373 | 1, 3, 6-8 | 9, 10 | 2, 4, 5 |
| 38 | 187 | 7-14 | 376 | 1, 3-6 | 7, 8 | 2 |
| 39 | 188 | 1-10 | 379 | 1, 6, 10 | 4, 7, 8 | 2, 3, 5, 9 |
| 39 | 188 | 8-15 | 382 | 3, 5, 7, 8 | 1 | 2, 4, 6 |
| 40 | 189 | 2-8 | 384 | 1, 3, 4, 7 | — | 2, 5, 6 |
| 41 | 190 | 2-9 | 386 | 1, 2, 7, 8 | — | 3-6 |
| 41 | 190 | 1-10 | 388 | 1, 4, 8-10 | — | 2, 3, 5-7 |
| 41 | 190 | 9-15 | 390 | 1, 6, 7 | — | 2-5 |
| 42 | 191 | 6-11 | 392 | 1-4 | — | 5, 6 |
| 42 | 191 | 6-15 | 395 | 1, 3, 7, 10 | 8 | 2, 4-6, 9 |
| 43 | 192 | 5-8 | 397 | 1, 3 | — | 2, 4 |
| 43 | 192 | 7-15 | 400 | 1, 2, 5, 6 | 7, 8, 9 | 3, 4 |
| 44 | 193 | 1-7 | 402 | 1, 5-7 | — | 2-4 |
| 44 | 193 | 6-15 | 405 | 1-3, 6, 8, 10 | 5, 7 | 4, 9 |
| 45 | 194 | 3-7 | 407 | 1, 2, 3 | — | 4, 5 |
| 46 | 195 | 2-13 | 409 | 1, 9, 10, 12 | — | 2-8, 11 |
| 47 | 196 | 1-10 | 411 | 1, 7, 9, 10 | — | 2-6, 8 |
| 47 | 196 | 1-8 | 413 | 1, 4, 8 | — | 2, 3, 5-7 |
| 48 | 197 | 7-10 | 415 | 1, 3, 4 | — | 2 |
| 48 | 197 | 9-14 | 417 | 1, 6 | — | 2-5 |
| 49 | 198 | 5-13 | 420 | 2-5, 7, 8 | 1, 9 | 6 |
| 50 | 199 | 1-12 | 423 | 2, 4, 12 | 1, 6, 10 | 3, 5, 7-9, 11 |
| 50 | 199 | 9-15 | 425 | 1, 4, 7 | — | 2, 3, 5, 6 |
| 51 | 200 | 6-13 | 428 | 1-5 | 6, 8 | 7 |
| 52 | 201 | 1-10 | 431 | 1, 4, 6, 10 | 7, 9 | 2, 3, 5, 8 |
| 52 | 201 | 6-15 | 434 | 1, 5, 7, 8, | 6, 9 | 2-4 |
| | | | | 10 | | |
| 53 | 202 | 6-15 | 436 | 1, 2, 7-10 | — | 3-6 |
| 54 | 203 | 1-15 | 203 | 1, 9, 13-15 | 10, 11 | 2-8, 12 |
| 55 | 204 | 3-10 | 441 | 1, 2, 5, 8 | — | 3, 4, 6, 7 |
| 55 | 204 | 7-12 | 444 | 1, 2, 3, 6 | 4 | 5 |

### Example 11: Confirmation of Epitope Cross-Reactivity

Amino acids other than particularly important amino acids in the region (SEQ ID NO: 250) important for binding, in epitope No. 11 having the amino acid sequence “SMVLVKMKEIAEAYL” (SEQ ID NO: 160) found in salmon heat shock cognate 70 kDa protein in Table 3 were defined as any given amino acid (X). NCBI was searched for proteins having the resulting amino acid sequence “EXXXXYL” (SEQ ID NO: 248). As a result, the amino acid sequence “AMVLVKMKETAEAYL” (SEQ ID NO: 451) of bastard halibut heat shock 70 kDa protein (accession number: XP_019944736.1) was identified.

The binding activity of peptides comprising the amino acid sequences of SEQ ID NOs: 160 and 451 against an IgE antibody from one patient having allergies to salmons and bastard halibut, a peach and an apple was confirmed by ELISA. The peptides were synthesized such that the peptides were N-terminally biotinylated by the Fmoc method.

To be specific, ELISA was carried out according to the following procedure.
(1) The concentrations of the biotinylated peptides were adjusted to 1 μg/mL with PBST.
(2) Each peptide solution was added at 40 μL to each well of a 384-well plate coated with streptavidin, and shaken at room temperature for one hour. After collection of the solution, the wells were washed with PBS five times.
(3) A 1:5 dilution of Blocking one (diluted with MQ) was added at 40 μL thereto and shaken at room temperature for 15 minutes. After removal of the solution, the wells were washed with PBS five times.
(4) A diluted serum solution (1:10, diluting solution: Blocking one produced by Nacalai Tesque, Inc.) was added at 40 μL thereto and shaken at room temperature for one hour. After removal of the solution, the wells were washed with PBS five times.
(5) A diluted secondary antibody solution (1:5000, diluting solution: Blocking one produced by Nacalai Tesque, Inc.) was added at 40 μL thereto and shaken at room temperature for one hour. After removal of the solution, the wells were washed with PBS five times.
(6) 1-Step Ultra TMB-ELISA (Thermo Fisher Scientific) was added at 40 μL thereto and shaken at room temperature for 15 minutes.
(7) 2 M H₂SO₄ was added at 40 μL thereto. Absorbance at 450 nm was measured.

A peptide having this amino acid sequence “AMVLVKMKETAEAYL” of bastard halibut heat shock 70 kDa protein and a peptide having the amino acid sequence “SMVLVKMKEIAEAYL” of salmon heat shock cognate 70 kDa protein were each prepared by the same procedure as in Example 10(A), and the presence or absence of binding thereto of IgE antibodies present in the serum of a fish-allergic patient having allergy symptoms to salmon and bastard halibut and the serum of a non-fish-allergic subject (serum mixture from 4 subjects) was measured by the same technique as therein.

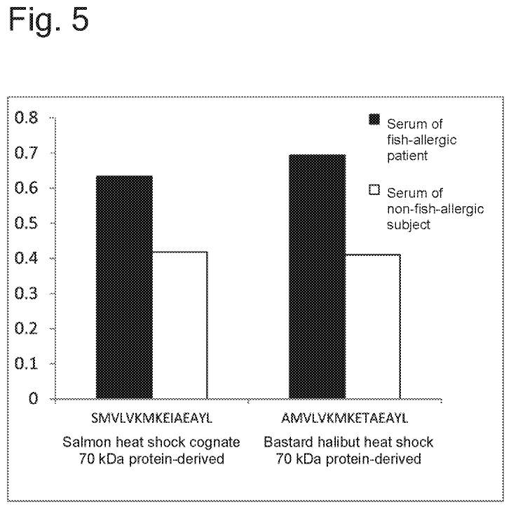

The results are shown in FIG. 5.

As is evident from FIG. 5, an IgE antibody bound in a patient-specific manner to the peptide having the amino acid sequence derived from bastard halibut heat shock 70 kDa protein, as in the peptide having the amino acid sequence derived from salmon heat shock cognate 70 kDa protein. Thus, epitope No. 11 was confirmed as an epitope exhibiting cross-reactivity between the fish species salmon and bastard halibut. In addition to epitope No. 11, epitope No. 1 to No. 55 (SEQ ID NOs: 150-204) identified in Example 10 were also confirmed to have cross-reactivity.

These epitopes have cross-reactivity with bastard halibut as well as horse mackerel, conger, blackhead seabream, mackerel, sea bream, cod, amberjack, eel, and the like. This indicates that these epitopes can be used for detecting antigen cross-reactivity irrespective of fish species.

1.-11. (canceled)

12. A method for detecting binding between an antigen and an antibody present in a sample from a fish-allergic patient, said method comprising contacting the antigen with said antibody to detect binding, wherein said antigen is a polypeptide comprising:
(a) an epitope having the amino acid sequence selected from SEQ ID NOs: 179-185 and 327-365, wherein (i) the polypeptide has an amino acid sequence having at least 90% sequence identity to the amino acid sequence of any one of SEQ ID NO: 70 or (ii) the polypeptide is encoded by a nucleotide sequence having at least 90% identity to the nucleotide sequence of SEQ ID NO: 69;
(b) an epitope having the amino acid sequence selected from SEQ ID NOs: 187-196 and 371-413, wherein (i) the polypeptide has an amino acid sequence having at least 90% sequence identity to the amino acid sequence of any one of SEQ ID NO: 121 or (ii) the polypeptide is encoded by a nucleotide sequence having at least 90% identity to the nucleotide sequence of SEQ ID NO: 120;
(c) an epitope having the amino acid sequence selected from SEQ ID NOs: 155 and 228-230, wherein (i) the polypeptide has an amino acid sequence having at least 90% sequence identity to the amino acid sequence of any one of SEQ ID NO: 5 or (ii) the polypeptide is encoded by a nucleotide sequence having at least 90% identity to the nucleotide sequence of SEQ ID NO: 4; or
(d) an epitope having the amino acid sequence selected from SEQ ID NOs: 168-171 and 280-300, wherein (i) the polypeptide has an amino acid sequence having at least 90% sequence identity to the amino acid sequence of any one of SEQ ID NO: 43 or (ii) the polypeptide is encoded by a nucleotide sequence having at least 90% identity to the nucleotide sequence of SEQ ID NO: 42;
wherein said polypeptide as defined in any one of (a), (b), (c) and (d) specifically binds to said antibody, and wherein said antigen is immobilized to a carrier or a surface.

13. The method according to claim 12, wherein the polypeptide is:
(a) a polypeptide comprising the epitope having the amino acid sequence selected from SEQ ID NOs: 179-185 and 327-365, wherein (i) the polypeptide has an amino acid sequence having at least 95% identity to the amino acid sequence of any one of SEQ ID NO: 70 or (ii) the polypeptide is encoded by a nucleotide sequence having at least 95% identity to the nucleotide sequence of SEQ ID NO: 69;
(b) a polypeptide comprising the epitope having the amino acid sequence selected from SEQ ID NOs: 187-196 and 371-413, wherein (i) the polypeptide has an amino acid sequence having at least 95% identity to the amino acid sequence of any one of SEQ ID NO: 121 or (ii) the polypeptide is encoded by a nucleotide sequence having at least 95% identity to the nucleotide sequence of SEQ ID NO: 120;
(c) a polypeptide comprising the epitope having the amino acid sequence selected from SEQ ID NOs: 155 and 228-230, wherein (i) the polypeptide has an amino acid sequence having at least 95% identity to the amino acid sequence of any one of SEQ ID NO: 5 or (ii) the polypeptide is encoded by a nucleotide sequence having at least 95% identity to the nucleotide sequence of SEQ ID NO: 4; or
(d) a polypeptide comprising the epitope having the amino acid sequence selected from SEQ ID NOs: 168-171 and 280-300, wherein (i) the polypeptide has an amino acid sequence having at least 95% identity to the amino acid sequence of any one of SEQ ID NO: 43 or (ii) the polypeptide is encoded by a nucleotide sequence having at least 95% identity to the nucleotide sequence of SEQ ID NO: 42.

14. A method for detecting binding between an antigen and an antibody present in a sample from a fish-allergic patient, said method comprising detecting binding between said antigen and said antibody with a reagent capable of detecting binding between the antigen and the antibody, wherein said antigen is a polypeptide comprising:
(a) an epitope having the amino acid sequence selected from SEQ ID NOs: 179-185 and 327-365, wherein (i) the polypeptide has an amino acid sequence having at least 90% sequence identity to the amino acid sequence of any one of SEQ ID NO: 70 or (ii) the polypeptide is encoded by a nucleotide sequence having at least 90% identity to the nucleotide sequence of SEQ ID NO: 69;
(b) an epitope having the amino acid sequence selected from SEQ ID NOs: 187-196 and 371-413, wherein (i) the polypeptide has an amino acid sequence having at least 90% sequence identity to the amino acid sequence of any one of SEQ ID NO: 121 or (ii) the polypeptide is encoded by a nucleotide sequence having at least 90% identity to the nucleotide sequence of SEQ ID NO: 120;
(c) an epitope having the amino acid sequence selected from SEQ ID NOs: 155 and 228-230, wherein (i) the polypeptide has an amino acid sequence having at least 90% sequence identity to the amino acid sequence of any one of SEQ ID NO: 5 or (ii) the polypeptide is encoded by a nucleotide sequence having at least 90% identity to the nucleotide sequence of SEQ ID NO: 4; or
(d) an epitope having the amino acid sequence selected from SEQ ID NOs: 168-171 and 280-300, wherein (i) the polypeptide has an amino acid sequence having at least 90% sequence identity to the amino acid sequence of any one of SEQ ID NO: 43 or (ii) the polypeptide is encoded by a nucleotide sequence having at least 90% identity to the nucleotide sequence of SEQ ID NO: 42;
wherein said polypeptide as defined in any one of (a), (b), (c) and (d) specifically binds to said antibody.

15. The method according to claim 14, wherein the polypeptide is:
(a) a polypeptide comprising the epitope having the amino acid sequence selected from SEQ ID NOs: 179-185 and 327-365, wherein (i) the polypeptide has an amino acid sequence having at least 95% identity to the amino acid sequence of any one of SEQ ID NO: 70 or (ii) the polypeptide is encoded by a nucleotide sequence having at least 95% identity to the nucleotide sequence of SEQ ID NO: 69;
(b) a polypeptide comprising the epitope having the amino acid sequence selected from SEQ ID NOs: 187-196 and 371-413, wherein (i) the polypeptide has an amino acid sequence having at least 95% identity to the amino acid sequence of any one of SEQ ID NO: 121 or (ii) the polypeptide is encoded by a nucleotide sequence having at least 95% identity to the nucleotide sequence of SEQ ID NO: 120;
(c) a polypeptide comprising the epitope having the amino acid sequence selected from SEQ ID NOs: 155 and 228-230, wherein (i) the polypeptide has an amino acid sequence having at least 95% identity to the amino acid sequence of any one of SEQ ID NO: 5 or (ii) the polypeptide is encoded by a nucleotide sequence having at least 95% identity to the nucleotide sequence of SEQ ID NO: 4; or
(d) a polypeptide comprising the epitope having the amino acid sequence selected from SEQ ID NOs: 168-171 and 280-300 wherein (i) the polypeptide has an amino acid sequence having at least 95% identity to the amino acid sequence of any one of SEQ ID NO: 43 or (ii) the polypeptide is encoded by a nucleotide sequence having at least 95% identity to the nucleotide sequence of SEQ ID NO: 42.


SEQUENCE LISTING (451 sequences)

SEQ ID NO: 1 - DNA, 2685 bp, synthetic construct
  misc_feature 1..2685: note = PREDICTED: alpha-actinin-3
  source 1..2685: mol_type = other DNA
atgacggcaatcgaaactcaggtgcaatatggctcctacatgacttcggagcaagtctacatgacccaagaggatgattgggacagggaccttctgttggaccctgcctgggagaaacagcagcgcaagaccttcaccgcctggtgcaactctcacctgcgtaaagcgggcacacagattgagaacattgaggaggacttcaggaatgggctcaagctcatgttgctgttagaggtcatctcaggtgaaaggcttcccaaaccagacaaaggcaaaatgcgtttccacaaaatcgccaacgtgaacaaggccctggatttcatctgcagcaagggagtcaagctggtgtccatcggtgccgaggagattgtggatggtaatgtgaagatgaccctggggatgatctggaccatcattctgcgtttcgccatccaggacatctctgtagaggagacctctgctaaggagggtctgttgctgtggtgccagaggaagactgccccctacaggaatgtcaatgtgcagaacttccacatcagttggaaggatggcctggcactgtgtgccctcatccacagacacagacctgacctcattgactactccaaactgcgcaaggatgaccccatgggcaatctcaacactgcctttgaggtggcagagaagtacctggacatccccaagatgttggatgcagaagatattgtgaacaccccgaagcccgacgagaaagccatcatgacctatgtgtcctgcttctatcatgccttcgctggagccgagcaggccgagacagctgccaataggatctgcaaggtgttggctgtcaaccaggagaacgagaagctcatggaggagtatgagaagctggccagtgagctgctggagtggatccgtcgcaccatcccctggctggagaaccgcgtggctgagcagaccatgcgcgccatgcagcagaagctggaggacttccgtgactaccgtcgcgtccacaagcctcccaaggtgcaggagaagtgtcagctggagattaacttcaacaccctgcagaccaagctgaggctgagcaacaggcccgccttcatgccctccgagggcaagatggtgtcggacattgccaatgcctggaagggtctggagcaggtagagaagggctatgaggagtggctgctcactgagatcagacgcctggagaggctggaccacctggctgagaagttcaagcagaagtcttccctgcatgagtcctggacctcaggtaaggtggagctgctgtccatgaaggactatgagtctgcctcactgatggagatccgtgccctgatgaggaagcacgaggcgtttgagagcgacctggctgcccaccaggacagagtggagcagattgctgccatcgcccaggagctcaatgagctggactatcatgatgccgtcaccatcaacgcccgctgccagggtatttgtgaccagtgggacaacctgggcaccttgacccagaagaggagagactcactggagcgtgtggagaagctgtgggagaccatcgaccagctgtacctggagtttgccaagagagcagcccccttcaacaactggatggacggagccatggaggacttacaggacatgttcattgtgcacagcattgaggagatccagagtctgatcacagcccatgaccagttcaaggccaccctgccagaggcagataaggagcgcgttgcaaccatgggaatccagaatgagatcgtgaagatcgctcagacctacggcatcaagctgtcaggagtcaacccctacaccaacctttccccccaggacatcaccgacaaatgggatgctgtgaaacacctggtgcccctcagggatcagatgctgcaggaggaggtggccaggcagcagtccaacgagaggctgaggcgccagttcgctgcccaggccaacatcatcggaccctggatccagaccaagatggaggagatcagccatgtgtctgtggacatcgccggctccctggaggaacagatgagcaacctgaagcagtacgagcagaacatcatcaactacaagtgcaacatcgacaagctggagggagaccaccaactcagccaggagtccctcatctttgacaacaagcacaccaactacaccatggagcatgtgcgtgtgggctgggagcagctcctcaccaccatcgcccgcaccatcaacgaggtggagaaccagatcctgacccgagatgccaagggcatcagccaggagcagctcaacgagttcagagcctccttcaaccactttgacaggaagagaaatggtatgatggacccagatgacttccgtgcctgtctcatctccatgggctacgatctgggtgaggtggagtttgcccgcatcatgacgctggtggattccaacaacacaggtgtggtgaccttccaggccttcatcgacttcatgacccgcgagacggccgagacagacaccgccgatcaggtcatggcctcattcaagatcctggcctctgacaagacatacatcacagtggaagaactgcgcagggagctgcccccagagcaggccgactactgcatcagccgcatgaccagttacatcggcagcggcgcacccccaggcgccctggactacatctccttctccagtgccctgtacggagagagcgactta

SEQ ID NO: 2 - protein, 895 aa, synthetic construct
  REGION 1..895: note = PREDICTED: alpha-actinin-3
MTAIETQVQYGSYMTSEQVYMTQEDDWDRDLLLDPAWEKQQRKTFTAWCNSHLRKAGTQIENIEEDFRNGLKLMLLLEVISGERLPKPDKGKMRFHKIANVNKALDFICSKGVKLVSIGAEEIVDGNVKMTLGMIWTIILRFAIQDISVEETSAKEGLLLWCQRKTAPYRNVNVQNFHISWKDGLALCALIHRHRPDLIDYSKLRKDDPMGNLNTAFEVAEKYLDIPKMLDAEDIVNTPKPDEKAIMTYVSCFYHAFAGAEQAETAANRICKVLAVNQENEKLMEEYEKLASELLEWIRRTIPWLENRVAEQTMRAMQQKLEDFRDYRRVHKPPKVQEKCQLEINFNTLQTKLRLSNRPAFMPSEGKMVSDIANAWKGLEQVEKGYEEWLLTEIRRLERLDHLAEKFKQKSSLHESWTSGKVELLSMKDYESASLMEIRALMRKHEAFESDLAAHQDRVEQIAAIAQELNELDYHDAVTINARCQGICDQWDNLGTLTQKRRDSLERVEKLWETIDQLYLEFAKRAAPFNNWMDGAMEDLQDMFIVHSIEEIQSLITAHDQFKATLPEADKERVATMGIQNEIVKIAQTYGIKLSGVNPYTNLSPQDITDKWDAVKHLVPLRDQMLQEEVARQQSNERLRRQFAAQANIIGPWIQTKMEEISHVSVDIAGSLEEQMSNLKQYEQNIINYKCNIDKLEGDHQLSQESLIFDNKHTNYTMEHVRVGWEQLLTTIARTINEVENQILTRDAKGISQEQLNEFRASFNHFDRKRNGMMDPDDFRACLISMGYDLGEVEFARIMTLVDSNNTGVVTFQAFIDFMTRETAETDTADQVMASFKILASDKTYITVEELRRELPPEQADYCISRMTSYIGSGAPPGALDYISFSSALYGESDL

SEQ ID NO: 3 - protein, 14 aa, synthetic construct
  REGION 1..14: note = PREDICTED: alpha-actinin-3 fragment
ERVATMGIQNEIVK

SEQ ID NO: 4 - DNA, 3591 bp, synthetic construct
  misc_feature 1..3591: note = EEF1A2 binding protein-like
  source 1..3591: mol_type = other DNA
atgtggaaaaagaaatcaaagctcacggaccagacggccactggccaagttgggatcaagaagagatcaaaagtccctggagttatgatcacgcagtatgtggagaaaataccagatgggaaaagccaccctgacttcacccgcaagcctatcgcgttgaccattcaggagggtaaattcgccttcttcaaagccctggttattggagatccagaaccaaccgtgacatggggcagaaataatggagatgtgtcagatacatcaaaatatgtgacaaaatatgaccctgctacacgtgagcacttatttgagatggccaatgtaaaaccagaacaagcagacacctacaaatgctttgcagctaatgagtttggaagagcagtggtcacagtggtcctcaatgttattgaagttgggttcaagaaagcacaagcggactcacaggtgcaaccagaggcagctgttgcagattttaaatctgtattgaagagaaaaagtaaaattcagcccaaaatggaaaagaaagaagatggagaaatagatccaagattttgggaactcttgataagtgctgacaagaaagactatgagagcctcatgttggagtttggagtcactgacttccgctttatgctgaagacactgaatgagattaagaaggaaagagaggaagagcaagcacagttcattgaaaacctagctaacctgaaacctattgaagttggacccgatggctgtgcaaccttttcaatagacatggatctcattgaacaaagcagcaggatcttcctctacaaggatggagtgatgattccatacagcaaggagttgggagatacaatcaaacacagcctaaagatagtgggccgaaagtatcagttcagcataagggatctgtttcctgatgatgctgggctctaccaggtggatgttgaggacgtaaatgtattctccaccgattttaagattcccatggtggacttcctggtcaagattcaggagtgtaaggccatggagagagaggatgctgtgtttgagtgtgtcctgtcacagcccttcggcaagatcatgtgggttggcaagaacttaccattggaggcaggggataaatatgatattgaggtttcagaagacaagctcatccacagactaatcattaaagacgttgctatggtggacaaaggcatctatgccgctgtggcaggaatcaaatcttgcaatgcctttcttgtggttgaagccgacaagggtgaacccggcaaaaagaaacaacgtaaaaccacaagggcaggaggagctggagttgacctgacggcgattgcccaagagcaggcagttaaaaacacggcagacagagaggtgctgaaggagaaggtgaaagcaatcaatgacgagagagcggctaatgccactgcagcacctgagacttcagctgaagctaaagctaaagttaaggtggtagaggcttcccaaacaggagctccaaaacagggaccggcagttaaagggtctgaccataaatccgtggataatgagggatacagcattaagagtggactctcagatgtctttgctctccgtggcaagaaaggtgaactggtttgtgagatgagccatgaagttgatggggcctggttcaaggatggagagaagttatccaccacagatggaatagccatagtgaaagatggaacgagacacacgatgaccattcatagcagtagtgaagacgacactggagtctatcacattgaagcggggggatttaaatcagaggcaaaagtcactgtgggagaattacctgccattgatgctgatgacctccacaagttttctaagcctgtgacaataaaagtgggccagaatgcatcctggaagatgccttatacaccacaggacaacttggaggtgaaatggtttaaggatggcaaagagttgaaggatggtggtggggtgaagttggtgaaggaggtcaagcacagccggctgctgctccgggagtgtctgcgttctgacgctggggagatcaagattcaactcaaaaacccctttggcactatagaggccacatctcgactgattgtccttgacaagcccggcccaccagaaggcccggtggaaatcttggagaccacctccactgtgattgagctgcagtggggcgctcctaaggacgacggtggctccccggtgactaactacatcattgagcgccagcagctgggacagaccgtgtggaagaagatgggtgatgtcgcagctgacaaaaccacctacagggacaggaatgtggtccatgggaaactgtacatctacaatatctacgcagtgaacccagaggggaccagtgatgcactgcagactgaggaaacaatggctggcgtattgatatttgctggccgacctggagcaccaaaagtggtcagcgcatcaaagacctgcatcaacctgaaatgggagcccccagaagatgacggaggaattaagattgatggctatcaactggaaaaacgcaaaaaggacacagctcagtggattgctttgaacccagtaactgagcctattgaagtgctggagtacgcagtgaaggatgttgttgagggggcggagtatgagttcagggtatcggccatcaacgtttctggggcgggagagttcagtctcccctctgtgatggtgactgcaaagaatcccaacatgaggcctacattcaaagatccagaggacttcatggtgatcagggcgggaaactctgtgagaatcaaagttctctatgaggctgagcctccaccggagatcacttggatgaaggacaatgagcctgtatccagttttatacagatcatcaacacagagggctgttcccagcttgtgatcccctcaacaaaacgctctgattcaggaaattacaccattgtggctaagaataaagttggagaggccagctttgacattgaggtcctagtcacagatgagccaaagcctcctggtgcagtggagctggagcagattgtccatggcaaagttattgtgtcctgggaggcctctccagaccaggagctggacaacaggctgtattacatggtggccgagcacgactccagcacacgcatgtggcacactgtggcagaccgcatctttgacaattcatacacagccaataacatcatgcctggaagggagtaccacttcaaaatctatgccaagaacgacatgggcatgtcagacccctccatgtcacccacctggggcatcaacagcaacagaattcctataaacacaaacacgccagtggaagtcagctttgagaaaccaccatctgtcctggtccctctgaagatgcactcgccaccaaaaggattccagatgtacatgacctgcgccatccggggatgccccacacccagcgtcacgtggcacctgaacaacgtctgcatcaatggtgacagcaactactacatcaccaactcgtatggcgtgtgctccatgtacatccttagggtcaggcccaaggacgccggagaatacaaagttgtcgcagttaactccttcggcaaggcagaatgctccactaaacttgttgttaaagac

SEQ ID NO: 5 - protein, 1197 aa, synthetic construct
  REGION 1..1197: note = EEF1A2 binding protein-like
MWKKKSKLTDQTATGQVGIKKRSKVPGVMITQYVEKIPDGKSHPDFTRKPIALTIQEGKFAFFKALVIGDPEPTVTWGRNNGDVSDTSKYVTKYDPATREHLFEMANVKPEQADTYKCFAANEFGRAVVTVVLNVIEVGFKKAQADSQVQPEAAVADFKSVLKRKSKIQPKMEKKEDGEIDPRFWELLISADKKDYESLMLEFGVTDFRFMLKTLNEIKKEREEEQAQFIENLANLKPIEVGPDGCATFSIDMDLIEQSSRIFLYKDGVMIPYSKELGDTIKHSLKIVGRKYQFSIRDLFPDDAGLYQVDVEDVNVFSTDFKIPMVDFLVKIQECKAMEREDAVFECVLSQPFGKIMWVGKNLPLEAGDKYDIEVSEDKLIHRLIIKDVAMVDKGIYAAVAGIKSCNAFLVVEADKGEPGKKKQRKTTRAGGAGVDLTAIAQEQAVKNTADREVLKEKVKAINDERAANATAAPETSAEAKAKVKVVEASQTGAPKQGPAVKGSDHKSVDNEGYSIKSGLSDVFALRGKKGELVCEMSHEVDGAWFKDGEKLSTTDGIAIVKDGTRHTMTIHSSSEDDTGVYHIEAGGFKSEAKVTVGELPAIDADDLHKFSKPVTIKVGQNASWKMPYTPQDNLEVKWFKDGKELKDGGGVKLVKEVKHSRLLLRECLRSDAGEIKIQLKNPFGTIEATSRLIVLDKPGPPEGPVEILETTSTVIELQWGAPKDDGGSPVTNYIIERQQLGQTVWKKMGDVAADKTTYRDRNVVHGKLYIYNIYAVNPEGTSDALQTEETMAGVLIFAGRPGAPKVVSASKTCINLKWEPPEDDGGIKIDGYQLEKRKKDTAQWIALNPVTEPIEVLEYAVKDVVEGAEYEFRVSAINVSGAGEFSLPSVMVTAKNPNMRPTFKDPEDFMVIRAGNSVRIKVLYEAEPPPEITWMKDNEPVSSFIQIINTEGCSQLVIPSTKRSDSGNYTIVAKNKVGEASFDIEVLVTDEPKPPGAVELEQIVHGKVIVSWEASPDQELDNRLYYMVAEHDSSTRMWHTVADRIFDNSYTANNIMPGREYHFKIYAKNDMGMSDPSMSPTWGINSNRIPINTNTPVEVSFEKPPSVLVPLKMHSPPKGFQMYMTCAIRGCPTPSVTWHLNNVCINGDSNYYITNSYGVCSMYILRVRPKDAGEYKVVAVNSFGKAECSTKLVVKD

SEQ ID NO: 6 - protein, 17 aa, synthetic construct
  REGION 1..17: note = EEF1A2 binding protein-like fragment
AQADSQVQPEAAVADFK

SEQ ID NO: 7 - protein, 10 aa, synthetic construct
  REGION 1..10: note = EEF1A2 binding protein-like fragment
GIYAAVAGIK

SEQ ID NO: 8 - protein, 21 aa, synthetic construct
  REGION 1..21: note = EEF1A2 binding protein-like fragment
TTRAGGAGVDLTAIAQEQAVK

SEQ ID NO: 9 - DNA, 2532 bp, synthetic construct
  misc_feature 1..2532: note = Alpha-1,4 glucan phosphorylase
  source 1..2532: mol_type = other DNA
atgtcaaaaccgttgtcagatcacgatagaaagaagcagatctccgtgagaggtcttgctggtgtggaaaatgtggcagaactgaaggtcgctttcaacaggcatctccattttacgctggtcaaggacagaaatgtggcatccaaacgggattactactttgctctcgccaacaccgtgcgtgaccacttggtgggcaggtggatcagaacccagcaatactactatgagaaagatcccaaacgtgtgtactacatctccctggagttctacatgggtcgcaccctgcagaacaccatggtgaacctggcgctggaaaacgcctgtgatgaggccatataccagctgggtctggacatggaggagctggaggacatggaggaggacgcaggcctgggaaacggtggtcttggacgtcttgccgcctgcttcctggactctatggcttctctgggtctggctgcgtatggctacggtatccgctatgagtttggcatctttaaccagaagatcgtcaatggatggcaggttgaggaggctgatgactggctgcgttacggcaacccctgggagaaggcccgacccgagtacatgcgccccgtcaagttctatggcagaaccgagcacaccccagagggtgtgaaatgggttgacactcaggtagtgttggctctgccatatgacacccctatccccgggtacagaaacaacattgtcaacaccatgagactgtggtctgcaaaggccccatgcgacttcaacctgaaagacttcaacgttggtgggtacattcaggctgtgttggacagaaacctatgcgagaacatttcccgtgtgctgtaccccaatgataacttctttgagggcaaggagctgcgtctgaagcaggagtactttgtggtggccgccacccttcaggacatcgtccgtcgtttcaaggcctctaagtttggctccagagagatcgtccgcacagacttcgcccagctgcccaacaaagttgccatccagctgaatgacactcaccctgccatggccattcctgagctgatgagggtactggttgatgaggagaagctggagtgggacaaggcctgggacgtgtgtgtccgtacctgtgcctacacaaaccacaccgtgctgcctgaggccctggagcgctggcccattgacctgttccatcacctgctgccccgtcacctggagattatctacgagatcaaccgtcgcttcctgcagtacgtcgcctcgaagttccctggcgacaacgaccgtctgcgccgcatgtccctgattgaggagggagaatgcaagaaagtcaacatggctcatatgtgtatcgttggatcccatgctgtcaacggcgtggcccgcatccactcggagatcctcgtcgccactctgttcaaggacttttatgagttggacccacacaagttccagaacaagaccaatggcatcaccccccgtcgctggctggttatgtgcaaccccggcttggctgaggtcatcgcagagagaattggagaggagtttgtccgtgaccttgaccagctgaagaaactgttgaagttcattgatgatgatgctttcatccgtgacatagccaaagtcaagcaggagaacaagctgaagttcgctgtgcacctggaagaacactacaaagtaaagatcaacccccagtccatgtttgacttccaagtcaaaagaatccacgagtacaagagacagctgctcaactgtctgcacatgatcacctactacaaccgtatcaagaaggagccaaacaagcactggaccccaagaaccatcatggtcggaggaaaggctgccccaggctaccacacagccaagatgatcattcgtctcatcaccgctatcggtgaggttgtcaaccacgaccccgtgatcggcgaccgcctcaaagtcatcttcttggagaactacagagtcaccctggctgagaaagccatcccctctgctgacctgtctgagcagatctctacagctggcactgaggcctctggcactggcaacatgaagttcatgctgaatggcgctctgaccatcggcaccatggacggagccaacgtggagatggccgaggaggccggagagaagaacctcttcatcttcggcatgagagtggaggaagtggacgcaatggacgccggcaaaggataccacgcctctgagtactacaaccgtattcccgagctgaaacaggccatggaccagatctctggtggcttcttcagccataagcagccagacctcttcaaggaacttgtggacctgctgatgcaccacgacaggttcaaggtgtttgctgactacgaagcctacatcaaaagtcaggataaggtcaacgaactgtacaagaaacccaaggaatggaccaagatggtgatccataacattgcaggctgtggtaaattctccagcgaccgcaccatttcccagtacgcccgggagatctggggcatggagcccagcctggagaagatccctgcccccgatgagcaactcaaa

SEQ ID NO: 10 - protein, 844 aa, synthetic construct
  REGION 1..844: note = Alpha-1,4 glucan phosphorylase
MSKPLSDHDRKKQISVRGLAGVENVAELKVAFNRHLHFTLVKDRNVASKRDYYFALANTVRDHLVGRWIRTQQYYYEKDPKRVYYISLEFYMGRTLQNTMVNLALENACDEAIYQLGLDMEELEDMEEDAGLGNGGLGRLAACFLDSMASLGLAAYGYGIRYEFGIFNQKIVNGWQVEEADDWLRYGNPWEKARPEYMRPVKFYGRTEHTPEGVKWVDTQVVLALPYDTPIPGYRNNIVNTMRLWSAKAPCDFNLKDFNVGGYIQAVLDRNLCENISRVLYPNDNFFEGKELRLKQEYFVVAATLQDIVRRFKASKFGSREIVRTDFAQLPNKVAIQLNDTHPAMAIPELMRVLVDEEKLEWDKAWDVCVRTCAYTNHTVLPEALERWPIDLFHHLLPRHLEIIYEINRRFLQYVASKFPGDNDRLRRMSLIEEGECKKVNMAHMCIVGSHAVNGVARIHSEILVATLFKDFYELDPHKFQNKTNGITPRRWLVMCNPGLAEVIAERIGEEFVRDLDQLKKLLKFIDDDAFIRDIAKVKQENKLKFAVHLEEHYKVKINPQSMFDFQVKRIHEYKRQLLNCLHMITYYNRIKKEPNKHWTPRTIMVGGKAAPGYHTAKMIIRLITAIGEVVNHDPVIGDRLKVIFLENYRVTLAEKAIPSADLSEQISTAGTEASGTGNMKFMLNGALTIGTMDGANVEMAEEAGEKNLFIFGMRVEEVDAMDAGKGYHASEYYNRIPELKQAMDQISGGFFSHKQPDLFKELVDLLMHHDRFKVFADYEAYIKSQDKVNELYKKPKEWTKMVIHNIAGCGKFSSDRTISQYAREIWGMEPSLEKIPAPDEQLK

SEQ ID NO: 11 - protein, 9 aa, synthetic construct
  REGION 1..9: note = Alpha-1,4 glucan phosphorylase fragment
IPAPDEQLK

SEQ ID NO: 12 - protein, 17 aa, synthetic construct
  REGION 1..17: note = Alpha-1,4 glucan phosphorylase fragment
QISVRGLAGVENVAELK

SEQ ID NO: 13 - protein, 25 aa, synthetic construct
  REGION 1..25: note = Alpha-1,4 glucan phosphorylase fragment
AIPSADLSEQISTAGTEASGTGNMK

SEQ ID NO: 14 - protein, 9 aa, synthetic construct
  REGION 1..9: note = Alpha-1,4 glucan phosphorylase fragment
DFYELDPHK

SEQ ID NO: 15 - protein, 10 aa, synthetic construct
  REGION 1..10: note = Alpha-1,4 glucan phosphorylase fragment
FAVHLEEHYK

SEQ ID NO: 16 - protein, 15 aa, synthetic construct
  REGION 1..15: note = Alpha-1,4 glucan phosphorylase fragment
GYHASEYYNRIPELK

SEQ ID NO: 17 - protein, 9 aa, synthetic construct
  REGION 1..9: note = Alpha-1,4 glucan phosphorylase fragment
IPAPDEQLK

SEQ ID NO: 18 - DNA, 2574 bp, synthetic construct
  misc_feature 1..2574: note = Elongation factor 2
  source 1..2574: mol_type = other DNA
atggtgaactttacagtggaccagatccgtgccatcatggacaagaaatccaacattcgtaacatgtctgtgatcgctcacgtggaccacggcaagtccacgctgaccgactccctggtgtctaaagccgggatcatcgcggggtctcgcgccggagagacacgcttcacagacactcgcaaagacgagcaggagcgctgtattaccatcaagtccacggctatctcgatgtactatgagctgggggaaaacgacatggccttcatcaagcagtctaaggatgggcttggcttcctcatcaacctgattgactcaccgggccatgtggacttctcctctgaggtcacagccgcccttagggtcaccgacggcgccctggtggtggttgactgcgtctcaggtgtgtgtgtgcagacagagaccgtgttgaggcaggccattgctgagcgcatcaagccagtgctgatgatgaacaagatggaccgggccctgctggagctgcagctggagcctgaggacctgttccagaccttccagcgcatcgtggagaatgtcaacgtcatcattgccacctacggagaggatgaagcgggaccaatgggtgccatcatgattgaccctgtgattggtaccgtggggtttgggtctggcctccacggctgggccttcactctaaagcagtttgctgagatgtacgtgacaaagttttctgccggcaaagacacccagctgggatcggcggagaggtgtaagaaggtggaggacatgatgaagaagctgtggggggagaggttttttgacccagccactgggaagttcagtaagtccaacctcggccctgacggtaagaagctgccccgcaccttctctcagctggtcctggaccctatcttcaaggtatttgatgccatcatgaacttcaagaaggatgagacagccaagctgatagagaagctggacatcaagctggactctgaggacaaggagaaggagggcaagcccctgttgaaggcagtgatgcgtcgctggctcccagccggagaagccctgctccagatgatcaccatccacctgccctcccccgtcacggcccagaagtaccgctgtgagctgctctacgagggaccaggagacgacgaggccgccatgggtatcaagaactgcgaccccaaggctcccctgatgatgtacatatctaagatggtgcccaccacagacaagggtcgcttctatgcctttggccgtgtgttctctggctgtgtgtccaccggtctgaaggtgcgcatcatgggaccaaacttcacccctgggaagaaggaagacctctacatcaagcccatccagaggaccattctgatgatggggcgttatgtggagcccattgaggatgtaccatgtgggaacatcgttgggctggttggagttgaccagtatctgattaagactgggaccatcaccacctttgaacaggcccacaacatgcgtgtgatgaagttcagcgtcagccctgtggtgagggtggctgtggaggccaagaaccctgctgacctgcccaagctggtggaggggctgaagcgtctggccaagtctgaccccatggtgcagtgtatcatcgaggagtctggagagcacatcatcgccggggccggagagcttcatctggagatctgtctcaaggatctggaggaggaccacgccggcattcccctgaagaaatctgatccagtggtgtcctacagggagactgtgtctgaggagtctgaagtgatgtgtctatccaagtcccctaacaagcacaaccgtctgtacatgcgggctaaacctttccctgacggcctggccgaggacatcgagaagggggacgtcagcccccgacaggagctgaaaatccgcgcccgtttcctggctgacaagtacgagtgggacgtgtcggaggcccgtaagatctggtgcttcggccctgacggtaccggtcccaacctgctgatggatgttaccaagggagtccagtacctgaatgagatcaaggacagtgttgtggctggcttccagtgggctgtcaaggagggtgtgttgtgtgaggagaacatgcgtgcagtccgcttcgacatccacgacgtgaccctgcacacagacgcaattcaccgcggtggcggacagatcatccccacggcccgcagagtgctgtatgcctgccagctcaccgcccagccacgactcatggagccggtctacttagtggagatccagtgcccagagcaggtagtgggtgggatctacggcgtgctgaacaggaagcgaggccatgtgttcgaggagtcccaggtgatgggcacgcccatgttcatcgtcaaggcctacctgcctgtcaacgagtcatttgggttcaccgctgacctgcgctccaacacgggcggccaggctttcccccagtgtgtgtttgatcactggcagatcctccagggagatccccaggaccccaccaccaagaccgccattgtggtggccgagaccaggaaacgcaaggggctgaaagagggcatcccggccctggacaactacctggacaaattg

SEQ ID NO: 19 - protein, 858 aa, synthetic construct
  REGION 1..858: note = Elongation factor 2
MVNFTVDQIRAIMDKKSNIRNMSVIAHVDHGKSTLTDSLVSKAGIIAGSRAGETRFTDTRKDEQERCITIKSTAISMYYELGENDMAFIKQSKDGLGFLINLIDSPGHVDFSSEVTAALRVTDGALVVVDCVSGVCVQTETVLRQAIAERIKPVLMMNKMDRALLELQLEPEDLFQTFQRIVENVNVIIATYGEDEAGPMGAIMIDPVIGTVGFGSGLHGWAFTLKQFAEMYVTKFSAGKDTQLGSAERCKKVEDMMKKLWGERFFDPATGKFSKSNLGPDGKKLPRTFSQLVLDPIFKVFDAIMNFKKDETAKLIEKLDIKLDSEDKEKEGKPLLKAVMRRWLPAGEALLQMITIHLPSPVTAQKYRCELLYEGPGDDEAAMGIKNCDPKAPLMMYISKMVPTTDKGRFYAFGRVFSGCVSTGLKVRIMGPNFTPGKKEDLYIKPIQRTILMMGRYVEPIEDVPCGNIVGLVGVDQYLIKTGTITTFEQAHNMRVMKFSVSPVVRVAVEAKNPADLPKLVEGLKRLAKSDPMVQCIIEESGEHIIAGAGELHLEICLKDLEEDHAGIPLKKSDPVVSYRETVSEESEVMCLSKSPNKHNRLYMRAKPFPDGLAEDIEKGDVSPRQELKIRARFLADKYEWDVSEARKIWCFGPDGTGPNLLMDVTKGVQYLNEIKDSVVAGFQWAVKEGVLCEENMRAVRFDIHDVTLHTDAIHRGGGQIIPTARRVLYACQLTAQPRLMEPVYLVEIQCPEQVVGGIYGVLNRKRGHVFEESQVMGTPMFIVKAYLPVNESFGFTADLRSNTGGQAFPQCVFDHWQILQGDPQDPTTKTAIVVAETRKRKGLKEGIPALDNYLDKL

SEQ ID NO: 20 - protein, 10 aa, synthetic construct
  REGION 1..10: note = Elongation factor 2 fragment
TAIVVAETRK

SEQ ID NO: 21 - protein, 12 aa, synthetic construct
  REGION 1..12: note = Elongation factor 2 fragment
DLEEDHAGIPLK

SEQ ID NO: 22 - protein, 13 aa, synthetic construct
  REGION 1..13: note = Elongation factor 2 fragment
DLEEDHAGIPLKK

SEQ ID NO: 23 - protein, 12 aa, synthetic construct
  REGION 1..12: note = Elongation factor 2 fragment
DSVVAGFQWAVK

SEQ ID NO: 24 - protein, 10 aa, synthetic construct
  REGION 1..10: note = Elongation factor 2 fragment
GDVSPRQELK

SEQ ID NO: 25 - DNA, 1989 bp, synthetic construct
  misc_feature 1..1989: note = Heat shock cognate 70 kDa protein
  source 1..1989: mol_type = other DNA
atgtcaaagggaccagcagttggcattgacctgggcaccacctactcctgtgtgggtgtgtttcagcatggcaaagtggagatcatagccaacgaccagggaaacaggaccacacccagttatgtcgccttcacagacacagaaaggctgattggggacgcagccaagaaccaagtggccatgaaccccacaaacacagtgtttgatgctaagcggctgatagggcggaagtttgacgacagtgtcgtccaggcagacatgaaacactggccgtttacagtgatcaacgactcgacacggcccaaggtccaagtggagtacaagggagagaccaaggccttctacccagaggagatctcctccatggtgctggtcaagatgaaggagattgcagaggcctacctgggcaagactataaccaatgcagtggtcactgtgccagcttacttcaacgactctcagcgccaggccaccaaagatgcagggactatctcaggactcaacgtactccgcatcatcaatgagccaactgctgctgctatcgcctatggcctggacaagaaggtgggagtggagagaaacgtcctaatctttgacctaggcggaggtacgtttgatgtgtctatcctgaccatcgaagacgggatctttgaggtgaagtccacggccggagacacccatcttggaggagaggacttcgacaaccgcatggtcaaccacttcatctctgagttcaagcgcaaatacaagaaggacatcagcgataacaagagggccgtgcgtcgtctgcgcaccgcctgtgaacgtgccaagcgcaccctgtcctccagtacccaggccagcattgagattgactctctgtatgagggcgtcgacttctacacctccatcaccagggctcgctttgaggagctgaacgctgacctgttcagaggcaccctggaccccgtggagaagtctctgagggacgccaagatggacaaggcccaggttcacgacatcgtcctagtgggaggctccacacgcatccccaagatccaaaagttgctgcaggacttcttcaatgggaaggagctcaacaagagcatcaaccctgatgaggcggttgcctatggtgcagctgttcaggcggccatcttgtccggagacaagtctgagaacgtgcaggacctgctgctgctggacgtcacgccactgtccctgggcattgagacggccggaggggtcatgaccgtgctcatcaagaggaacaccaccatccccaccaagcagacgcagaccttcacaacatactcagacaaccagcctggggtgctcattcaggtatatgagggggaaagagccatgaccaaagacaacaacctactggggaagtttgagttgtgtgggattccaccagccccccggggtgtgcctcagatcgaggtcacctttgacattgacgccaacggcatcatgaacgtgtctgccgctgacaagagcaccggcaaggagaataagatcaccatcaccaatgacaaaggtcgtctgagtaaggaggacatagagcgcatggtccaggaggctgaacagtacaaagctgcggacgatgtccagagggacaaggtggcgtccaagaatggcctggagtcatacgccttcaacatgaagtccaccgtggaggacgagaagctcaagggcaagctcagcgacgaggacaaacagaagatcctggacaaatgcaatgaggtcatcagctggctggacaagaaccagtctgcagagaaggaggagtttgagcaccaccagaaggagctggagaaggtctgtaaccccatcatcaccaagctgtaccagggtgctggggggatgcctggagggatgcctggagggatgcctggagggatgcctggagggatgcctggggggatgcctggagggatgcccggggctggtggtgccgcacccggaggaggtggatcctctggaccaacaattgaggaagtcgac

SEQ ID NO: 26 - protein, 663 aa, synthetic construct
  REGION 1..663: note = Heat shock cognate 70 kDa protein
MSKGPAVGIDLGTTYSCVGVFQHGKVEIIANDQGNRTTPSYVAFTDTERLIGDAAKNQVAMNPTNTVFDAKRLIGRKFDDSVVQADMKHWPFTVINDSTRPKVQVEYKGETKAFYPEEISSMVLVKMKEIAEAYLGKTITNAVVTVPAYFNDSQRQATKDAGTISGLNVLRIINEPTAAAIAYGLDKKVGVERNVLIFDLGGGTFDVSILTIEDGIFEVKSTAGDTHLGGEDFDNRMVNHFISEFKRKYKKDISDNKRAVRRLRTACERAKRTLSSSTQASIEIDSLYEGVDFYTSITRARFEELNADLFRGTLDPVEKSLRDAKMDKAQVHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELCGIPPAPRGVPQIEVTFDIDANGIMNVSAADKSTGKENKITITNDKGRLSKEDIERMVQEAEQYKAADDVQRDKVASKNGLESYAFNMKSTVEDEKLKGKLSDEDKQKILDKCNEVISWLDKNQSAEKEEFEHHQKELEKVCNPIITKLYQGAGGMPGGMPGGMPGGMPGGMPGGMPGGMPGAGGAAPGGGGSSGPTIEEVD

SEQ ID NO: 27 - protein, 14 aa, synthetic construct
  REGION 1..14: note = Heat shock cognate 70 kDa protein fragment
AFYPEEISSMVLVK

SEQ ID NO: 28 - protein, 14 aa, synthetic construct
  REGION 1..14: note = Heat shock cognate 70 kDa protein fragment
EDIERMVQEAEQYK

SEQ ID NO: 29 - protein, 14 aa, synthetic construct
  REGION 1..14: note = Heat shock cognate 70 kDa protein fragment
HWPFTVINDSTRPK

SEQ ID NO: 30 - protein, 14 aa, synthetic construct
  REGION 1..14: note = Heat shock cognate 70 kDa protein fragment
NQSAEKEEFEHHQK

SEQ ID NO: 31 - protein, 22 aa, synthetic construct
  REGION 1..22: note = Heat shock cognate 70 kDa protein fragment
TITNAVVTVPAYFNDSQRQATK

SEQ ID NO: 32 - DNA, 2061 bp, synthetic construct
  misc_feature 1..2061: note = Serotransferrin
  source 1..2061: mol_type = other DNA
atgaaactgcttctcgtctcagcgctgctggggtgcttcgctacggtgtatgctgccccagctgagggaatggttagatggtgcgtaaagtcggaaaaagagctgaagaaatgccacgatctcgcagccaatgtggcggggttttcatgcgtgaggagggacgactctcttgaatgcatccaagccatcaagagggaagaggctgatgctatcactctggatggaggagatatttacatagctggcctccacaactacaacctgcagcccatcattgcagaggactatggtgaggactctgacacctgctattatgctgtggccgtggccaaaaagggcactgaatttgggttcctcgacctccgtggcaagaagtcctgccacaccgggttgggcaaatctgcaggctggaacattcccatcggtaccctggtgactgtgggccagatccaatgggccggcatcgaggacagacctgtggagtcggcggtgagcgacttcttcaatgccagctgtgctccaggagccaatagggattctcaactgtgccagttgtgtatgggagactgctccagatctcacaacgagccctactatgactattccggggccttccagtgcctgaaggatggagcaggagaggttgccttcatcaagcacctgactgtacctgccgcagagaaggcaagctatgagttgctgtgcaaggataacaccagagctcccatcgacagctacaagacctgccacctggccagagtacccgcccacgctgtggtcagccgcaaggaccccaggctggccaatctcatctacagcaagctgatggccgtcacgaacttcaacctgttctcctccgatggttatgctgccaagaacctgatgttcaaggactccactcagaatctagtacagctgccaatgaccaccgactccttcctctacctgggagctgagtacatgagcactatacgctccctgacaaaagcgcaggccacaggtgtcacctccagggccatcaaatggtgtgccgtgggccataaggagaaggtcaagtgtgacgcctggacaatcaacagcttcacagatggtgactccaggatcgaatgccaggacgcacccacagtggatgaatgcatcaagaagatcatgcgtaaagaggcagatgccatagcggtggatggtggggaggtgttcactgctggaaaatgtggtctggtccctgtcatggtggagcagtatgatgaagttcggtgcagcgcccctggtgaggcgtcatcctactttgcggtggcggtggcaaagaggggatctgggttgacctggacaaccctgaagggcaagaggtcatgccacaccggcttgggcaggaccgcaggctggaacatacccatgggtcttatccataggaggactatgaactgcgacttcaccacatacttcagtaagggctgtgctcctggatttgaggtggactctcccttctgtgcccagtgtaagggcagtgggaagtccgtgggaggagatgggtccaagtgcaaagccagctctgaagagcagtactacggctataacggagcattcagatgtctggttgaagatgctggagatgttgccttcattaaacacactattgtaccagagatgactgatggtagtggtccagtttgggcacagaacctgatgtcctctgactttgaactactctgccaggatggtactaccaagccagtcacacatttccgtgagtgccacctggccaaggtgcccgcccatgctgtgataacacgcccagagtcccgtggagaggttgtgtccatccttctggagcagcaggccaggttcggctcaagcggcagcgattcctcatttaatatgttcaagccagattttggaaagaacttgctcttcaaggattccacaaagtgtctccaggagatcccaagcggcaccaaattccagggtttccttggggaagagtacatgatcgccatgcaatcgctcagggagtgctccaacagtacctcagatttggagaaggcatgcactttccattcctgc

SEQ ID NO: 33 - protein, 687 aa, synthetic construct
  REGION 1..687: note = Serotransferrin
MKLLLVSALLGCFATVYAAPAEGMVRWCVKSEKELKKCHDLAANVAGFSCVRRDDSLECIQAIKREEADAITLDGGDIYIAGLHNYNLQPIIAEDYGEDSDTCYYAVAVAKKGTEFGFLDLRGKKSCHTGLGKSAGWNIPIGTLVTVGQIQWAGIEDRPVESAVSDFFNASCAPGANRDSQLCQLCMGDCSRSHNEPYYDYSGAFQCLKDGAGEVAFIKHLTVPAAEKASYELLCKDNTRAPIDSYKTCHLARVPAHAVVSRKDPRLANLIYSKLMAVTNFNLFSSDGYAAKNLMFKDSTQNLVQLPMTTDSFLYLGAEYMSTIRSLTKAQATGVTSRAIKWCAVGHKEKVKCDAWTINSFTDGDSRIECQDAPTVDECIKKIMRKEADAIAVDGGEVFTAGKCGLVPVMVEQYDEVRCSAPGEASSYFAVAVAKRGSGLTWTTLKGKRSCHTGLGRTAGWNIPMGLIHRRTMNCDFTTYFSKGCAPGFEVDSPFCAQCKGSGKSVGGDGSKCKASSEEQYYGYNGAFRCLVEDAGDVAFIKHTIVPEMTDGSGPVWAQNLMSSDFELLCQDGTTKPVTHFRECHLAKVPAHAVITRPESRGEVVSILLEQQARFGSSGSDSSFNMFKPDFGKNLLFKDSTKCLQEIPSGTKFQGFLGEEYMIAMQSLRECSNSTSDLEKACTFHSC

SEQ ID NO: 34 - protein, 12 aa, synthetic construct
  REGION 1..12: note = Serotransferrin fragment
AQATGVTSRAIK

SEQ ID NO: 35 - protein, 10 aa, synthetic construct
  REGION 1..10: note = Serotransferrin fragment
ATGVTSRAIK

SEQ ID NO: 36 - protein, 11 aa, synthetic construct
  REGION 1..11: note = Serotransferrin fragment
DPRLANLIYSK

SEQ ID NO: 37 - protein, 12 aa, synthetic construct
  REGION 1..12: note = Serotransferrin fragment
GTEFGFLDLRGK

SEQ ID NO: 38 - protein, 13 aa, synthetic construct
  REGION 1..13: note = Serotransferrin fragment
KGTEFGFLDLRGK

SEQ ID NO: 39 - protein, 14 aa, synthetic construct
  REGION 1..14: note = Serotransferrin fragment
KGTEFGFLDLRGKK

SEQ ID NO: 40 - protein, 18 aa, synthetic construct
  REGION 1..18: note = Serotransferrin fragment
LMAVTNFNLFSSDGYAAK

SEQ ID NO: 41 - protein, 11 aa, synthetic construct
  REGION 1..11: note = Serotransferrin fragment
RGSGLTWTTLK

SEQ ID NO: 42 - DNA, 1518 bp, synthetic construct
  misc_feature 1..1518: note = Myosin binding protein H-like
  source 1..1518: mol_type = other DNA
atgcctgcgaagcctgcccctatcaagaaggcagccccgaaagagcctgctcctaagaaggaggaagcacctgcacctgcacccgcggaggcggctcctgctgaagttgctccccctgccgaggtcgaagctccaccggcagaggctgcccctgccgaagcagctgcccctgctgaggcagctccaaccgaagaaaaggctcccgcagaacccgcgcctgagcagcccaaagcacccacaccccctcctccagatgagcccaccagtgagcccctggaagtgacagtggacgacaagagcgacacttcagtgaccatcacctggaggcctccaaagactattccagccaactctggcctggatggatacactgtggaaatcaccaaggagggaactgaagactggaaagcagccaacgaggatctgaccgtgtcctgccgttatgtcatcaagaaccagactaccggcgaccgtctgctgatccgagtggttgctgtaaaccctggtggccgcagcccccctgctaccatcgccgaccccgttctggtcaaggaggttggggctcgccccaaagtccgcctccctcgtttcctgaggcagaggtatgtcaagaaagtcggcgagaagatcaacattgtcatcccgtactctggcaaacctaagccagtggttagctggttaaaggatggacagcccctcgactcaaagagggccaacatccgtacatctgacagagacagcatcctgttcatccgtcaagcagagagggtggactctggctcatacgaaatgtgcgtgaaggtggacgactttgaggacaaagctgctatcatcatccagattattgagttacccgggccaccagccagcattaagattgtggatgtctggggattcaacgttgctttggagtggaccgtacccaaagataatggaaacacagagatcacaggctacactgtccagaaagctgacaagaagaccggggactggttcaacattttggagcactacgccagactaaatgccaccatctcagacctcattatgggcaacacctacaccttcagggtctttgcagagaacaagtgcggactcagtgaggaatgtgccgtgaccaagggagaagccaccattgtgaaggaggttattgactacaaacctaccccgttcgtggagcatgacttcaccgaggctcccaagttcaccactgttctgaacgacaggtccaccactgttggctacagcaccaagctgctgtgttctgtgaggggatgccccaagcctaagattatgtggatgaagaacaagatgattctcaacaacatggacaaccccaagtacaggatgatcagcacagggggcatctgcaccctggagatccgtaagcctggcccatacgacggtggtgagtatgtctgcagagccgagaatacactggggaaggtcgacaccggctgcaagctggaggtcagaaagcctgggcaagcagatgctgacaaagacaagaaggaacag

SEQ ID NO: 43 - protein, 506 aa, synthetic construct
  REGION 1..506: note = Myosin binding protein H-like
MPAKPAPIKKAAPKEPAPKKEEAPAPAPAEAAPAEVAPPAEVEAPPAEAAPAEAAAPAEAAPTEEKAPAEPAPEQPKAPTPPPPDEPTSEPLEVTVDDKSDTSVTITWRPPKTIPANSGLDGYTVEITKEGTEDWKAANEDLTVSCRYVIKNQTTGDRLLIRVVAVNPGGRSPPATIADPVLVKEVGARPKVRLPRFLRQRYVKKVGEKINIVIPYSGKPKPVVSWLKDGQPLDSKRANIRTSDRDSILFIRQAERVDSGSYEMCVKVDDFEDKAAIIIQIIELPGPPASIKIVDVWGFNVALEWTVPKDNGNTEITGYTVQKADKKTGDWFNILEHYARLNATISDLIMGNTYTFRVFAENKCGLSEECAVTKGEATIVKEVIDYKPTPFVEHDFTEAPKFTTVLNDRSTTVGYSTKLLCSVRGCPKPKIMWMKNKMILNNMDNPKYRMISTGGICTLEIRKPGPYDGGEYVCRAENTLGKVDTGCKLEVRKPGQADADKDKKEQ

SEQ ID NO: 44 - protein, 18 aa, synthetic construct
  REGION 1..18: note = Myosin binding protein H-like fragment
AAIIIQIIELPGPPASIK

SEQ ID NO: 45 - protein, 18 aa, synthetic construct
  REGION 1..18: note = Myosin binding protein H-like fragment
AAPAEAAAPAEAAPTEEK

SEQ ID NO: 46 - protein, 11 aa, synthetic construct
  REGION 1..11: note = Myosin binding protein H-like fragment
APAEPAPEQPK

SEQ ID NO: 47 - protein, 14 aa, synthetic construct
  REGION 1..14: note = Myosin binding protein H-like fragment
DNGNTEITGYTVQK

SEQ ID NO: 48 - protein, 20 aa, synthetic construct
  REGION 1..20: note = Myosin binding protein H-like fragment
EVIDYKPTPFVEHDFTEAPK

SEQ ID NO: 49 - protein, 17 aa, synthetic construct
  REGION 1..17: note = Myosin binding protein H-like fragment
FTTVLNDRSTTVGYSTK

SEQ ID NO: 50 - protein, 10 aa, synthetic construct
  REGION 1..10: note = Myosin binding protein H-like fragment
INIVIPYSGK

SEQ ID NO: 51 - protein, 19 aa, synthetic construct
  REGION 1..19: note = Myosin binding protein H-like fragment
INIVIPYSGKPKPVVSWLK

SEQ ID NO: 52 - protein, 33 aa, synthetic construct
  REGION 1..33: note = Myosin binding protein H-like fragment
NQTTGDRLLIRVVAVNPGGRSPPATIADPVLVK

SEQ ID NO: 53 - protein, 13 aa, synthetic construct
  REGION 1..13: note = Myosin binding protein H-like fragment
SDTSVTITWRPPK

SEQ ID NO: 54 - protein, 17 aa, synthetic construct
  REGION 1..17: note = Myosin binding protein H-like fragment
TIPANSGLDGYTVEITK

SEQ ID NO: 55 - DNA, 1377 bp, synthetic construct
  misc_feature 1..1377: note = Desmin (fragment)
  source 1..1377: mol_type = other DNA
atttcctcttaccgccgcactttcggttctggtattggatccaccccaggcatgtcctccatgttctcccacggtggacgcggctcctccggctcggcccacatgtcctccagagtctacgagatgaccaaaagctccgcccgccccagctactcctccggcagtattcgctcctcctccggcggcgcgatgcgctcgtacgccgggatgggcgagaagctggacttcaacctggccgacgccactaaccgcgacttcctggacaccagaaccaatgagaaggcagagttgcagcacctgaacgaccggttcgccagctacatcgagaaggtccgtttcctggagcagcagaacgctactctggtggtggagatcgagaggctgaggggtcacgagccgacccgcgtggccgagatgtacgaggaggagatgagagagctgaggcgtcaggtggacggcatgtccaatgaccgagcccgcatggaggtggagagagacaacttggctgacgacctgcagaaactcaaactcagactgcaggaggtgatccaccagagggaagaggcagagaacaacctgtctgccttcagagctgacgtggactctgccacgctggccaggctggacctggagagacgcattgaaagcctgcaggaggagatcaccttcctcaagaagatccacgaggaggagatccatgagctgacgagccagatgcaggagacctcggtgcaggtccagatggacatgtccaaaccagacctgaccgttgccctcagggacatccgtatgcagtacgagggtatcgcagccaagaacatctctgaggcagaggactggtacaagtccaaggtgtcagacctgaaccaggctgtgaacaagaacaacgatgctctgagacaggccaaacaggagagcatggagttcaggcatcagatccagtcctacacctgtgagatcgactctctcaagggaaccaacgagtctctgctgaggcagatgagggagatggaggatcgtctgggaaacgaggctggaggttaccaggactccgtcacccgtctggaggctgagatcgccaagatgaaggatgagatggctcgtcacctcagagagtaccaggacctcctcaatgtcaagatggccctggatatagagatcgctacctacaggaagctactggagggagaggagagccgaatcactgtatctggctccaagtcttctcactctggctcccactctgctgcctcattatattccactgttggattcagagagaccagcccagacgtcggacgatcagcagaggtccactccaagaagactgtcctgatcaagaccatcgagacccgcgatggagaggtcgtcagcgag

SEQ ID NO: 56 - protein, 459 aa, synthetic construct
  REGION 1..459: note = Desmin (fragment)
ISSYRRTFGSGIGSTPGMSSMFSHGGRGSSGSAHMSSRVYEMTKSSARPSYSSGSIRSSSGGAMRSYAGMGEKLDFNLADATNRDFLDTRTNEKAELQHLNDRFASYIEKVRFLEQQNATLVVEIERLRGHEPTRVAEMYEEEMRELRRQVDGMSNDRARMEVERDNLADDLQKLKLRLQEVIHQREEAENNLSAFRADVDSATLARLDLERRIESLQEEITFLKKIHEEEIHELTSQMQETSVQVQMDMSKPDLTVALRDIRMQYEGIAAKNISEAEDWYKSKVSDLNQAVNKNNDALRQAKQESMEFRHQIQSYTCEIDSLKGTNESLLRQMREMEDRLGNEAGGYQDSVTRLEAEIAKMKDEMARHLREYQDLLNVKMALDIEIATYRKLLEGEESRITVSGSKSSHSGSHSAASLYSTVGFRETSPDVGRSAEVHSKKTVLIKTIETRDGEVVSE

SEQ ID NO: 57 - protein, 17 aa, synthetic construct
  REGION 1..17: note = A part of Desmin (fragment)
DEMARHLREYQDLLNVK

SEQ ID NO: 58 - protein, 21 aa, synthetic construct
  REGION 1..21: note = A part of Desmin (fragment)
LDFNLADATNRDFLDTRTNEK

SEQ ID NO: 59 - protein, 10 aa, synthetic construct
  REGION 1..10: note = A part of Desmin (fragment)
NISEAEDWYK

SEQ ID NO: 60 - DNA, 822 bp, synthetic construct
  misc_feature 1..822: note = Capping protein (Actin filament) muscle Z-line beta
  source 1..822: mol_type = other DNA
atgaatgaccagcagctggactgtgctctggacctgatgaggcgtcttcctccccagcagatcgagaagaatctcagtgacctcatcgacctggtgcccagtctgtgtgaggacctactctcctctgtggaccagcctctgaagatcgcccgggacaaggtagtggggaaagactacctgctctgtgactataacagagacggtgactcctacagatccccatggagtaataagtatgagccacccatcgacgatggtgccatgccatctgcccgcctgcgcaaactagaggtggaagccaacaatgcctttgaccagtatagagacctgtactttgagggtggcgtatcgtctgtgtatctgtgggacttggatcatggctttgctggggtcatcctcatcaagaaggctggagacggctccaagaagatcaaaggctgctgggactccatccatgtggtggaggtgcaggagaagtccagcggacggaccgctcactacaaactcacctccaccgtcatgctgtggctccagacgaccaaggccgggtctggaaccatgaacctgggtggcagtctgacaagacagatggagaaagacgagacagttggagagtcttccccacatattgccaacatcggccgcctggtggaggatatggagaataagattcgctccactctcaacgagatctactttgggaagaccaaggacatcgtcaatggtttaagatctattgactctctgcctgataaccaaaagtaccggcagctccagaaggagctgtctcaggtccttacccagcgccagatcttcattgac

SEQ ID NO: 61 - protein, 274 aa, synthetic construct
  REGION 1..274: note = Capping protein (Actin filament) muscle Z-line beta
MNDQQLDCALDLMRRLPPQQIEKNLSDLIDLVPSLCEDLLSSVDQPLKIARDKVVGKDYLLCDYNRDGDSYRSPWSNKYEPPIDDGAMPSARLRKLEVEANNAFDQYRDLYFEGGVSSVYLWDLDHGFAGVILIKKAGDGSKKIKGCWDSIHVVEVQEKSSGRTAHYKLTSTVMLWLQTTKAGSGTMNLGGSLTRQMEKDETVGESSPHIANIGRLVEDMENKIRSTLNEIYFGKTKDIVNGLRSIDSLPDNQKYRQLQKELSQVLTQRQIFID

SEQ ID NO: 62 - protein, 24 aa, synthetic construct
  REGION 1..24: note = Capping protein (Actin filament) muscle Z-line beta fragment
DETVGESSPHIANIGRLVEDMENK

SEQ ID NO: 63 - protein, 17 aa, synthetic construct
  REGION 1..17: note = Capping protein (Actin filament) muscle Z-line beta fragment
DIVNGLRSIDSLPDNQK

SEQ ID NO: 64 - protein, 14 aa, synthetic construct
  REGION 1..14: note = Capping protein (Actin filament) muscle Z-line beta fragment
ELSQVLTQRQIFID

SEQ ID NO: 65 - protein, 12 aa, synthetic construct
  REGION 1..12: note = Capping protein (Actin filament) muscle Z-line beta fragment
IRSTLNEIYFGK

SEQ ID NO: 66 - protein, 13 aa, synthetic construct
  REGION 1..13: note = Capping protein (Actin filament) muscle Z-line beta fragment
LTSTVMLWLQTTK

SEQ ID NO: 67 - protein, 10 aa, synthetic construct
  REGION 1..10: note = Capping protein (Actin filament) muscle Z-line beta fragment
SIDSLPDNQK

SEQ ID NO: 68 - protein, 17 aa, synthetic construct
  REGION 1..17: note = Capping protein (Actin filament) muscle Z-line beta fragment
YEPPIDDGAMPSARLRK

SEQ ID NO: 69 - DNA, 5799 bp, synthetic construct
  misc_feature 1..5799: note = myosin heavy chain, fast skeletal muscle-like
  source 1..5799: mol_type = other DNA
atgagtacggacgcggagatgcaagtctacggcaaggctgccatataccttcgtaagtctgagaaggagaggatggaggcacaagccatgccctttgattcaaagaacgcctgctatgtgacagacaaggtggagctgtaccttaagggtttagtcactgccagggccgacgggaagtgtactgtgacagctttagactttcgtgaaggaaaagagttcaaagatgcagacatctatgagatgaacccccctaagtacgacaagattgaggacatggccatgatgacctacctgaatgaagcctctgtgttgtataacctcaaagagcgttatgcagcatggatgatctatacctactctgggctcttctgtgccacggtgaacccctacaagtggctcccagtgtacgacgaagaggttgtcaacgcctacagagggaagaagagggtggaggctccaccacatatcttctccgtctctgacaacgcctttcagttcatgatgattgataaggagaaccagtccgtcctgattactggagaatccggtgcaggaaagactgtcaacaccaagcgtgtcatccagtactttgccaccattgcagtgtctggtggcaagaaggaagcagaccccaacaaaatgcaggggtctcttgaggatcagatcattgcagctaaccctctgctagagtcttacggtaatgccaagacagtgaggaacgacaactcgtctcgctttggtaaattcatcaggattcacttccaagctggtaaactggctaaagctgacattgagacctacctgctggagaagtccagagtgtccttccaactgcccgatgagagaggctaccacatcttcttccagatgatgacaggccacaaacctgagctagttgaattggcactcctcaccaccaacccctacgacttccccatgtgcagccagggtcagattgctgtggccagcatcaacgacaatgaagagctggatgccacagatgaagccattacaatcctgggcttcactaatgaggagaagcttggaatatacaagctgacaggagctgtagtgcaccatggcaacttgaaattcaagcagaagcagcgtgaggagcaggccgagccagacggcacagaggtggctgataaaatcgcctacctgctgggcctgaactcagctgagatgttgaaagctctgtgctaccccagagtgaaggtcggcaacgagtatgtgaccaagggacagactgtggctcaggttaataactcagtcagtgctctggccaagtccatctatgagaggatgttcttgtggatggtcatccgtatcaatgagatgttggacaccaagaatccaaggcagttctatatcggtgtgcttgacattgccgggtttgagatctttgattacaacagcatggagcagctgtgcatcaacttcaccaatgagaaactgcaacagtttttcaaccacaccatgttcgtcctggagcaagaggagtacaagaaggagggaatcgtctgggccttcattgacttcggcatggatttggctgcctgcattgagcttattgagaagccattgggcatcttctccatccttgaagaggagtgcatgttccccaagtcttcagacactaccttcaaggacaagctgtacgcccagcatcttggtaaaacaaaggcgtttgagaagcccaagcctgccaaaggcaaggcagaggcccacttctccctggtgcattacgccggtactgtggactacaacatcactggctggctggagaagaacaaggaccccttgaacgactcagtttgtcaactgtatgggaagtccggagtcaaaattttggctgccctgtatccccctccccctcctgaggataaagccaagaaaggaggcaagaagaagggtggttccatgcagactgtgtcctcccagttcagggagaacttacataagctgatgaccaacttgaggagcactcatcctcactttgtgcgctgcctgatccccaacgagtcaaagactccaggtctgatggagaacttcctggttatccaccagctcaggtgtaatggtgtactggagggtatcaggatctgcagaaagggcttccccagcagaatcatctatgctgacttcaagcaaaggtacaaagtactgaatgccagcgtcatccctgagggccagttcatggacaacaagaaggcttctgagaagctgcttggatccattgatgtgaatcacgaggattacaagtttggacacaccaaggtgttcttcaaagccggtctgctgggtgtcctggaggagatgagagatgagaagctggcctctctagtcggcatgctccaggctctcagccgtggattcctcatgaggagagagttcaccaagatgatggagaggagagaatcaatttactccatccagtacaacatccgctcattcatgaatgtgaaaacctggccatggatgaagttgtacttcaagatcaagcccctgctgcagagcgctgagactgagaaggagctggccaacatgaaggagaactatgagaaaatgaagacagacctggccaaggctctgtctacaaagaagcaaatggaggagaagttggtgtccctgacgcaggagaagaacgacctggcactccaagtcgcatctgaaggagagagtctgaacgatgctgaggaaaggtgcgaggggctcatcaagagcaagatccagcaggaggccaaactcaaagagacgaccgagaggctggaggatgaggaggagatcaatgctgagttgactgccaagaagaggaagctggaggatgagtgctctgagctgaagaaggacattgatgatctggagctcaccctggccaaagtggagaaggagaagcacgccactgaaaacaaggttaaaaacctgacagaggagatggcgtctatggatgagagtgttgccaagctgaccaaggagaagaaagccctacaagaggcccaccagcagacactggatgacctgcaggcagaggaggacaaagtcaacactctgaccaaggccaagaccaagctggaacagcaagtggacgaccttgagggttctctggagcaagagaagaagctccgcatggaccttgagagatccaagagaaagctggagggagatctgaaactggcccaggagtccataatggacctggagaatgacaagcagcaagctgatgagaaaatcaagaagaaggagtttgagaccactcagctcctcagcaaggttgaggatgagcagtctctgggagctcagctgcagaagaagatcaaggaactccaggcccgtattgaggaactggaggaggaaattgaggctgagcgtgctgccagggctaaggttgagaagcagagggctgatctctccagggaacttgaggagatcagcgagaggctggaggaggccggaggcgccactgctgctcagattgagatgaacaagaagcgtgaggctgagttccagaagctgcgtcgtgatcttgaagagtccaccctgcagcatgaggccacagccgccgctctgcgcaagaagcaggccgacagtgtggctgagctcggggagcagatcgacaacctgcagcgcgtcaagcagaagctggagaaggagaagagcgagtacaagatggagattgatgacctctccagcaacatggaggccgttgccaaggctaagggcaatctagagaagatgtgccgtactcttgaggaccagctgagcgagctcaagactaagaatgatgagaatgttcgccaggtcaacgacatcagcggacagagggccagactcctgacagaaaatggtgagtttggtaggcagctggaggagaaggaagccctggtgtctcagctgaccagaggcaaacaggccttcacccagcaggtggaggagctgaagagggcgattgaggaggaggtcaaggctaaaaatgcactggcccacggagttcagtctgcccgccatgactgtgacctcctgagggagcagtttgaggaggagcaggaggccaaggcagagctgcaacgcggcatgtccaaggccaatagtgaggtggctcagtggaggactaagtatgaaactgatgccatccagcgcacagaggagctggaggaggccaagaagaagctggcccagcgtctgcaggatgccgaggagaccattgaggcgaccaactccaagtgcgcctccctggagaagaccaagcagagactacagggagaggtggaggacctcatgattgatgttgagagagccaacgcattggccgccaacctcgacaagaagcagaggaactttgacaaggttctggcagagtggaagcagaagtatgaggagggtcaggctgagctggaaggagctcagaaggaggctcgctctatgagcactgagctcttcaagatgaagaactcctacgaggaggctctggatcatctggagactctgaagagagagaacaagaacctgcaacaggagatctctgaccttactgagcagattggagagactggcaagagcatccatgagctggagaaggccaagaagaccgtggagacagagaagtctgagatccagaccgctctggaggaggctgagggcacactggagcacgaggaatccaagattctgcgtgtgcagctggagctgaaccagatcaagggtgaggtggacaggaagatcgctgagaaggacgaggagatggagcagatcaagaggaacagccagaggatggttgactccatgcagagcaccctggactctgaggtcaggagcaggaatgatgccctgagggtgaagaagaagatggagggagacctgaacgagatggagatccagctgagccactccaacaggcaggccgctgaggcccagaaacagctgaggaatgtccagggacagctcaaggatgcccaattgcaccttgatgatgccgtccgtgtcgcagaggacatgaaggagcaggcagccatggtggagcgcagaaacggtctgatggtggctgagatcgaggagctgagagttgctctggagcagacagagagaggccgcaaagtggctgagactgagctggtagacgccagcgagcgtgttggactgctgcactcccagaacaccagccttctgaacaccaagaagaagctggagactgacctggtgcaggtgcagggagaggtggatgacatcgtccaggaggccaggaatgcagaagagaaggccaagaaggcaatcactgatgcggcaatgatggctgaggagctgaagaaggagcaggacaccagctctcacctggagagaatgaagaagaacctggagatcacagtcaaggacctgcagcaccgcctggatgaggctgagaatctggccatgaagggaggcaagaaacaactccagaaactggaatccagggtgcgtgagcttgagactgaggtggaggctgagcagagaagaggtgtagacgcggtaaagggagtccgcaagtatgagcgcagagtcaaggagctcacttaccagactgaggaggataagaagaatgttaacagacttcaggacctggtagataagctgcagatgaaagtgaaggcctacaagaggcacgctgaggaagcggaggaagcagcaaaccagcacatgtctaagttcaggaaggttcagcatgagctggaggaggctgaggagcgtgctgacatcgctgagactcaggtcaacaagctcagagccaagacccgtgactctggaaagggaaaagaagttgctgaa

SEQ ID NO: 70 - protein, 1933 aa, synthetic construct
  REGION 1..1933: note = myosin heavy chain, fast skeletal muscle-like
MSTDAEMQVYGKAAIYLRKSEKERMEAQAMPFDSKNACYVTDKVELYLKGLVTARADGKCTVTALDFREGKEFKDADIYEMNPPKYDKIEDMAMMTYLNEASVLYNLKERYAAWMIYTYSGLFCATVNPYKWLPVYDEEVVNAYRGKKRVEAPPHIFSVSDNAFQFMMIDKENQSVLITGESGAGKTVNTKRVIQYFATIAVSGGKKEADPNKMQGSLEDQIIAANPLLESYGNAKTVRNDNSSRFGKFIRIHFQAGKLAKADIETYLLEKSRVSFQLPDERGYHIFFQMMTGHKPELVELALLTTNPYDFPMCSQGQIAVASINDNEELDATDEAITILGFTNEEKLGIYKLTGAVVHHGNLKFKQKQREEQAEPDGTEVADKIAYLLGLNSAEMLKALCYPRVKVGNEYVTKGQTVAQVNNSVSALAKSIYERMFLWMVIRINEMLDTKNPRQFYIGVLDIAGFEIFDYNSMEQLCINFTNEKLQQFFNHTMFVLEQEEYKKEGIVWAFIDFGMDLAACIELIEKPLGIFSILEEECMFPKSSDTTFKDKLYAQHLGKTKAFEKPKPAKGKAEAHFSLVHYAGTVDYNITGWLEKNKDPLNDSVCQLYGKSGVKILAALYPPPPPEDKAKKGGKKKGGSMQTVSSQFRENLHKLMTNLRSTHPHFVRCLIPNESKTPGLMENFLVIHQLRCNGVLEGIRICRKGFPSRIIYADFKQRYKVLNASVIPEGQFMDNKKASEKLLGSIDVNHEDYKFGHTKVFFKAGLLGVLEEMRDEKLASLVGMLQALSRGFLMRREFTKMMERRESIYSIQYNIRSFMNVKTWPWMKLYFKIKPLLQSAETEKELANMKENYEKMKTDLAKALSTKKQMEEKLVSLTQEKNDLALQVASEGESLNDAEERCEGLIKSKIQQEAKLKETTERLEDEEEINAELTAKKRKLEDECSELKKDIDDLELTLAKVEKEKHATENKVKNLTEEMASMDESVAKLTKEKKALQEAHQQTLDDLQAEEDKVNTLTKAKTKLEQQVDDLEGSLEQEKKLRMDLERSKRKLEGDLKLAQESIMDLENDKQQADEKIKKKEFETTQLLSKVEDEQSLGAQLQKKIKELQARIEELEEEIEAERAARAKVEKQRADLSRELEEISERLEEAGGATAAQIEMNKKREAEFQKLRRDLEESTLQHEATAAALRKKQADSVAELGEQIDNLQRVKQKLEKEKSEYKMEIDDLSSNMEAVAKAKGNLEKMCRTLEDQLSELKTKNDENVRQVNDISGQRARLLTENGEFGRQLEEKEALVSQLTRGKQAFTQQVEELKRAIEEEVKAKNALAHGVQSARHDCDLLREQFEEEQEAKAELQRGMSKANSEVAQWRTKYETDAIQRTEELEEAKKKLAQRLQDAEETIEATNSKCASLEKTKQRLQGEVEDLMIDVERANALAANLDKKQRNFDKVLAEWKQKYEEGQAELEGAQKEARSMSTELFKMKNSYEEALDHLETLKRENKNLQQEISDLTEQIGETGKSIHELEKAKKTVETEKSEIQTALEEAEGTLEHEESKILRVQLELNQIKGEVDRKIAEKDEEMEQIKRNSQRMVDSMQSTLDSEVRSRNDALRVKKKMEGDLNEMEIQLSHSNRQAAEAQKQLRNVQGQLKDAQLHLDDAVRVAEDMKEQAAMVERRNGLMVAEIEELRVALEQTERGRKVAETELVDASERVGLLHSQNTSLLNTKKKLETDLVQVQGEVDDIVQEARNAEEKAKKAITDAAMMAEELKKEQDTSSHLERMKKNLEITVKDLQHRLDEAENLAMKGGKKQLQKLESRVRELETEVEAEQRRGVDAVKGVRKYERRVKELTYQTEEDKKNVNRLQDLVDKLQMKVKAYKRHAEEAEEAANQHMSKFRKVQHELEEAEERADIAETQVNKLRAKTRDSGKGKEVAE

SEQ ID NO: 71 - protein, 7 aa, synthetic construct
  REGION 1..7: note = myosin heavy chain, fast skeletal muscle-like fragment
AAIYLRK

SEQ ID NO: 72 - protein, 10 aa, synthetic construct
  REGION 1..10: note = myosin heavy chain, fast skeletal muscle-like fragment
ADIETYLLEK

SEQ ID NO: 73 - protein, 19 aa, synthetic construct
  REGION 1..19: note = myosin heavy chain, fast skeletal muscle-like fragment
ALQEAHQQTLDDLQAEEDK

SEQ ID NO: 74 - protein, 11 aa, synthetic construct
  REGION 1..11: note = myosin heavy chain, fast skeletal muscle-like fragment
DIDDLELTLAK

SEQ ID NO: 75 - protein, 11 aa, synthetic construct
  REGION 1..11: note = myosin heavy chain, fast skeletal muscle-like fragment
EALVSQLTRGK

SEQ ID NO: 76 - protein, 10 aa, synthetic construct
  REGION 1..10: note = myosin heavy chain, fast skeletal muscle-like fragment
EFETTQLLSK

SEQ ID NO: 77 - protein, 10 aa, synthetic construct
  REGION 1..10: note = myosin heavy chain, fast skeletal muscle-like fragment
ELTYQTEEDK

SEQ ID NO: 78 - protein, 15 aa, synthetic construct
  REGION 1..15: note = myosin heavy chain, fast skeletal muscle-like fragment
ENQSVLITGESGAGK

SEQ ID NO: 79 - protein, 19 aa, synthetic construct
  REGION 1..19: note = myosin heavy chain, fast skeletal muscle-like fragment
ETTERLEDEEEINAELTAK

SEQ ID NO: 80 - protein, 10 aa, synthetic construct
  REGION 1..10: note = myosin heavy chain, fast skeletal muscle-like fragment
FIRIHFQAGK

SEQ ID NO: 81 - protein, 12 aa, synthetic construct
  REGION 1..12: note = myosin heavy chain, fast skeletal muscle-like fragment
GFPSRIIYADFK

SEQ ID NO: 82 - protein, 10 aa, synthetic construct
  REGION 1..10: note = myosin heavy chain, fast skeletal muscle-like fragment
GLVTARADGK

SEQ ID NO: 83 - protein, 16 aa, synthetic construct
  REGION 1..16: note = myosin heavy chain, fast skeletal muscle-like fragment
GQTVAQVNNSVSALAK

SEQ ID NO: 84 - protein, 12 aa, synthetic construct
  REGION 1..12: note = myosin heavy chain, fast skeletal muscle-like fragment
IKPLLQSAETEK

SEQ ID NO: 85 - protein, 14 aa, synthetic construct
  REGION 1..14: note = myosin heavy chain, fast skeletal muscle-like fragment
ILAALYPPPPPEDK

SEQ ID NO: 86 - protein, 12 aa, synthetic construct
  REGION 1..12: note = myosin heavy chain, fast skeletal muscle-like fragment
ILRVQLELNQIK

SEQ ID NO: 87 - protein, 18 aa, synthetic construct
  REGION 1..18: note = myosin heavy chain, fast skeletal muscle-like fragment
LAQRLQDAEETIEATNSK

SEQ ID NO: 88 - protein, 16 aa, synthetic construct
  REGION 1..16: note = myosin heavy chain, fast skeletal muscle-like fragment
LEQQVDDLEGSLEQEK

SEQ ID NO: 89 - protein, 24 aa, synthetic construct
  REGION 1..24: note = myosin heavy chain, fast skeletal muscle-like fragment
LESRVRELETEVEAEQRRGVDAVK

SEQ ID NO: 90 - protein, 13 aa, synthetic construct
  REGION 1..13: note = myosin heavy chain, fast skeletal muscle-like fragment
LLGSIDVNHEDYK

SEQ ID NO: 91 - protein, 21 aa, synthetic construct
  REGION 1..21: note = myosin heavy chain, fast skeletal muscle-like fragment
LRRDLEESTLQHEATAAALRK

SEQ ID NO: 92 - protein, 12 aa, synthetic construct
  REGION 1..12: note = myosin heavy chain, fast skeletal muscle-like fragment
LTGAVVHHGNLK

SEQ ID NO: 93 - protein, 32 aa, synthetic construct
  REGION 1..32: note = myosin heavy chain, fast skeletal muscle-like fragment
NDENVRQVNDISGQRARLLTENGEFGRQLEEK

SEQ ID NO: 94 - protein, 7 aa, synthetic construct
  REGION 1..7: note = myosin heavy chain, fast skeletal muscle-like fragment
NLEITVK

SEQ ID NO: 95 - protein, 18 aa, synthetic construct
  REGION 1..18: note = myosin heavy chain, fast skeletal muscle-like fragment
NLQQEISDLTEQIGETGK

SEQ ID NO: 96 - protein, 14 aa, synthetic construct
  REGION 1..14: note = myosin heavy chain, fast skeletal muscle-like fragment
NSYEEALDHLETLK

SEQ ID NO: 97 - protein, 19 aa, synthetic construct
  REGION 1..19: note = myosin heavy chain, fast skeletal muscle-like fragment
QADSVAELGEQIDNLQRVK

SEQ ID NO: 98 - protein, 11 aa, synthetic construct
  REGION 1..11: note = myosin heavy chain, fast skeletal muscle-like fragment
QAFTQQVEELK

SEQ ID NO: 99 - protein, 16 aa, synthetic construct
  REGION 1..16: note = myosin heavy chain, fast skeletal muscle-like fragment
QREEQAEPDGTEVADK

SEQ ID NO: 100 - protein, 8 aa, synthetic construct
  REGION 1..8: note = myosin heavy chain, fast skeletal muscle-like fragment
RAIEEEVK

SEQ ID NO: 101 - protein, 20 aa, synthetic construct
  REGION 1..20: note = myosin heavy chain, fast skeletal muscle-like fragment
SEIQTALEEAEGTLEHEESK

SEQ ID NO: 102 - protein, 12 aa, synthetic construct
  REGION 1..12: note = myosin heavy chain, fast skeletal muscle-like fragment
TVRNDNSSRFGK

SEQ ID NO: 103 - protein, 27 aa, synthetic construct
  REGION 1..27: note = myosin heavy chain, fast skeletal muscle-like fragment
VAETELVDASERVGLLHSQNTSLLNTK

SEQ ID NO: 104 - protein, 13 aa, synthetic construct
  REGION 1..13: note = myosin heavy chain, fast skeletal muscle-like fragment
VEDEQSLGAQLQK

SEQ ID NO: 105 - protein, 8 aa, synthetic construct
  REGION 1..8: note = myosin heavy chain, fast skeletal muscle-like fragment
VGNEYVTK

SEQ ID NO: 106 - protein, 21 aa, synthetic construct
  REGION 1..21: note = myosin heavy chain, fast skeletal muscle-like fragment
VQHELEEAEERADIAETQVNK

SEQ ID NO: 107 - protein, 13 aa, synthetic construct
  REGION 1..13: note = myosin heavy chain, fast skeletal muscle-like fragment
YEEGQAELEGAQK

SEQ ID NO: 108 - protein, 16 aa, synthetic construct
  REGION 1..16: note = myosin heavy chain, fast skeletal muscle-like fragment
YETDAIQRTEELEEAK

SEQ ID NO: 109 - DNA, 2532 bp, synthetic construct
  misc_feature 1..2532: note = glycogen phosphorylase, muscle form-like
  source 1..2532: mol_type = other DNA
atgtcaaaaccattgtcagaccacgataaaaggaagcagatctccgtgagaggtcttgctggtgtggaaaatgtcgcagaactgaaggtcgctttcaacaggcatctccattttacactggtcaaggacagaaatgtggcaaacaaacgggattactactttgctctcgccaacaccgtgcgtgaccacttggtgggcaggtggatcagaacccagcagtactactatgagaaagatcccaaacgtgtgtactacatctccctggagttttatatgggtcgcaccctgcagaacactatggtgaacctggccctggagaacgcttgtgatgaggccatataccagctgggtctggacatggaggagctggaggacatggaagaggatgctggcctgggaaacggtggtcttggccgtcttgccgcatgcttcctggactcaatggcttctctaggccttgctgcctatggctatggtatccgctatgagtttggcatcttcaatcagaagatcgtcaatggctggcaggttgaggaggccgatgattggttgcgttacggcaacccctgggagaaggcccgccctgagtacatgcgccccgtcaagttctatggcaggaccgagcacaccccagatggtgtgaaatgggttgacactcaggtagtgttggctctgccatatgacacccctattcccggctacagaaacaacattgtcaacaccatgagactgtggtctgctaaggccccatgcgacttcaacctgaaagacttcaacgttggtggctacattcaggctgtgttggacagaaacttgtgtgagaacatttcccgcgtgctgtaccccaatgataatttctttgagggcaaggagctgcgtctgaagcaggagtactttgtggtggccgccactctgcaggacgtcgtccgtcgtttcaaggcctctaagtttggctccagagagattgtccgcacagacttcgccgagctaccaaacaaagttgccatccagctgaatgacactcaccctgccatggctattcctgagctgatgagggtcctggttgatgaggagaagctgggttgggacaaggcctgggacgtgtgtgtccgtacctgtgcctacacaaaccacaccgtgctgcctgaggccctggagcgctggcccattgacctgttccatcacctgctgccacgtcacctggagatcatctacgagatcaaccgtcgcttcatggagtatgttgcctcgaagttccctggagacaacgaccgtctgcgtcgcatgtccctgattgaggagggaggatgcaagaaagtcaacatggctcatctgtgtatcgttggttcccatgctgtcaacggcgtggcccgcatccactctgagatcctcatcgccactctgttcaaggacttctatgagttggacccacacaagtttcagaacaagaccaatgggatcaccccccgtcgctggctggttatgtgcaaccccggcttggcggaggtcatcgcagagaaaattggagaggagtttatccgtgaccttgaccagcttaagcgactgttgaagttcgttaatgatgatgctttcatccgtgacatcgccaaagtcaagcaggagaacaagctgaagttcgctgtgcacctggaagagcactacaaggtcaagatcaacccccagtccatgtttgacttccaagtcaaaagaatccacgagtacaaaagacagctgctcaactgtctgcacatgatcacctactacaaccgtatcaagaaggagcccaacaagcactggaccccaagaaccatcatggtcggaggaaaggctgcaccaggctaccacacagccaagatgatcatccgtctcatcacagctatcggtgaggttgtcaaccacgaccccgttgtcggcgaccgcctcaaagttatcttcctggagaactacagagtcaccctggctgagaaagccatcccctctgccgacctgtctgagcagatctctacagctggcaccgaggcctctggcactggtaacatgaagttcatgctgaacggtgctcttaccattggcaccatggatggagccaatgtggagatggccgaggaggccggagaggaaaacttcttcatcttcggtatgagagtggaggatgtggacgcaatggacgccggcaaaggataccacgcctctgagtactacaaccgtattcccgagctgaagcaggccatggaccagattgctggcggcttcttcagtcccaagcagcctgacctcttcaaggaacttgtggacctgctgatgcaccatgacaggttcaaggtgtttgctgactacgaagcctacatcaaatgccaggacaaggtcaaccaactgtacaagaatcccaaggaatggaccaagatggtgatccataacattgcgggctgtggtaaattctccagcgaccgcaccattgcccagtacgcccgagagatctggggcatggagcccagcctggagaagatccctgcccccgatgagaaactcaaa

SEQ ID NO: 110 - protein, 844 aa, synthetic construct
  REGION 1..844: note = glycogen phosphorylase, muscle form-like
MSKPLSDHDKRKQISVRGLAGVENVAELKVAFNRHLHFTLVKDRNVANKRDYYFALANTVRDHLVGRWIRTQQYYYEKDPKRVYYISLEFYMGRTLQNTMVNLALENACDEAIYQLGLDMEELEDMEEDAGLGNGGLGRLAACFLDSMASLGLAAYGYGIRYEFGIFNQKIVNGWQVEEADDWLRYGNPWEKARPEYMRPVKFYGRTEHTPDGVKWVDTQVVLALPYDTPIPGYRNNIVNTMRLWSAKAPCDFNLKDFNVGGYIQAVLDRNLCENISRVLYPNDNFFEGKELRLKQEYFVVAATLQDVVRRFKASKFGSREIVRTDFAELPNKVAIQLNDTHPAMAIPELMRVLVDEEKLGWDKAWDVCVRTCAYTNHTVLPEALERWPIDLFHHLLPRHLEIIYEINRRFMEYVASKFPGDNDRLRRMSLIEEGGCKKVNMAHLCIVGSHAVNGVARIHSEILIATLFKDFYELDPHKFQNKTNGITPRRWLVMCNPGLAEVIAEKIGEEFIRDLDQLKRLLKFVNDDAFIRDIAKVKQENKLKFAVHLEEHYKVKINPQSMFDFQVKRIHEYKRQLLNCLHMITYYNRIKKEPNKHWTPRTIMVGGKAAPGYHTAKMIIRLITAIGEVVNHDPVVGDRLKVIFLENYRVTLAEKAIPSADLSEQISTAGTEASGTGNMKFMLNGALTIGTMDGANVEMAEEAGEENFFIFGMRVEDVDAMDAGKGYHASEYYNRIPELKQAMDQIAGGFFSPKQPDLFKELVDLLMHHDRFKVFADYEAYIKCQDKVNQLYKNPKEWTKMVIHNIAGCGKFSSDRTIAQYAREIWGMEPSLEKIPAPDEKLK

SEQ ID NO: 111 - protein, 9 aa, synthetic construct
  REGION 1..9: note = glycogen phosphorylase, muscle form-like fragment
DFYELDPHK

SEQ ID NO: 112 - protein, 10 aa, synthetic construct
  REGION 1..10: note = glycogen phosphorylase, muscle form-like fragment
FAVHLEEHYK

SEQ ID NO: 113 - protein, 13 aa, synthetic construct
  REGION 1..13: note = glycogen phosphorylase, muscle form-like fragment
FYGRTEHTPDGVK

SEQ ID NO: 114 - protein, 15 aa, synthetic construct
  REGION 1..15: note = glycogen phosphorylase, muscle form-like fragment
GYHASEYYNRIPELK

SEQ ID NO: 115 - protein, 13 aa, synthetic construct
  REGION 1..13: note = glycogen phosphorylase, muscle form-like fragment
IGEEFIRDLDQLK

SEQ ID NO: 116 - protein, 14 aa, synthetic construct
  REGION 1..14: note = glycogen phosphorylase, muscle form-like fragment
QAMDQIAGGFFSPK

SEQ ID NO: 117 - protein, 17 aa, synthetic construct
  REGION 1..17: note = glycogen phosphorylase, muscle form-like fragment
QISVRGLAGVENVAELK

SEQ ID NO: 118 - protein, 10 aa, synthetic construct
  REGION 1..10: note = glycogen phosphorylase, muscle form-like fragment
VFADYEAYIK

SEQ ID NO: 119 - protein, 14 aa, synthetic construct
  REGION 1..14: note = glycogen phosphorylase, muscle form-like fragment
VIFLENYRVTLAEK

SEQ ID NO: 120 - DNA, 3399 bp, synthetic construct
  misc_feature 1..3399: note = myosin-binding protein C, fast-type-like
  source 1..3399: mol_type = other DNA
atgcctgaagccgctgatgcagtgaagccggagggcgaggagggagaagcaccagcagaagccgaagaaaatactgcagaagatgatgagccactgcctgaaggagaggagggtgcccatcagtcacaggagattacaggactgtttcttcagcaccctgagactacggttgccataacaggtacggacattgtgtttactgccaaagtggactctactacactatcaaggaaacccaccattaaatggctgaaggggaagtggctggacctgggcagcaaagcaggaaaacacatgcagttcaaagagacttttgacagagccacaaagatctacacctgggacatgaagataatcaaggtggtccctggtgacgctggggcctacaggtgtgaggtcacctccaaagacaagtgtgacagcagcgctttcgacatctccgtggaggctgtgcaaattgaggaggaacatgattctttggccgcattcaaaagaacggatgctggagaggatgaaggcagtttggatttcagtgctttgctgaaagctaccaagaagaagaagaagccagtcgtagatgaggagaaggcagacgtgtgggaaatcctgaagaacgctcaacccagcgagtatgagaagattgcttttgagtacggcattacagacctgaggggtctgctcaagcgactgaagaagatgaagactgttgagcccaagcacagcgacgctttcctaaagagaatggagtctgcctactctgtggataagggcaagaaaatcgtactgcaggtggaagtggttgacccaaatgcccaggtcaaatggttgaagaacggtcaggagataaaatcatcagccaagtacatcatagaatcagttggcaacatcaggacgctcaccatcaacaagtgtagcctggctgacgacgctgcgtacgagtgtgtgattggggaagagaagtccttcacagaggtgtttgtcaaagagcccccggtcaccatcaccaagctgctggatgattaccacgtggtggtgggagagagagtggagtttgaggtggaggtgtctgtggagggagcacacgttaactggatgtttgaggaccaagaactctccagggacacccataagtaccgctttaagaaggacgggttgaagcacatgctcatcatccaagaggctacgctggatgacattggaatgtactggtgcttcaccaacggcgggcgaaccaaaggagagctagaagttgaagagaaagaactggaggtgttgcagaacatcgctgacctgacggtgaaggccgaggaccaggccatgttcaagtgtgaggtgtctgatgagaaggtaacagggaaatggctcaaagatggcgtggaggtgctgcccagcagtcgcatcaagctgacccacatcggaagaatccatcggcttaccatagacgacgtcaagcccgaggacgctggagactatacctttatcccagacggatatgccctctcactgtccgccaaactcaacttcctggaaattaagatcgactatgtcccccgccaagagccacccaagatccatctagacaccaccggcaacatggtttcccagaacaccatcattgtggtggctggcaacaagctgcgcctggacgtggagatctctggagagcctcctcccactgtcgtctgggcaaagggagacacggcaatcacagctgtggaggggcgtgtgaggactgagagccggaaggacctgagctgcttcgtcatagagggggcagagagagaggatgagggcaactacaccatcattgtcaccaaccctgccggagaggacaaggctcatctgtttgtcaagattgttgatgtgcctgactgccctgagaatgtaaagtgtacctcggtgggagaagactgtgccaccatggtctgggacccacccaagttcgacggaggcgcaccagtcacaggctatctcatggagaggaagaagaagggctccaccaggtggacgaagctcaactttgacgtgtacgagggggtgacgtacgaagccaagaggatgattgagggcgtgctctacgagatgagagtgtacgccgtcaatggcattggcatgtcccagcccagcctcaattccaaacccttcatgcccatcgctgcaaccagtgagcctctgggcctcaaggtgcatgatgtcacagacaccacatgtaccctgaagtggctggcccctgagaagatcggtgccggaggcctggacggatacgttattgagtactgcaaggagggagacacggagtgggtggtggcgaacactgagctggtggagagacagaattacacagtccgcaacctccccaccgcggagaagatcaacttcagagtggtggccgtgaacattgctggacgcagcccccctaccggcctgagccagcccgtcactatccgcgagatcgtggaactgcccaagatccgcctccctcgctacttgagacagaagtacattagaagagtgggcgacaaaatcaacctgaccatccccttccagggtaagccacgtcccaaagtgcaatggtacaaggatggggaagagcttgacactcgagttgccagcatacgcaattccgaagtggactctatcctgttcatccgctcggctgagagatcccactctgggaagtacgagttggtcctgcagattgagaacatggaggccagggctattctggaaatcaggatcgtagaggcacctgggcctcccgaggtagtgaaggtcacagacgtttggggcttcaacgctgcgctggagtggaagccaccgaaggacgacggcaactgcgagatcaccggttacacaatacagaaggcggacaagaagacaaatgaatggttcactatctacgagcacaacaggaggacaaactgcacagcctcagacctgatcatgggaaatgagtacatgttccgtgtcttcagcgagaacctcgttggaaagagtgaggatttctgcctcagcaaggacacagccatcatacctaagataggattggagtacaacccacctccattcaaggagaaggatatgcaaagcgcccccaagttcatacagcccctgcttgacaggtctgtggtggcaggctacagtaccaccatcagctctgctgtcaagacttgtcctaaacctaagatcaggtggttgaggaataagattcccctggacgagaaccctaggttcctgatgcagaacaaccagggggttttgaccctgaacatccgcaagcccagtcagtatgacgggggcaagttcacctgcaaggctatcaactctttgggggaggacgtcgtggagtgcaccctactcgtacgagctctcaaggacaaggaagatggagacgagaaa

SEQ ID NO: 121 - protein, 1133 aa, synthetic construct
  REGION 1..1133: note = myosin-binding protein C, fast-type-like
MPEAADAVKPEGEEGEAPAEAEENTAEDDEPLPEGEEGAHQSQEITGLFLQHPETTVAITGTDIVFTAKVDSTTLSRKPTIKWLKGKWLDLGSKAGKHMQFKETFDRATKIYTWDMKIIKVVPGDAGAYRCEVTSKDKCDSSAFDISVEAVQIEEEHDSLAAFKRTDAGEDEGSLDFSALLKATKKKKKPVVDEEKADVWEILKNAQPSEYEKIAFEYGITDLRGLLKRLKKMKTVEPKHSDAFLKRMESAYSVDKGKKIVLQVEVVDPNAQVKWLKNGQEIKSSAKYIIESVGNIRTLTINKCSLADDAAYECVIGEEKSFTEVFVKEPPVTITKLLDDYHVVVGERVEFEVEVSVEGAHVNWMFEDQELSRDTHKYRFKKDGLKHMLIIQEATLDDIGMYWCFTNGGRTKGELEVEEKELEVLQNIADLTVKAEDQAMFKCEVSDEKVTGKWLKDGVEVLPSSRIKLTHIGRIHRLTIDDVKPEDAGDYTFIPDGYALSLSAKLNFLEIKIDYVPRQEPPKIHLDTTGNMVSQNTIIVVAGNKLRLDVEISGEPPPTVVWAKGDTAITAVEGRVRTESRKDLSCFVIEGAEREDEGNYTIIVTNPAGEDKAHLFVKIVDVPDCPENVKCTSVGEDCATMVWDPPKFDGGAPVTGYLMERKKKGSTRWTKLNFDVYEGVTYEAKRMIEGVLYEMRVYAVNGIGMSQPSLNSKPFMPIAATSEPLGLKVHDVTDTTCTLKWLAPEKIGAGGLDGYVIEYCKEGDTEWVVANTELVERQNYTVRNLPTAEKINFRVVAVNIAGRSPPTGLSQPVTIREIVELPKIRLPRYLRQKYIRRVGDKINLTIPFQGKPRPKVQWYKDGEELDTRVASIRNSEVDSILFIRSAERSHSGKYELVLQIENMEARAILEIRIVEAPGPPEVVKVTDVWGFNAALEWKPPKDDGNCEITGYTIQKADKKTNEWFTIYEHNRRTNCTASDLIMGNEYMFRVFSENLVGKSEDFCLSKDTAIIPKIGLEYNPPPFKEKDMQSAPKFIQPLLDRSVVAGYSTTISSAVKTCPKPKIRWLRNKIPLDENPRFLMQNNQGVLTLNIRKPSQYDGGKFTCKAINSLGEDVVECTLLVRALKDKEDGDEK

SEQ ID NO: 122 - protein, 8 aa, synthetic construct
  REGION 1..8: note = myosin-binding protein C, fast-type-like fragment
ADVWEILK

SEQ ID NO: 123 - protein, 14 aa, synthetic construct
  REGION 1..14: note = myosin-binding protein C, fast-type-like fragment
ELEVLQNIADLTVK

SEQ ID NO: 124 - protein, 8 aa, synthetic construct
  REGION 1..8: note = myosin-binding protein C, fast-type-like fragment
EPPVTITK

SEQ ID NO: 125 - protein, 8 aa, synthetic construct
  REGION 1..8: note = myosin-binding protein C, fast-type-like fragment
GELEVEEK

SEQ ID NO: 126 - protein, 7 aa, synthetic construct
  REGION 1..7: note = myosin-binding protein C, fast-type-like fragment
HSDAFLK

SEQ ID NO: 127 - protein, 15 aa, synthetic construct
  REGION 1..15: note = myosin-binding protein C, fast-type-like fragment
IAFEYGITDLRGLLK

SEQ ID NO: 128 - protein, 11 aa, synthetic construct
  REGION 1..11: note = myosin-binding protein C, fast-type-like fragment
IGLEYNPPPFK

SEQ ID NO: 129 - protein, 14 aa, synthetic construct
  REGION 1..14: note = myosin-binding protein C, fast-type-like fragment
INLTIPFQGKPRPK

SEQ ID NO: 130 - protein, 15 aa, synthetic construct
  REGION 1..15: note = myosin-binding protein C, fast-type-like fragment
IVLQVEVVDPNAQVK

SEQ ID NO: 131 - protein, 14 aa, synthetic construct
  REGION 1..14: note = myosin-binding protein C, fast-type-like fragment
LNFDVYEGVTYEAK

SEQ ID NO: 132 - protein, 7 aa, synthetic construct
  REGION 1..7: note = myosin-binding protein C, fast-type-like fragment
LNFLEIK

SEQ ID NO: 133 - protein, 9 aa, synthetic construct
  REGION 1..9: note = myosin-binding protein C, fast-type-like fragment
NAQPSEYEK

SEQ ID NO: 134 - protein, 18 aa, synthetic construct
  REGION 1..18: note = myosin-binding protein C, fast-type-like fragment
RTDAGEDEGSLDFSALLK

SEQ ID NO: 135 - protein, 8 aa, synthetic construct
  REGION 1..8: note = myosin-binding protein C, fast-type-like fragment
SFTEVFVK

SEQ ID NO: 136 - protein, 13 aa, synthetic construct
  REGION 1..13: note = myosin-binding protein C, fast-type-like fragment
VDSTTLSRKPTIK

SEQ ID NO: 137 - DNA, 1551 bp, synthetic construct
  misc_feature 1..1551: note = ATP synthase subunit beta, mitochondrial
  source 1..1551: mol_type = other DNA
atgttaggagctgtgggacgctgctgcaccggggctctccaggcacttaagcctggggtccagcccctgaaggcccttgtaggatcgccatctgtccttggacgcagagactactctgcacctgctgctgctgccgccttcgcccatggcaggatcgtagcggtcatcggtgccgtcgtcgacgtccagttcgatgagggcctcccacccatcctcaatgccctggaagttgctgggcgtgagtccaggctagttctggaggtggcacagcatcttggtgagaacactgtgcgtaccattgccatggatggtactgaaggtcttgtccgtggacagaaggttgtggacactggcgatcccatcagaatcccagtgggtcccgagaccctaggcagaatcatgaatgtcattggagagcccattgatgagagggggccaatctccaccaagcagactgccgccatccacgctgaggccccagagttcactgacatgagtgtggagcaggagatcctggtaactggcatcaaggtggtagatctgctggctccctatgccaagggaggcaaaatcggtctgttcggtggtgctggtgtgggcaagactgtgttgatcatggagctgatcaacaatgtggccaaggcccatggtggttactctgtgtttgccggagtgggagagcgtacccgcgagggaaatgacttgtaccacgagatgattgagtcgggtgtcatcaacctgaaggatgacacctccaaggtggcgctggtgtacggacaaatgaacgagcccccaggcgcccgtgcccgtgtggctctgactggtctgaccgtggcagagtatttccgtgaccaggagggtcaggatgtgctgctcttcatcgataacatcttccgtttcacccaggctggctccgaggtgtctgccctgctgggtcgtatcccctccgctgtgggttaccagcccaccctggccactgacatgggtaccatgcaggagagaatcaccaccaccaagaagggttccatcacctctgtgcaggccatctatgtgccggctgacgatttgactgatcccgcccctgccaccaccttcgctcacttggacgccaccactgtgctgtcccgtgcaatcgctgagctgggtatctaccctgctgtggatcccctggattccacctcccgtatcatggaccccaacattgtcggcgctgagcactatgatgttgctcgtggcgtgcagaagatcctccaggactacaagtccctgcaggatatcattgccatcctgggtatggatgagttgtctgaggaggacaagctgattgtctctcgcgcccgcaagatccagcgtttcctgtcccagcccttccaggtggcggaggtattcaccggtcatgctggcaagctggtgccactcaaggagaccatcagtggcttccagagcattctaaatggtgagtacgatgccctgcccgagcaggctttctacatggttggacccatcgaggaggtggttgagaaggctgcacagatggccaaggatctcgca

SEQ ID NO: 138 - protein, 517 aa, synthetic construct
  REGION 1..517: note = ATP synthase subunit beta, mitochondrial
MLGAVGRCCTGALQALKPGVQPLKALVGSPSVLGRRDYSAPAAAAAFAHGRIVAVIGAVVDVQFDEGLPPILNALEVAGRESRLVLEVAQHLGENTVRTIAMDGTEGLVRGQKVVDTGDPIRIPVGPETLGRIMNVIGEPIDERGPISTKQTAAIHAEAPEFTDMSVEQEILVTGIKVVDLLAPYAKGGKIGLFGGAGVGKTVLIMELINNVAKAHGGYSVFAGVGERTREGNDLYHEMIESGVINLKDDTSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGAEHYDVARGVQKILQDYKSLQDIIAILGMDELSEEDKLIVSRARKIQRFLSQPFQVAEVFTGHAGKLVPLKETISGFQSILNGEYDALPEQAFYMVGPIEEVVEKAAQMAKDLA

SEQ ID NO: 139 - protein, 11 aa, synthetic construct
  REGION 1..11: note = ATP synthase subunit beta, mitochondrial fragment
IGLFGGAGVGK

SEQ ID NO: 140 - protein, 21 aa, synthetic construct
  REGION 1..21: note = ATP synthase subunit beta, mitochondrial fragment
IQRFLSQPFQVAEVFTGHAGK

SEQ ID NO: 141 - protein, 13 aa, synthetic construct
  REGION 1..13: note = ATP synthase subunit beta, mitochondrial fragment
TVLIMELINNVAK

SEQ ID NO: 142 - protein, 10 aa, synthetic construct
  REGION 1..10: note = ATP synthase subunit beta, mitochondrial fragment
VVDLLAPYAK

SEQ ID NO: 143 - DNA, 996 bp, synthetic construct
  misc_feature 1..996: note = L-lactate dehydrogenase A chain-like
  source 1..996: mol_type = other DNA
atgactaccaaggagaagctgatcacccatgtgttggctggtgagcctgttggctcccggagcaaggtgacagttgttggcgtcggcatggttggcatggcctccgcagtcagcgtcctgctcaaggacctgtgcgatgagctgtgcctgattgacgtgatggaagataaactgaagggtgaggtcatggacctgcagcatggcagcctcttctgcaagactcacaagattgtgggcgacaaggactacagtacgactgcccactccaaggtggtggtggtcacagccggtgctcgtcagcaagagggtgagagccgtctgaacctggtgcagcgtaacgtcaacatcttcaaattcataattccccagatcgtcaagtacagccccaacgccatcctgctggtcgtctccaatcctgttgacatcctaacctacgtggcttggaagctgagtggtttcccccgtcaccgcgtcatcggttccggcaccaacctggactctggtcgtttccgccacctgatgggcgagaagctacaccttcacccatccagctgtcacggctggatcattggagaacacggagactccagcgtgcccgtatggagcggtgtgaatgttgccggtgtttccctgaagggcctgaacccagacatgggcacagacgcagacaaggaggactggaagcacgtccacaagatggtggtcgacggtgcctatgaggtcatcaagctgaagggttacacctcctgggctatcggcatgtccgtcgctgacctggttgagagcatcctgaagaacctccacaaagtccaccctgtgtccaccctggtcaagggaatgcacggtgtgaaggaggaggtgtttctcagcgtgccctgcgtgctgggaaacagcggtctgaccgacgtcatccacatgactctgaagcccgaggaggagaagcagctgagcaacagtgccgagaccctatggggcgtacagaaagagctcaccttg

SEQ ID NO: 144 - protein, 332 aa, synthetic construct
  REGION 1..332: note = L-lactate dehydrogenase A chain-like
MTTKEKLITHVLAGEPVGSRSKVTVVGVGMVGMASAVSVLLKDLCDELCLIDVMEDKLKGEVMDLQHGSLFCKTHKIVGDKDYSTTAHSKVVVVTAGARQQEGESRLNLVQRNVNIFKFIIPQIVKYSPNAILLVVSNPVDILTYVAWKLSGFPRHRVIGSGTNLDSGRFRHLMGEKLHLHPSSCHGWIIGEHGDSSVPVWSGVNVAGVSLKGLNPDMGTDADKEDWKHVHKMVVDGAYEVIKLKGYTSWAIGMSVADLVESILKNLHKVHPVSTLVKGMHGVKEEVFLSVPCVLGNSGLTDVIHMTLKPEEEKQLSNSAETLWGVQKELTL

SEQ ID NO: 145 - protein, 8 aa, synthetic construct
  REGION 1..8: note = L-lactate dehydrogenase A chain-like fragment
FIIPQIVK

SEQ ID NO: 146 - protein, 16 aa, synthetic construct
  REGION 1..16: note = L-lactate dehydrogenase A chain-like fragment
LITHVLAGEPVGSRSK

SEQ ID NO: 147 - protein, 14 aa, synthetic construct
  REGION 1..14: note = L-lactate dehydrogenase A chain-like fragment
THVLAGEPVGSRSK

SEQ ID NO: 148 - protein, 9 aa, synthetic construct
  REGION 1..9: note = L-lactate dehydrogenase A chain-like fragment
VHPVSTLVK

SEQ ID NO: 149 - protein, 28 aa, synthetic construct
  REGION 1..28: note = L-lactate dehydrogenase A chain-like fragment
VVVVTAGARQQEGESRLNLVQRNVNIFK

SEQ ID NO: 150 - protein, 15 aa, synthetic construct
  REGION 1..15: note = epitope
SGERLPKPDKGKMRF

SEQ ID NO: 151 - protein, 15 aa, synthetic construct
  REGION 1..15: note = epitope
YSKLRKDDPMGNLNT

SEQ ID NO: 152 - protein, 15 aa, synthetic construct
  REGION 1..15: note = epitope
SCFYHAFAGAEQAET

SEQ ID NO: 153 - protein, 15 aa, synthetic construct
  REGION 1..15: note = epitope
TKLRLSNRPAFMPSE

SEQ ID NO: 154 - protein, 15 aa, synthetic construct
  REGION 1..15: note = epitope
SDKTYITVEELRREL

SEQ ID NO: 155 - protein, 15 aa, synthetic construct
  REGION 1..15: note = epitope
LDNRLYYMVAEHDSS

SEQ ID NO: 156 - protein, 15 aa, synthetic construct
  REGION 1..15: note = epitope
GGYIQAVLDRNLCEN

SEQ ID NO: 157 - protein, 15 aa, synthetic construct
  REGION 1..15: note = epitope
DRFKVFADYEAYIKS

SEQ ID NO: 158 - protein, 15 aa, synthetic construct
  REGION 1..15: note = epitope
AIMDKKSNIRNMSVI

SEQ ID NO: 159 - protein, 15 aa, synthetic construct
  REGION 1..15: note = epitope
GKSTLTDSLVSKAGI

SEQ ID NO: 160 - protein, 15 aa, synthetic construct
  REGION 1..15: note = epitope
SMVLVKMKEIAEAYL

SEQ ID NO: 161 - protein, 15 aa, synthetic construct
  REGION 1..15: note = heat shock cognate 70 kDa protein epitope
EAEQYKAADDVQRDK

SEQ ID NO: 162 - protein, 15 aa, synthetic construct
  REGION 1..15: note = epitope
KLSDEDKQKILDKCN

SEQ ID NO: 163 - protein, 15 aa, synthetic construct
  REGION 1..15: note = epitope
DTCYYAVAVAKKGTE

SEQ ID NO: 164 - protein, 15 aa, synthetic construct
  REGION 1..15: note = epitope
YSGAFQCLKDGAGEV

SEQ ID NO: 165 - protein, 15 aa, synthetic construct
  REGION 1..15: note = epitope
IYSKLMAVTNFNLFS

SEQ ID NO: 166 - protein, 15 aa, synthetic construct
  REGION 1..15: note = epitope
MSTIRSLTKAQATGV

SEQ ID NO: 167 - protein, 15 aa, synthetic construct
  REGION 1..15: note = epitope
YYGYNGAFRCLVEDA

SEQ ID NO: 168 - protein, 15 aa, synthetic construct
  REGION 1..15: note = epitope
RYVKKVGEKINIVIP

SEQ ID NO: 169 - protein, 15 aa, synthetic construct
  REGION 1..15: note = epitope
SYEMCVKVDDFEDKA

SEQ ID NO: 170 - protein, 15 aa, synthetic construct
  REGION 1..15: note = epitope
KEVIDYKPTPFVEHD

SEQ ID NO: 171 - protein, 15 aa, synthetic construct
  REGION 1..15: note = epitope
NNMDNPKYRMISTGG

SEQ ID NO: 172 - protein, 15 aa, synthetic construct
  REGION 1..15: note = epitope
NDRFASYIEKVRFLE

SEQ ID NO: 173 - protein, 15 aa, synthetic construct
  REGION 1..15: note = epitope
YKSKVSDLNQAVNKN

SEQ ID NO: 174 - protein, 15 aa, synthetic construct
  REGION 1..15: note = epitope
DVGRSAEVHSKKTVL

SEQ ID NO: 175 - protein, 15 aa, synthetic construct
  REGION 1..15: note = epitope
VILIKKAGDGSKKIK

SEQ ID NO: 176 - protein, 15 aa, synthetic construct
  REGION 1..15: note = epitope
IHVVEVQEKSSGRTA

SEQ ID NO: 177 - protein, 15 aa, synthetic construct
  REGION 1..15: note = epitope
IYFGKTKDIVNGLRS

SEQ ID NO: 178 - protein, 15 aa, synthetic construct
  REGION 1..15: note = epitope
DNQKYRQLQKELSQV

SEQ ID NO: 179 - protein, 15 aa, synthetic construct
  REGION 1..15: note = epitope
TVTALDFREGKEFKD

SEQ ID NO: 180 - protein, 15 aa, synthetic construct
  REGION 1..15: note = epitope
YNSMEQLCINFTNEK

SEQ ID NO: 181 - protein, 15 aa, synthetic construct
  REGION 1..15: note = epitope
LYPPPPPEDKAKKGG

SEQ ID NO: 182 - protein, 15 aa, synthetic construct
  REGION 1..15: note = myosin heavy chain, fast skeletal muscle-like epitope
ASMDESVAKLTKEKK

SEQ ID NO: 183 - protein, 15 aa, synthetic construct
  REGION 1..15: note = epitope
EEDKVNTLTKAKTKL

SEQ ID NO: 184 - protein, 15 aa, synthetic construct
  REGION 1..15: note = epitope
KLRMDLERSKRKLEG

SEQ ID NO: 185 - protein, 15 aa, synthetic construct
  REGION 1..15: note = epitope
DIVQEARNAEEKAKK

SEQ ID NO: 186 - protein, 15 aa, synthetic construct
  REGION 1..15: note = epitope
GGYIQAVLDRNLCEN

SEQ ID NO: 187 - protein, 15 aa, synthetic construct
  REGION 1..15: note = epitope
LKATKKKKKPVVDEE

SEQ ID NO: 188 - protein, 15 aa, synthetic construct
  REGION 1..15: note = epitope
YEKIAFEYGITDLRG

SEQ ID NO: 189 - protein, 15 aa, synthetic construct
  REGION 1..15: note = epitope
TDLRGLLKRLKKMKT

SEQ ID NO: 190 - protein, 15 aa, synthetic construct
  REGION 1..15: note = epitope
KKMKTVEPKHSDAFL

SEQ ID NO: 191 - protein, 15 aa, synthetic construct
  REGION 1..15: note = epitope
AYSVDKGKKIVLQVE

SEQ ID NO: 192 - protein, 15 aa, synthetic construct
  REGION 1..15: note = epitope
LSRDTHKYRFKKDGL

SEQ ID NO: 193 - protein, 15 aa, synthetic construct
  REGION 1..15: note = epitope
YTFIPDGYALSLSAK

SEQ ID NO: 194 - protein, 15 aa, synthetic construct
  REGION 1..15: note = epitope
GAPVTGYLMERKKKG

SEQ ID NO: 195 - protein, 15 aa, synthetic construct
  REGION 1..15: note = epitope
RQKYIRRVGDKINLT

SEQ ID NO: 196 - protein, 15 aa, synthetic construct
  REGION 1..15: note = epitope
YTIQKADKKTNEWFT

SEQ ID NO: 197 - protein, 15 aa, synthetic construct
  REGION 1..15: note = epitope
NVAKAHGGYSVFAGV

SEQ ID NO: 198 - protein, 15 aa, synthetic construct
  REGION 1..15: note = epitope
VLAGEPVGSRSKVTV

SEQ ID NO: 199 - protein, 15 aa, synthetic construct
  REGION 1..15: note = epitope
SYGRALQASALKAWG

SEQ ID NO: 200 - protein, 15 aa, synthetic construct
  REGION 1..15: note = epitope
DGDKSRYLGKGTVKA

SEQ ID NO: 201 - protein, 15 aa, synthetic construct
  REGION 1..15: note = epitope
YRHIADLAGHKDVIL

SEQ ID NO: 202 - protein, 15 aa, synthetic construct
  REGION 1..15: note = epitope
NLKNVIKAKYGKDAT

SEQ ID NO: 203 - protein, 15 aa, synthetic construct
  REGION 1..15: note = epitope
YMVYMFKYDSTHGRF

SEQ ID NO: 204 - protein, 15 aa, synthetic construct
  REGION 1..15: note = epitope
PASYDAIKKVVKAAA

SEQ ID NO: 205 - skipped

SEQ ID NO: 206 - protein, 10 aa, synthetic construct
  REGION 1..10: note = epitope
  VARIANT 2..6: note = misc_feature - X can be any amino acid
SXXXXXKPDK

SEQ ID NO: 207 - protein, 10 aa, synthetic construct
  REGION 1..10: note = epitope
SGERLPKPDK

SEQ ID NO: 208-209 - skipped (2 entries)

SEQ ID NO: 210 - protein, 6 aa, synthetic construct
  REGION 1..6: note = epitope
DKGKMR

SEQ ID NO: 211 - skipped

SEQ ID NO: 212 - protein, 10 aa, synthetic construct
  REGION 1..10: note = epitope
  VARIANT 2..5: note = misc_feature - X can be any amino acid
YXXXXKDDPM

SEQ ID NO: 213 - protein, 10 aa, synthetic construct
  REGION 1..10: note = epitope
YSKLRKDDPM

SEQ ID NO: 214 - skipped

SEQ ID NO: 215 - protein, 10 aa, synthetic construct
  REGION 1..10: note = epitope
  VARIANT 3..6: note = misc_feature - X can be any amino acid
  VARIANT 8: note = misc_feature - X can be any amino acid
YSXXXXDXPM

SEQ ID NO: 216 - skipped

SEQ ID NO: 217 - protein, 7 aa, synthetic construct
  REGION 1..7: note = epitope
  VARIANT 2: note = misc_feature - X can be any amino acid
PXGNLNT

SEQ ID NO: 218 - protein, 7 aa, synthetic construct
  REGION 1..7: note = epitope
PMGNLNT

SEQ ID NO: 219 - protein, 15 aa, synthetic construct
  REGION 1..15: note = epitope
  VARIANT 2: note = misc_feature - X can be any amino acid
SXFYHAFAGAEQAET

SEQ ID NO: 220 - protein, 15 aa, synthetic construct
  REGION 1..15: note = epitope
  VARIANT 2: note = misc_feature - X can be any amino acid
  VARIANT 4..6: note = misc_feature - X can be any amino acid
  VARIANT 8: note = misc_feature - X can be any amino acid
  VARIANT 10..13: note = misc_feature - X can be any amino acid
TXLXXXNXPXXXXSE

SEQ ID NO: 221 - protein, 15 aa, synthetic construct
  REGION 1..15: note = epitope
  VARIANT 2: note = misc_feature - X can be any amino acid
  VARIANT 6: note = misc_feature - X can be any amino acid
  VARIANT 10..13: note = misc_feature - X can be any amino acid
TXLRLXNRPXXXXSE

SEQ ID NO: 222 - protein, 12 aa, synthetic construct
  REGION 1..12: note = epitope
  VARIANT 2: note = misc_feature - X can be any amino acid
  VARIANT 5..9: note = misc_feature - X can be any amino acid
  VARIANT 11: note = misc_feature - X can be any amino acid
SXKTXXXXXEXR

SEQ ID NO: 223 - protein, 12 aa, synthetic construct
  REGION 1..12: note = epitope
  VARIANT 5..9: note = misc_feature - X can be any amino acid
SDKTXXXXXELR

SEQ ID NO: 224 - protein, 12 aa, synthetic construct
  REGION 1..12: note = epitope
SDKTYITVEELR

SEQ ID NO: 225-226 - skipped (2 entries)

SEQ ID NO: 227 - protein, 4 aa, synthetic construct
  REGION 1..4: note = epitope
LRRE

SEQ ID NO: 228 - protein, 11 aa, synthetic construct
  REGION 1..11: note = epitope
  VARIANT 1..2: note = misc_feature - X can be any amino acid
  VARIANT 5: note = misc_feature - X can be any amino acid
  VARIANT 8..10: note = misc_feature - X can be any amino acid
XXNRXYYXXXE

SEQ ID NO: 229 - protein, 11 aa, synthetic construct
  REGION 1..11: note = epitope
  VARIANT 8: note = misc_feature - X can be any amino acid
LDNRLYYXVAE

SEQ ID NO: 230 - protein, 11 aa, synthetic construct
  REGION 1..11: note = epitope
LDNRLYYMVAE

SEQ ID NO: 231 - skipped

SEQ ID NO: 232 - protein, 8 aa, synthetic construct
  REGION 1..8: note = epitope
YIQAVLDR

SEQ ID NO: 233 - protein, 10 aa, synthetic construct
  REGION 1..10: note = epitope
  VARIANT 4: note = misc_feature - X can be any amino acid
  VARIANT 6..7: note = misc_feature - X can be any amino acid
  VARIANT 9: note = misc_feature - X can be any amino acid
GGYXQXXLXR

SEQ ID NO: 234 - protein, 10 aa, synthetic construct
  REGION 1..10: note = epitope
  VARIANT 6..7: note = misc_feature - X can be any amino acid
GGYIQXXLDR

SEQ ID NO: 235 - protein, 10 aa, synthetic construct
  REGION 1..10: note = epitope
GGYIQAVLDR

SEQ ID NO: 236 - protein, 8 aa, synthetic construct
  REGION 1..8: note = epitope
  VARIANT 2: note = misc_feature - X can be any amino acid
  VARIANT 4..6: note = misc_feature - X can be any amino acid
RXKXXXDY

SEQ ID NO: 237 - protein, 8 aa, synthetic construct
  REGION 1..8: note = epitope
RFKVFADY

SEQ ID NO: 238 - skipped

SEQ ID NO: 239 - protein, 6 aa, synthetic construct
  REGION 1..6: note = epitope
KKSNIR

SEQ ID NO: 240 - protein, 7 aa, synthetic construct
  REGION 1..7: note = epitope
  VARIANT 3..5: note = misc_feature - X can be any amino acid
KKXXXRN

SEQ ID NO: 241 - protein, 7 aa, synthetic construct
  REGION 1..7: note = epitope
  VARIANT 5: note = misc_feature - X can be any amino acid
KKSNXRN

SEQ ID NO: 242 - protein, 7 aa, synthetic construct
  REGION 1..7: note = epitope
KKSNIRN

SEQ ID NO: 243 - protein, 11 aa, synthetic construct
  REGION 1..11: note = epitope
  VARIANT 2..4: note = misc_feature - X can be any amino acid
  VARIANT 6..7: note = misc_feature - X can be any amino acid
  VARIANT 9..10: note = misc_feature - X can be any amino acid
LXXXLXXKXXI

SEQ ID NO: 244 - protein, 11 aa, synthetic construct
  REGION 1..11: note = epitope
  VARIANT 2: note = misc_feature - X can be any amino acid
  VARIANT 4: note = misc_feature - X can be any amino acid
  VARIANT 6..7: note = misc_feature - X can be any amino acid
  VARIANT 9..10: note = misc_feature - X can be any amino acid
LXDXLXXKXXI

SEQ ID NO: 245 - protein, 11 aa, synthetic construct
  REGION 1..11: note = epitope
LTDSLVSKAGI

SEQ ID NO: 246 - skipped

SEQ ID NO: 247 - protein, 11 aa, synthetic construct
  REGION 1..11: note = epitope
GKSTLTDSLVS

SEQ ID NO: 248 - skipped

SEQ ID NO: 249 - protein, 7 aa, synthetic construct
  REGION 1..7: note = epitope
  VARIANT 3..4: note = misc_feature - X can be any amino acid
EIXXAYL

SEQ ID NO: 250 - protein, 7 aa, synthetic construct
  REGION 1..7: note = epitope
EIAEAYL

SEQ ID NO: 251-253 - skipped (3 entries)

SEQ ID NO: 254 - protein, 7 aa, synthetic construct
  REGION 1..7: note = epitope
  VARIANT 4..5: note = misc_feature - X can be any amino acid
DDVXXDK

SEQ ID NO: 255 - protein, 7 aa, synthetic construct
  REGION 1..7: note = epitope
DDVQRDK

SEQ ID NO: 256 - protein, 7 aa, synthetic construct
  REGION 1..7: note = epitope
  VARIANT 2: note = misc_feature - X can be any amino acid
  VARIANT 4: note = misc_feature - X can be any amino acid
  VARIANT 6: note = misc_feature - X can be any amino acid
KXSXEXK

SEQ ID NO: 257 - protein, 7 aa, synthetic construct
  REGION 1..7: note = epitope
  VARIANT 4: note = misc_feature - X can be any amino acid
KLSXEDK

SEQ ID NO: 258 - protein, 7 aa, synthetic construct
  REGION 1..7: note = epitope
KLSDEDK

SEQ ID NO: 259 - protein, 7 aa, synthetic construct
  REGION 1..7: note = epiotope
  VARIANT 2..4: note = misc_feature - X can be any amino acid
QXXXDKC

SEQ ID NO: 260 - protein, 7 aa, synthetic construct
  REGION 1..7: note = epitope
  VARIANT 4: note = misc_feature - X can be any amino acid
QKIXDKC

SEQ ID NO: 261 - protein, 7 aa, synthetic construct
  REGION 1..7: note = epitope
QKILDKC

SEQ ID NO: 262 - skipped

SEQ ID NO: 263 - protein, 5 aa, synthetic construct
  REGION 1..5: note = epitope
DTCYY

SEQ ID NO: 264 - protein, 9 aa, synthetic construct
  REGION 1..9: note = epitope
  VARIANT 2: note = misc_feature - X can be any amino acid
  VARIANT 4: note = misc_feature - X can be any amino acid
  VARIANT 7..9: note = misc_feature - X can be any amino acid
VXVXKKXXX

SEQ ID NO: 265 - protein, 9 aa, synthetic construct
  REGION 1..9: note = epitope
  VARIANT 8: note = misc_feature - X can be any amino acid
VAVAKKGXE

SEQ ID NO: 266 - protein, 9 aa, synthetic construct
  REGION 1..9: note = epitope
VAVAKKGTE

SEQ ID NO: 267 - skipped

SEQ ID NO: 268 - protein, 8 aa, synthetic construct
  REGION 1..8: note = epitope
  VARIANT 1: note = misc_feature - X can be any amino acid
  VARIANT 3: note = misc_feature - X can be any amino acid
  VARIANT 5: note = misc_feature - X can be any amino acid
  VARIANT 8: note = misc_feature - X can be any amino acid
XKXGXGEX

SEQ ID NO: 269 - protein, 8 aa, synthetic construct
  REGION 1..8: note = epitope
LKDGAGEV

SEQ ID NO: 270 - skipped

SEQ ID NO: 271 - protein, 5 aa, synthetic construct
  REGION 1..5: note = epitope
YSKLM

SEQ ID NO: 272 - protein, 8 aa, synthetic construct
  REGION 1..8: note = epitope
  VARIANT 5..7: note = misc_feature - X can be any amino acid
VTNFXXXS

SEQ ID NO: 273 - protein, 8 aa, synthetic construct
  REGION 1..8: note = epitope
VTNFNLFS

SEQ ID NO: 274 - skipped

SEQ ID NO: 275 - protein, 9 aa, synthetic construct
  REGION 1..9: note = epitope
  VARIANT 2: note = misc_feature - X can be any amino acid
  VARIANT 5..6: note = misc_feature - X can be any amino acid
  VARIANT 8: note = misc_feature - X can be any amino acid
YXYNXXFXC

SEQ ID NO: 276 - protein, 9 aa, synthetic construct
  REGION 1..9: note = epitope
YGYNGAFRC

SEQ ID NO: 277 - skipped

SEQ ID NO: 278 - protein, 5 aa, synthetic construct
  REGION 1..5: note = epitope
  VARIANT 2: note = misc_feature - X can be any amino acid
FXCLV

SEQ ID NO: 279 - protein, 5 aa, synthetic construct
  REGION 1..5: note = epitope
FRCLV

SEQ ID NO: 280 - protein, 9 aa, synthetic construct
  REGION 1..9: note = epitope
  VARIANT 4..7: note = misc_feature - X can be any amino acid
YVKXXXXKI

SEQ ID NO: 281 - protein, 9 aa, synthetic construct
  REGION 1..9: note = epitope
  VARIANT 4: note = misc_feature - X can be any amino acid
  VARIANT 6: note = misc_feature - X can be any amino acid
YVKXVXEKI

SEQ ID NO: 282 - protein, 9 aa, synthetic construct
  REGION 1..9: note = epitope
YVKKVGEKI

SEQ ID NO: 283 - protein, 5 aa, synthetic construct
  REGION 1..5: note = epitope
  VARIANT 3: note = misc_feature - X can be any amino acid
NIXIP

SEQ ID NO: 284 - protein, 5 aa, synthetic construct
  REGION 1..5: note = epitope
NIVIP

SEQ ID NO: 285 - protein, 11 aa, synthetic construct
  REGION 1..11: note = epitope
  VARIANT 2: note = misc_feature - X can be any amino acid
  VARIANT 4..6: note = misc_feature - X can be any amino acid
  VARIANT 8..10: note = misc_feature - X can be any amino acid
SXEXXXKXXXF

SEQ ID NO: 286 - protein, 11 aa, synthetic construct
  REGION 1..11: note = epitope
  VARIANT 2: note = misc_feature - X can be any amino acid
  VARIANT 4: note = misc_feature - X can be any amino acid
  VARIANT 6: note = misc_feature - X can be any amino acid
  VARIANT 8..10: note = misc_feature - X can be any amino acid
SXEXCXKXXXF

SEQ ID NO: 287 - protein, 11 aa, synthetic construct
  REGION 1..11: note = epitope
SYEMCVKVDDF

SEQ ID NO: 288 - protein, 8 aa, synthetic construct
  REGION 1..8: note = epitope
  VARIANT 2: note = misc_feature - X can be any amino acid
  VARIANT 4: note = misc_feature - X can be any amino acid
  VARIANT 6..7: note = misc_feature - X can be any amino acid
KXDXFXXK

SEQ ID NO: 289 - protein, 8 aa, synthetic construct
  REGION 1..8: note = epitope
  VARIANT 2: note = misc_feature - X can be any amino acid
  VARIANT 4: note = misc_feature - X can be any amino acid
  VARIANT 6: note = misc_feature - X can be any amino acid
KXDXFXDK

SEQ ID NO: 290 - protein, 8 aa, synthetic construct
  REGION 1..8: note = epitope
KVDDFEDK

SEQ ID NO: 291 - skipped

SEQ ID NO: 292 - protein, 14 aa, synthetic construct
  REGION 1..14: note = epitope
EVIDYKPTPFVEHD

SEQ ID NO: 293 - skipped

SEQ ID NO: 294 - protein, 10 aa, synthetic construct
  REGION 1..10: note = epitope
  VARIANT 3..4: note = misc_feature - X can be any amino acid
  VARIANT 6..7: note = misc_feature - X can be any amino acid
  VARIANT 9: note = misc_feature - X can be any amino acid
DNXXYXXIXT

SEQ ID NO: 295 - protein, 10 aa, synthetic construct
  REGION 1..10: note = epitope
DNPKYRMIST

SEQ ID NO: 296 - skipped

SEQ ID NO: 297 - protein, 6 aa, synthetic construct
  REGION 1..6: note = epitope
YRMIST

SEQ ID NO: 298 - protein, 8 aa, synthetic construct
  REGION 1..8: note = epitope
  VARIANT 1..3: note = misc_feature - X can be any amino acid
  VARIANT 6: note = misc_feature - X can be any amino acid
XXXISXGG

SEQ ID NO: 299 - protein, 8 aa, synthetic construct
  REGION 1..8: note = epitope
  VARIANT 2: note = misc_feature - X can be any amino acid
  VARIANT 6: note = misc_feature - X can be any amino acid
YXMISXGG

SEQ ID NO: 300 - protein, 8 aa, synthetic construct
  REGION 1..8: note = epitope
YRMISTGG

SEQ ID NO: 301-302 - skipped (2 entries)

SEQ ID NO: 303 - protein, 6 aa, synthetic construct
  REGION 1..6: note = epitope
KSKVSD

SEQ ID NO: 304 - skipped

SEQ ID NO: 305 - protein, 9 aa, synthetic construct
  REGION 1..9: note = epitope
  VARIANT 5: note = misc_feature - X can be any amino acid
YKSKXSDLN

SEQ ID NO: 306 - protein, 9 aa, synthetic construct
  REGION 1..9: note = epitope
YKSKVSDLN

SEQ ID NO: 307 - skipped

SEQ ID NO: 308 - protein, 4 aa, synthetic construct
  REGION 1..4: note = epitope
VNKN

SEQ ID NO: 309 - protein, 7 aa, synthetic construct
  REGION 1..7: note = epitope
  VARIANT 2: note = misc_feature - X can be any amino acid
  VARIANT 5..6: note = misc_feature - X can be any amino acid
DXGRXXE

SEQ ID NO: 310 - protein, 7 aa, synthetic construct
  REGION 1..7: note = epitope
DVGRSAE

SEQ ID NO: 311 - skipped

SEQ ID NO: 312 - protein, 6 aa, synthetic construct
  REGION 1..6: note = epitope
KKAGDG

SEQ ID NO: 313 - skipped

SEQ ID NO: 314 - protein, 5 aa, synthetic construct
  REGION 1..5: note = epitope
SKKIK

SEQ ID NO: 315 - skipped

SEQ ID NO: 316 - protein, 11 aa, synthetic construct
  REGION 1..11: note = epitope
  VARIANT 2..5: note = misc_feature - X can be any amino acid
  VARIANT 7: note = misc_feature - X can be any amino acid
HXXXXQXKSSG

SEQ ID NO: 317 - protein, 11 aa, synthetic construct
  REGION 1..11: note = epitope
HVVEVQEKSSG

SEQ ID NO: 318 - skipped

SEQ ID NO: 319 - protein, 9 aa, synthetic construct
  REGION 1..9: note = epitope
  VARIANT 3: note = misc_feature - X can be any amino acid
  VARIANT 7: note = misc_feature - X can be any amino acid
IYXGKTXDI

SEQ ID NO: 320 - protein, 9 aa, synthetic construct
  REGION 1..9: note = epitope
IYFGKTKDI

SEQ ID NO: 321 - protein, 9 aa, synthetic construct
  REGION 1..9: note = epitiope
  VARIANT 1: note = misc_feature - X can be any amino acid
  VARIANT 3..4: note = misc_feature - X can be any amino acid
  VARIANT 6..7: note = misc_feature - X can be any amino acid
XDXXNXXRS

SEQ ID NO: 322 - protein, 9 aa, synthetic construct
  REGION 1..9: note = epitope
  VARIANT 3..4: note = misc_feature - X can be any amino acid
  VARIANT 6: note = misc_feature - X can be any amino acid
KDXXNXLRS

SEQ ID NO: 323 - protein, 9 aa, synthetic construct
  REGION 1..9: note = epitope
KDIVNGLRS

SEQ ID NO: 324 - protein, 12 aa, synthetic construct
  REGION 1..12: note = epitope
  VARIANT 6..7: note = misc_feature - X can be any amino acid
  VARIANT 9..12: note = misc_feature - X can be any amino acid
QKYRQXXKXXXX

SEQ ID NO: 325 - protein, 12 aa, synthetic construct
  REGION 1..12: note = epitope
  VARIANT 6..7: note = misc_feature - X can be any amino acid
  VARIANT 9: note = misc_feature - X can be any amino acid
  VARIANT 11: note = misc_feature - X can be any amino acid
QKYRQXXKXLXQ

SEQ ID NO: 326 - protein, 12 aa, synthetic construct
  REGION 1..12: note = epitope
QKYRQLQKELSQ

SEQ ID NO: 327 - protein, 11 aa, synthetic construct
  REGION 1..11: note = epitope
  VARIANT 2..5: note = misc_feature - X can be any amino acid
  VARIANT 7..8: note = misc_feature - X can be any amino acid
TXXXXDXXEGK

SEQ ID NO: 328 - protein, 11 aa, synthetic construct
  REGION 1..11: note = epitope
  VARIANT 2..4: note = misc_feature - X can be any amino acid
TXXXLDFREGK

SEQ ID NO: 329 - protein, 11 aa, synthetic construct
  REGION 1..11: note = epitope
TVTALDFREGK

SEQ ID NO: 330 - skipped

SEQ ID NO: 331 - protein, 9 aa, synthetic construct
  REGION 1..9: note = epitope
  VARIANT 4..5: note = misc_feature - X can be any amino acid
  VARIANT 7..8: note = misc_feature - X can be any amino acid
FREXXEXXD

SEQ ID NO: 332 - protein, 9 aa, synthetic construct
  REGION 1..9: note = epitope
FREGKEFKD

SEQ ID NO: 333 - protein, 9 aa, synthetic construct
  REGION 1..9: note = epitope
  VARIANT 2..3: note = misc_feature - X can be any amino acid
  VARIANT 7..8: note = misc_feature - X can be any amino acid
LXXNFTXXK

SEQ ID NO: 334 - protein, 9 aa, synthetic construct
  REGION 1..9: note = epitope
  VARIANT 2: note = misc_feature - X can be any amino acid
LXINFTNEK

SEQ ID NO: 335 - protein, 9 aa, synthetic construct
  REGION 1..9: note = epitope
LCINFTNEK

SEQ ID NO: 336 - protein, 10 aa, synthetic construct
  REGION 1..10: note = epitope
  VARIANT 1: note = misc_feature - X can be any amino acid
  VARIANT 3: note = misc_feature - X can be any amino acid
  VARIANT 7..9: note = misc_feature - X can be any amino acid
XYXPPPXXXK

SEQ ID NO: 337 - protein, 10 aa, synthetic construct
  REGION 1..10: note = epitope
  VARIANT 3: note = misc_feature - X can be any amino acid
  VARIANT 7..9: note = misc_feature - X can be any amino acid
LYXPPPXXXK

SEQ ID NO: 338 - protein, 10 aa, synthetic construct
  REGION 1..10: note = epitope
LYPPPPPEDK

SEQ ID NO: 339 - protein, 8 aa, synthetic construct
  REGION 1..8: note = epitope
  VARIANT 1..2: note = misc_feature - X can be any amino acid
  VARIANT 5: note = misc_feature - X can be any amino acid
  VARIANT 7: note = misc_feature - X can be any amino acid
XXDEXVXK

SEQ ID NO: 340 - protein, 8 aa, synthetic construct
  REGION 1..8: note = epitope
  VARIANT 2: note = misc_feature - X can be any amino acid
  VARIANT 5: note = misc_feature - X can be any amino acid
  VARIANT 7: note = misc_feature - X can be any amino acid
SXDEXVXK

SEQ ID NO: 341 - protein, 8 aa, synthetic construct
  REGION 1..8: note = epitope
SMDESVAK

SEQ ID NO: 342 - protein, 10 aa, synthetic construct
  REGION 1..10: note = epitope
  VARIANT 1..2: note = misc_feature - X can be any amino acid
  VARIANT 4..6: note = misc_feature - X can be any amino acid
  VARIANT 9: note = misc_feature - X can be any amino acid
XXDXXXAKXT

SEQ ID NO: 343 - protein, 10 aa, synthetic construct
  REGION 1..10: note = epitope
  VARIANT 2: note = misc_feature - X can be any amino acid
  VARIANT 5: note = misc_feature - X can be any amino acid
  VARIANT 9: note = misc_feature - X can be any amino acid
SXDEXVAKXT

SEQ ID NO: 344 - protein, 10 aa, synthetic construct
  REGION 1..10: note = epitope
SMDESVAKLT

SEQ ID NO: 345 - protein, 9 aa, synthetic construct
  REGION 1..9: note = epitope
  VARIANT 2..5: note = misc_feature - X can be any amino acid
  VARIANT 7: note = misc_feature - X can be any amino acid
VXXXXKXKK

SEQ ID NO: 346 - protein, 9 aa, synthetic construct
  REGION 1..9: note = epitope
  VARIANT 3..5: note = misc_feature - X can be any amino acid
  VARIANT 7: note = misc_feature - X can be any amino acid
VAXXXKXKK

SEQ ID NO: 347 - protein, 9 aa, synthetic construct
  REGION 1..9: note = epitope
VAKLTKEKK

SEQ ID NO: 348 - skipped

SEQ ID NO: 349 - protein, 6 aa, synthetic construct
  REGION 1..6: note = epitope
  VARIANT 2..3: note = misc_feature - X can be any amino acid
KXXKEK

SEQ ID NO: 350 - protein, 6 aa, synthetic construct
  REGION 1..6: note = epitope
KLTKEK

SEQ ID NO: 351 - protein, 10 aa, synthetic construct
  REGION 1..10: note = epitope
  VARIANT 1: note = misc_feature - X can be any amino acid
  VARIANT 3..5: note = misc_feature - X can be any amino acid
  VARIANT 7: note = misc_feature - X can be any amino acid
  VARIANT 9: note = misc_feature - X can be any amino acid
XNXXXKXKXK

SEQ ID NO: 352 - protein, 10 aa, synthetic construct
  REGION 1..10: note = epitope
  VARIANT 1: note = misc_feature - X can be any amino acid
  VARIANT 3..5: note = misc_feature - X can be any amino acid
  VARIANT 7: note = misc_feature - X can be any amino acid
XNXXXKXKTK

SEQ ID NO: 353 - protein, 10 aa, synthetic construct
  REGION 1..10: note = epitope
VNTLTKAKTK

SEQ ID NO: 354 - skipped

SEQ ID NO: 355 - protein, 8 aa, synthetic construct
  REGION 1..8: note = epitope
  VARIANT 2..4: note = misc_feature - X can be any amino acid
  VARIANT 6: note = misc_feature - X can be any amino acid
DXXXSXRK

SEQ ID NO: 356 - protein, 8 aa, synthetic construct
  REGION 1..8: note = epitope
DLERSKRK

SEQ ID NO: 357 - skipped

SEQ ID NO: 358 - protein, 7 aa, synthetic construct
  REGION 1..7: note = epitope
  VARIANT 2: note = misc_feature - X can be any amino acid
  VARIANT 5: note = misc_feature - X can be any amino acid
SXRKXEG

SEQ ID NO: 359 - protein, 7 aa, synthetic construct
  REGION 1..7: note = epitope
SKRKLEG

SEQ ID NO: 360 - skipped

SEQ ID NO: 361 - protein, 8 aa, synthetic construct
  REGION 1..8: note = epitope
  VARIANT 2: note = misc_feature - X can be any amino acid
  VARIANT 4..5: note = misc_feature - X can be any amino acid
EXRXXEEK

SEQ ID NO: 362 - protein, 8 aa, synthetic construct
  REGION 1..8: note = epitope
EARNAEEK

SEQ ID NO: 363 - protein, 9 aa, synthetic construct
  REGION 1..9: note = epitope
  VARIANT 1..3: note = misc_feature - X can be any amino acid
  VARIANT 5: note = misc_feature - X can be any amino acid
  VARIANT 7: note = misc_feature - X can be any amino acid
XXXEXKXKK

SEQ ID NO: 364 - protein, 9 aa, synthetic construct
  REGION 1..9: note = epitope
  VARIANT 2..3: note = misc_feature - X can be any amino acid
  VARIANT 7: note = misc_feature - X can be any amino acid
RXXEEKXKK

SEQ ID NO: 365 - protein, 9 aa, synthetic construct
  REGION 1..9: note = epitope
RNAEEKAKK

SEQ ID NO: 366 - skipped

SEQ ID NO: 367 - protein, 10 aa, synthetic construct
  REGION 1..10: note = epitope
GGYIQAVLDR

SEQ ID NO: 368 - skipped

SEQ ID NO: 369 - protein, 5 aa, synthetic construct
  REGION 1..5: note = epitope
  VARIANT 3: note = misc_feature - X can be any amino acid
NLXEN

SEQ ID NO: 370 - protein, 5 aa, synthetic construct
  REGION 1..5: note = epitope
NLCEN

SEQ ID NO: 371 - protein, 10 aa, synthetic construct
  REGION 1..10: note = epitope
  VARIANT 2: note = misc_feature - X can be any amino acid
  VARIANT 4..5: note = misc_feature - X can be any amino acid
  VARIANT 9..10: note = misc_feature - X can be any amino acid
KXTXXKKKXX

SEQ ID NO: 372 - protein, 10 aa, synthetic construct
  REGION 1..10: note = epitope
  VARIANT 2: note = misc_feature - X can be any amino acid
  VARIANT 4..5: note = misc_feature - X can be any amino acid
KXTXXKKKPV

SEQ ID NO: 373 - protein, 10 aa, synthetic construct
  REGION 1..10: note = epitope
KATKKKKKPV

SEQ ID NO: 374 - protein, 8 aa, synthetic construct
  REGION 1..8: note = epitope
  VARIANT 2: note = misc_feature - X can be any amino acid
  VARIANT 7..8: note = misc_feature - X can be any amino acid
KXKPVVXX

SEQ ID NO: 375 - protein, 8 aa, synthetic construct
  REGION 1..8: note = epitope
  VARIANT 2: note = misc_feature - X can be any amino acid
KXKPVVDE

SEQ ID NO: 376 - protein, 8 aa, synthetic construct
  REGION 1..8: note = epitope
KKKPVVDE

SEQ ID NO: 377 - skipped

SEQ ID NO: 378 - protein, 10 aa, synthetic construct
  REGION 1..10: note = epitope
  VARIANT 2..3: note = misc_feature - X can be any amino acid
  VARIANT 5: note = misc_feature - X can be any amino acid
  VARIANT 9: note = misc_feature - X can be any amino acid
YXXIXFEYXI

SEQ ID NO: 379 - protein, 10 aa, synthetic construct
  REGION 1..10: note = epitope
YEKIAFEYGI

SEQ ID NO: 380 - protein, 8 aa, synthetic construct
  REGION 1..8: note = epitope
  VARIANT 1..2: note = misc_feature - X can be any amino acid
  VARIANT 4: note = misc_feature - X can be any amino acid
  VARIANT 6: note = misc_feature - X can be any amino acid
XXIXDXRG

SEQ ID NO: 381 - protein, 8 aa, synthetic construct
  REGION 1..8: note = epitope
  VARIANT 2: note = misc_feature - X can be any amino acid
  VARIANT 4: note = misc_feature - X can be any amino acid
  VARIANT 6: note = misc_feature - X can be any amino acid
YXIXDXRG

SEQ ID NO: 382 - protein, 8 aa, synthetic construct
  REGION 1..8: note = epitope
YGITDLRG

SEQ ID NO: 383 - protein, 7 aa, synthetic construct
  REGION 1..7: note = epitope
  VARIANT 2: note = misc_feature - X can be any amino acid
  VARIANT 5..6: note = misc_feature - X can be any amino acid
DXRGXXK

SEQ ID NO: 384 - protein, 7 aa, synthetic construct
  REGION 1..7: note = epitope
DLRGLLK

SEQ ID NO: 385 - protein, 8 aa, synthetic construct
  REGION 1..8: note = epitope
  VARIANT 3..6: note = misc_feature - X can be any amino acid
KMXXXXPK

SEQ ID NO: 386 - protein, 8 aa, synthetic construct
  REGION 1..8: note = epitope
KMKTVEPK

SEQ ID NO: 387 - protein, 10 aa, synthetic construct
  REGION 1..10: note = epitope
  VARIANT 2..3: note = misc_feature - X can be any amino acid
  VARIANT 5..7: note = misc_feature - X can be any amino acid
KXXKXXXPKH

SEQ ID NO: 388 - protein, 10 aa, synthetic construct
  REGION 1..10: note = epitope
KKMKTVEPKH

SEQ ID NO: 389 - skipped

SEQ ID NO: 390 - protein, 7 aa, synthetic construct
  REGION 1..7: note = epitope
KHSDAFL

SEQ ID NO: 391 - protein, 6 aa, synthetic construct
  REGION 1..6: note = epitope
  VARIANT 5..6: note = misc_feature - X can be any amino acid
KGKKXX

SEQ ID NO: 392 - protein, 6 aa, synthetic construct
  REGION 1..6: note = epitope
KGKKIV

SEQ ID NO: 393 - protein, 10 aa, synthetic construct
  REGION 1..10: note = epitope
  VARIANT 2: note = misc_feature - X can be any amino acid
  VARIANT 4..6: note = misc_feature - X can be any amino acid
  VARIANT 8..9: note = misc_feature - X can be any amino acid
KXKXXXLXXE

SEQ ID NO: 394 - protein, 10 aa, synthetic construct
  REGION 1..10: note = epitope
  VARIANT 2: note = misc_feature - X can be any amino acid
  VARIANT 4..6: note = misc_feature - X can be any amino acid
  VARIANT 9: note = misc_feature - X can be any amino acid
KXKXXXLQXE

SEQ ID NO: 395 - protein, 10 aa, synthetic construct
  REGION 1..10: note = epitope
KGKKIVLQVE

SEQ ID NO: 396 - skipped

SEQ ID NO: 397 - protein, 4 aa, synthetic construct
  REGION 1..4: note = epitope
THKY

SEQ ID NO: 398 - protein, 9 aa, synthetic construct
  REGION 1..9: note = epitope
  VARIANT 3..4: note = misc_feature - X can be any amino acid
  VARIANT 7..9: note = misc_feature - X can be any amino acid
KYXXKKXXX

SEQ ID NO: 399 - protein, 9 aa, synthetic construct
  REGION 1..9: note = epitope
  VARIANT 3..4: note = misc_feature - X can be any amino acid
KYXXKKDGL

SEQ ID NO: 400 - protein, 9 aa, synthetic construct
  REGION 1..9: note = epitope
KYRFKKDGL

SEQ ID NO: 401 - protein, 7 aa, synthetic construct
  REGION 1..7: note = epitope
  VARIANT 2..4: note = misc_feature - X can be any amino acid
YXXXPDG

SEQ ID NO: 402 - protein, 7 aa, synthetic construct
  REGION 1..7: note = epitope
YTFIPDG

SEQ ID NO: 403 - protein, 10 aa, synthetic construct
  REGION 1..10: note = epitope
  VARIANT 4..5: note = misc_feature - X can be any amino acid
  VARIANT 7: note = misc_feature - X can be any amino acid
  VARIANT 9: note = misc_feature - X can be any amino acid
DGYXXSXSXK

SEQ ID NO: 404 - protein, 10 aa, synthetic construct
  REGION 1..10: note = epitope
  VARIANT 4: note = misc_feature - X can be any amino acid
  VARIANT 9: note = misc_feature - X can be any amino acid
DGYXLSLSXK

SEQ ID NO: 405 - protein, 10 aa, synthetic construct
  REGION 1..10: note = epitope
DGYALSLSAK

SEQ ID NO: 406 - skipped

SEQ ID NO: 407 - protein, 5 aa, synthetic construct
  REGION 1..5: note = epitope
PVTGY

SEQ ID NO: 408 - protein, 12 aa, synthetic construct
  REGION 1..12: note = epitope
  VARIANT 2..8: note = misc_feature - X can be any amino acid
  VARIANT 11: note = misc_feature - X can be any amino acid
QXXXXXXXDKXN

SEQ ID NO: 409 - protein, 12 aa, synthetic construct
  REGION 1..12: note = epitope
QKYIRRVGDKIN

SEQ ID NO: 410 - protein, 10 aa, synthetic construct
  REGION 1..10: note = epitope
  VARIANT 2..6: note = misc_feature - X can be any amino acid
  VARIANT 8: note = misc_feature - X can be any amino acid
YXXXXXDXKT

SEQ ID NO: 411 - protein, 10 aa, synthetic construct
  REGION 1..10: note = epitope
YTIQKADKKT

SEQ ID NO: 412 - skipped

SEQ ID NO: 413 - protein, 8 aa, synthetic construct
  REGION 1..8: note = epitope
YTIQKADK

SEQ ID NO: 414 - skipped

SEQ ID NO: 415 - protein, 4 aa, synthetic construct
  REGION 1..4: note = epitope
GGYS

SEQ ID NO: 416 - skipped

SEQ ID NO: 417 - protein, 6 aa, synthetic construct
  REGION 1..6: note = epitope
YSVFAG

SEQ ID NO: 418 - protein, 9 aa, synthetic construct
  REGION 1..9: note = epitope
  VARIANT 1: note = misc_feature - X can be any amino acid
  VARIANT 6: note = misc_feature - X can be any amino acid
  VARIANT 9: note = misc_feature - X can be any amino acid
XPVGSXSKX

SEQ ID NO: 419 - protein, 9 aa, synthetic construct
  REGION 1..9: note = epitope
  VARIANT 6: note = misc_feature - X can be any amino acid
EPVGSXSKV

SEQ ID NO: 420 - protein, 9 aa, synthetic construct
  REGION 1..9: note = epitope
EPVGSRSKV

SEQ ID NO: 421 - skipped

SEQ ID NO: 422 - protein, 12 aa, synthetic construct
  REGION 1..12: note = epitope
  VARIANT 3: note = misc_feature - X can be any amino acid
  VARIANT 5: note = misc_feature - X can be any amino acid
  VARIANT 7..9: note = misc_feature - X can be any amino acid
  VARIANT 11: note = misc_feature - X can be any amino acid
SYXRXLXXXAXK

SEQ ID NO: 423 - protein, 12 aa, synthetic construct
  REGION 1..12: note = epitope
SYGRALQASALK

SEQ ID NO: 424 - skipped

SEQ ID NO: 425 - protein, 7 aa, synthetic construct
  REGION 1..7: note = epitope
SALKAWG

SEQ ID NO: 426 - protein, 8 aa, synthetic construct
  REGION 1..8: note = epitope
  VARIANT 6..8: note = misc_feature - X can be any amino acid
RYLGKXXX

SEQ ID NO: 427 - protein, 8 aa, synthetic construct
  REGION 1..8: note = epitope
  VARIANT 7: note = misc_feature - X can be any amino acid
RYLGKGXV

SEQ ID NO: 428 - protein, 8 aa, synthetic construct
  REGION 1..8: note = epitope
RYLGKGTV

SEQ ID NO: 429 - protein, 10 aa, synthetic construct
  REGION 1..10: note = epitope
  VARIANT 2..3: note = misc_feature - X can be any amino acid
  VARIANT 5: note = misc_feature - X can be any amino acid
  VARIANT 7..9: note = misc_feature - X can be any amino acid
YXXIXDXXXH

SEQ ID NO: 430 - protein, 10 aa, synthetic construct
  REGION 1..10: note = epitope
  VARIANT 2..3: note = misc_feature - X can be any amino acid
  VARIANT 5: note = misc_feature - X can be any amino acid
  VARIANT 8: note = misc_feature - X can be any amino acid
YXXIXDLXGH

SEQ ID NO: 431 - protein, 10 aa, synthetic construct
  REGION 1..10: note = epitope
YRHIADLAGH

SEQ ID NO: 432 - protein, 10 aa, synthetic construct
  REGION 1..10: note = epitope
  VARIANT 2..4: note = misc_feature - X can be any amino acid
  VARIANT 6: note = misc_feature - X can be any amino acid
  VARIANT 9: note = misc_feature - X can be any amino acid
DXXXHXDVXL

SEQ ID NO: 433 - protein, 10 aa, synthetic construct
  REGION 1..10: note = epitope
  VARIANT 2..4: note = misc_feature - X can be any amino acid
DXXXHKDVIL

SEQ ID NO: 434 - protein, 10 aa, synthetic construct
  REGION 1..10: note = epitope
DLAGHKDVIL

SEQ ID NO: 435 - protein, 10 aa, synthetic construct
  REGION 1..10: note = epitope
  VARIANT 3..6: note = misc_feature - X can be any amino acid
IKXXXXKDAT

SEQ ID NO: 436 - protein, 10 aa, synthetic construct
  REGION 1..10: note = epitope
IKAKYGKDAT

SEQ ID NO: 437 - protein, 15 aa, synthetic construct
  REGION 1..15: note = epitope
  VARIANT 2..8: note = misc_feature - X can be any amino acid
  VARIANT 10..12: note = misc_feature - X can be any amino acid
YXXXXXXXDXXXGRF

SEQ ID NO: 438 - protein, 15 aa, synthetic construct
  REGION 1..15: note = epitope
  VARIANT 2..8: note = misc_feature - X can be any amino acid
  VARIANT 12: note = misc_feature - X can be any amino acid
YXXXXXXXDSTXGRF

SEQ ID NO: 439 - protein, 15 aa, synthetic construct
  REGION 1..15: note = epitope
YMVYMFKYDSTHGRF

SEQ ID NO: 440 - protein, 8 aa, synthetic construct
  REGION 1..8: note = epitope
  VARIANT 3..4: note = misc_feature - X can be any amino acid
  VARIANT 6..7: note = misc_feature - X can be any amino acid
SYXXIXXV

SEQ ID NO: 441 - protein, 8 aa, synthetic construct
  REGION 1..8: note = epitope
SYDAIKKV

SEQ ID NO: 442 - protein, 6 aa, synthetic construct
  REGION 1..6: note = epitope
  VARIANT 4..5: note = misc_feature - X can be any amino acid
IKKXXK

SEQ ID NO: 443 - protein, 6 aa, synthetic construct
  REGION 1..6: note = epitope
  VARIANT 5: note = misc_feature - X can be any amino acid
IKKVXK

SEQ ID NO: 444 - protein, 6 aa, synthetic construct
  REGION 1..6: note = epitope
IKKVVK

SEQ ID NO: 445 - protein, 363 aa, synthetic construct
  REGION 1..363: note = aldolase a, fructose-bisphosphate 2
MPHAFPFLTPDQKKELSDIAHKIVAQGKGILAADESTGSVAKRFQSINTENTEENRRLYRQLLFTADERAGPCIGGVIFFHETLYQKTDAGKTFPQHVKSRGWLVGIKVDKGVVPLAGTNGETTTQGLDGLYERCAQYKKDGCDFAKWRCVLKITSTTPSRLAIMENCNVLARYASICQMHGIVPIVEPEILPDGDHDLKRTQYVTEKVLAAMYKALSDHHVYLEGTLLKPNMVTAGHSCSHKYTHQDIAMATITALRRTVPPAVPGITFLSGGQSEEEASINLNVMNQCPLHRPWAITFSYGRALQASALKAWGGKPKNGKAAQEEFIKRALANSLACQGKYVSSGDSAAAGDSLFVANHAY

SEQ ID NO: 446 - protein, 434 aa, synthetic construct
  REGION 1..434: note = beta-enolase
MSITKIHAREILDSRGNPTVEVDLYTAKGRFRAAVPSGASTGVHEALELRDGDKSRYLGKGTVKAVDHVNKDIAAKLIEKKFSVVDQEKIDHFMLELDGTENKSKFGANAILGVSLAVCKAGAAEKGVPLYRHIADLAGHKDVILPCPAFNVINGGSHAGNKLAMQEFMILPIGASNFHEAMRIGAEVYHNLKNVIKAKYGKDATNVGDEGGFAPNILENNEALELLKTAIEKAGYPDKIIIGMDVAASEFYKAGKYDLDFKSPDDPARYITGDQLGDLYKSFIKGYPVQSIEDPFDQDDWAAWTKFTAAVDIQVVGDDLTVTNPKRIQQAVEKKACNCLLLKVNQIGSVTESIKACKLAQSNGWGVMVSHRSGETEDTFIADLVVGLCTGQIKTGAPCRSERLAKYNQLMRIEEELGAKAKFAGKDYRHPKIN

SEQ ID NO: 447 - protein, 334 aa, synthetic construct
  REGION 1..334: note = glyceraldehyde-3-phosphate dehydrogenase
MVKVGVNGFGRIGRLVTRAAFHSKKGVEIVAINDPFIDLDYMVYMFKYDSTHGRFHGEVKAEGGKLVIDGHKITVFHERDPANIKWGDAGATYVVESTGVFTTIEKASTHLEGGAKRVVISAPSADAPMFVMGVNHEKYDNSLKVVSNASCTTNCLAPLAKVIHDNYHIIEGLMSTVHGVTATQKTVDGPSGKLWRDGRGASQNIIPASTGAAKAVGKVIPELNGKITGMAFRVPTPNVSVVDLTVRLEKPASYDAIKKVVKAAADGPMKGILGYTEQQVVSSDFNGDTHSSIFDAGAGIALNDHFVKLVTWYDNEFGYSNRVIDLMAHMATKE

SEQ ID NO: 448 - DNA, 1092 bp, synthetic construct
  misc_feature 1..1092: note = aldolase a, fructose-bisphosphate 2
  source 1..1092: mol_type = other DNA
atgcctcacgcattccccttcctcactcctgaccagaagaaggagcttagtgacattgctcacaagatcgtcgcccagggcaagggaatcctcgccgctgatgagtctaccggcagcgttgccaagcgcttccagagcatcaacactgagaacacagaggagaacaggagactgtaccgtcagctgctcttcaccgctgacgagcgtgccggtccttgcattggtggcgtcatcttcttccacgagaccctgtaccagaaaaccgatgccggcaagaccttcccccagcacgtcaagtccagaggctggcttgtaggcatcaaggttgacaaaggtgtcgtccccctcgccggaaccaacggagagaccaccacccagggtttggatggcctgtatgagcgttgcgcccagtataagaaggacggttgcgacttcgccaagtggcgttgtgtgctgaaaatcacctccaccaccccctctcgccttgctatcatggagaattgcaatgtcctggcccgttatgccagcatctgccagatgcacggcattgtccccattgttgagcccgagatcctccccgacggtgaccatgacctgaagcgcacccagtacgtgactgagaaggtcctggccgcaatgtacaaggctctgtccgaccaccacgtctacctggagggtaccctcctgaagcccaacatggtcactgctggacattcctgctcacacaagtacacccaccaggacatcgccatggccactattaccgccctgcgccgtaccgtgcccccagcagtccctggcatcaccttcctgtccggtggccagtctgaggaggaagcctccatcaacctgaatgtcatgaaccagtgccccctgcacaggccatgggccattactttctcctatggccgtgccctccaggcctccgcccttaaggcatggggtggcaagcccaagaacggcaaggctgcccaggaggagttcatcaagagagctctggccaacagcctggcctgccaaggcaagtatgtttcttccggagacagcgccgctgctggagattcactgttcgtggccaaccatgcttattaa

SEQ ID NO: 449 - DNA, 1305 bp, synthetic construct
  misc_feature 1..1305: note = beta-enolase
  source 1..1305: mol_type = other DNA
atgtctatcactaagatccatgcccgagagatcctcgactccagaggaaaccccaccgtggaggtggacctctacaccgccaaaggccgtttcagagcagccgtgcccagcggtgcttctaccggtgtccatgaggctctggagctgagagatggagacaagtcacgctacctgggaaaaggtaccgttaaggctgtggatcacgtaaacaaggacatcgctgccaaactgatcgagaagaagttcagcgttgttgaccaggagaagatcgaccatttcatgctggagttggacggaactgagaacaagtctaagtttggagccaacgccatcctgggcgtttctctggcagtctgcaaggcaggagcagcagagaagggagtgcccctgtaccgtcacatcgctgacctggccggacacaaggacgtcatcctgccctgccccgccttcaatgtgatcaacggtggtagccatgctgggaacaagctggccatgcaggagttcatgatcctgcccatcggagcgtctaacttccacgaggccatgaggatcggagctgaggtttaccacaacctgaagaacgtgatcaaggccaagtacggaaaggatgccaccaacgtaggcgatgagggcggcttcgcccccaacatcctggagaacaacgaggctctggagctcctgaagacagccattgagaaggccggctacccagacaagatcatcatcggcatggacgtggctgcctctgagttctacaaggcaggaaagtacgacctggacttcaagtcacctgacgaccctgccaggtacatcaccggggatcagctgggagatctgtacaagagcttcatcaagggatatcccgtccagtccattgaggatcccttcgatcaggatgattgggctgcatggacaaagttcaccgccgccgtcgacatccaggtggtgggtgatgatctgaccgtgaccaaccccaagcgtatccagcaggccgtggagaagaaggcctgtaactgcctgctcctcaaggtcaaccagattggctccgtcacagagtccatcaaggcgtgtaaactggcccagtctaacggatggggtgtgatggtgtctcatcgttccggagagacagaggataccttcatcgctgacctggtggtcggactctgcactggacagatcaagactggtgccccctgtagatcagagcgtctggccaaatacaaccagctgatgaggattgaagaggagcttggagccaaggccaagttcgcaggcaaggactaccgtcaccccaaaatcaactaa

SEQ ID NO: 450 - DNA, 1005 bp, synthetic construct
  misc_feature 1..1005: note = glyceraldehyde-3-phosphate dehydrogenase
  source 1..1005: mol_type = other DNA
atggtgaaagtaggtgtcaatggattcggccgtatcgggcgtctggtgacccgtgctgcattccactccaagaagggagttgagattgtggccatcaacgaccccttcatcgacctggactacatggtctacatgttcaagtatgactccacccacggacgtttccatggtgaggtcaaggctgagggtggcaagctggtcatcgatggacacaaaatcactgtgttccacgagagagacccagctaacatcaagtggggagatgctggtgccacctatgtagttgagtcaacaggtgtcttcaccaccattgagaaggcctctacccatcttgaaggtggtgccaagagggttgtcatctccgctcccagcgctgatgcacccatgttcgtcatgggcgtcaaccacgagaagtacgataactccctcaaggttgtcagcaatgcttcatgcacaaccaactgcctggctcccctggccaaggtcatccacgataactaccacatcattgagggtctgatgagcacagttcacggcgtcaccgccacccagaagactgttgatggtccttctggaaagctgtggagggatggacgtggcgccagccagaacatcatccctgcctccaccggagcagcaaaggctgtcggcaaggtcatccccgagctgaacggcaagatcactggcatggccttccgtgtccccacccccaacgtctcagtggtggacctgaccgtccgtctggagaagcctgccagctacgacgccatcaagaaggttgtcaaggctgccgctgatggacccatgaagggaattcttggatacacggagcaacaggttgtgtcttcagacttcaacggcgacacccactcctccatctttgatgccggcgctggcattgctctgaacgaccacttcgtcaagctggttacatggtatgacaacgagttcggctacagcaaccgcgtcattgacctgatggctcacatggccaccaaggagtaa

SEQ ID NO: 451 - protein, 15 aa, synthetic construct
  REGION 1..15: note = epitope
AMVLVKMKETAEAYL